(19) United States
(12) Patent Application Publication
(10) Pub. No.: US 2019/0047951 A1
(43) Pub. Date: Feb. 14, 2019

(54) AZETIDINE MODULATORS OF THE SPHINGOSINE 1-PHOSPHATE RECEPTOR

(71) Applicant: AUSPEX PHARMACEUTICALS, INC., La Jolla, CA (US)

(72) Inventors: Chengzhi ZHANG, La Jolla, CA (US); Justin CHAKMA, La Jolla, CA (US)

(21) Appl. No.: 16/066,421

(22) PCT Filed: Jan. 3, 2017
(86) PCT No.: PCT/US2017/012009

§ 371 Date: Jun. 27, 2018

Related U.S. Application Data
(60) Provisional application No. 62/274,558, filed on Jan. 4, 2016.

(51) Int. Cl. C07D 205/04; A61P 35/00; C07B 59/00

(52) U.S. Cl. CPC C07D 205/04; C07B 2200/05; C07B 59/002; A61P 35/00

(57) ABSTRACT

Described are deuterium-substituted azetidine compounds of Formula (I), which are modulators of sphingosine 1-phosphate receptor. Also described are pharmaceutical compositions comprising the deuterium-substituted azetidine compounds, and methods of use thereof.

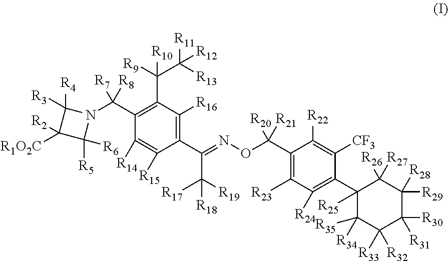

### CROSS-REFERENCE TO RELATED APPLICATIONS

This application claims the benefit of U.S. Provisional Application No. 62/274,558, filed Jan. 4, 2016, the disclosure of which is hereby incorporated by reference in its entirety.

### TECHNICAL FIELD

Disclosed herein are new azetidine compounds and compositions and their application as pharmaceuticals for the treatment of disorders. Methods of modulation of sphingosine 1-phosphate receptor activity in a subject are also provided for the treatment of disorders such as multiple sclerosis, secondary progressive multiple sclerosis, acute or chronic rejection of cell, tissue or organ allo- or xenografts, delayed graft function, graft versus host disease, autoimmune diseases, rheumatoid arthritis, systemic lupus erythematosus, Hashimoto's thyroiditis, myasthenia gravis, diabetes type I or II and the disorders associated therewith, vasculitis, pernicious anemia, Sjogren's syndrome, uveitis, psoriasis, Graves ophthalmopathy, alopecia areata, allergic diseases, allergic asthma, atopic dermatitis, allergic rhinitis and/or conjunctivitis, allergic contact dermatitis, inflammatory diseases, inflammatory bowel disease, Crohn's disease, ulcerative colitis, intrinsic asthma, inflammatory lung injury, inflammatory liver injury, inflammatory glomerular injury, atherosclerosis, osteoarthritis, irritant contact dermatitis, eczematous dermatitises, seborrheic dermatitis, cutaneous manifestations of immunologically-mediated disorders, inflammatory eye disease, keratoconjunctivitis, inflammatory myopathy; myocarditis, hepatitis, ischemia/reperfusion injury, myocardial infarction, stroke, gut ischemia, renal failure, hemorrhage shock, traumatic shock, T cell lymphoma, T cell leukemia, infectious diseases, toxic shock, septic shock, adult respiratory distress syndrome, viral infections, AIDS, viral hepatitis, chronic bacterial infection, muscle diseases, polymyositis, senile dementia, pancreatic islet transplant, stem cell transplant, bone marrow transplant, corneal tissue transplant, neuronal tissue transplant, heart transplant, lung transplant, combined heart-lung transplant, kidney transplant, liver transplant, bowel transplant, pancreas transplant, trachea transplant, esophagus transplant, cancer chemotherapy, cancer chemotherapy of solid tumors, breast cancer, peripheral neuropathy, acute demyelinating neuropathies, chronic demyelinating neuropathies, Guillain-Barre syndrome, chronic inflammatory demyelinating polyradiculoneuropathy, multifocal motor neuropathy with conduction block, paraproteinaemic demyelinating peripheral neuropathy, acute inflammatory demyelinating polyneuropathy, polymyositis, dermatomyositis, nerve-muscle diseases, muscular dystrophy, and inclusion body myositis.

### BACKGROUND

Siponimod (BAF312; CAS #1230487; 1-(4-[1-[(E)-4-cyclohexyl-3-trifluoromethyl-benzyloxyimino]-ethyl]-2-ethyl-benzyl)-azetidine-3-carboxylic acid) is a sphingosine 1-phosphate receptor (S1PR) agonist. Siponimod is currently under investigation for the treatment of multiple sclerosis. Siponimod has also shown promise in treating secondary progressive multiple sclerosis, acute or chronic rejection of cell, tissue or organ allo- or xenografts, delayed graft function, graft versus host disease, autoimmune diseases, rheumatoid arthritis, systemic lupus erythematosus, Hashimoto's thyroiditis, myasthenia gravis, diabetes type I or II and the disorders associated therewith, vasculitis, pernicious anemia, Sjogren's syndrome, uveitis, psoriasis, Graves ophthalmopathy, alopecia areata, allergic diseases, allergic asthma, atopic dermatitis, allergic rhinitis and/or conjunctivitis, allergic contact dermatitis, inflammatory diseases, inflammatory bowel disease, Crohn's disease, ulcerative colitis, intrinsic asthma, inflammatory lung injury, inflammatory liver injury, inflammatory glomerular injury, atherosclerosis, osteoarthritis, irritant contact dermatitis, eczematous dermatitises, seborrheic dermatitis, cutaneous manifestations of immunologically-mediated disorders, inflammatory eye disease, keratoconjunctivitis, inflammatory myopathy; myocarditis, hepatitis, ischemia/reperfusion injury, myocardial infarction, stroke, gut ischemia, renal failure, hemorrhage shock, traumatic shock, T cell lymphoma, T cell leukemia, infectious diseases, toxic shock, septic shock, adult respiratory distress syndrome, viral infections, AIDS, viral hepatitis, chronic bacterial infection, muscle diseases, polymyositis, senile dementia, pancreatic islet transplant, stem cell transplant, bone marrow transplant, corneal tissue transplant, neuronal tissue transplant, heart transplant, lung transplant, combined heart-lung transplant, kidney transplant, liver transplant, bowel transplant, pancreas transplant, trachea transplant, esophagus transplant, cancer chemotherapy, cancer chemotherapy of solid tumors, breast cancer, peripheral neuropathy, acute demyelinating neuropathies, chronic demyelinating neuropathies, Guillain-Barre syndrome, chronic inflammatory demyelinating polyradiculoneuropathy, multifocal motor neuropathy with conduction block, paraproteinaemic demyelinating peripheral neuropathy, acute inflammatory demyelinating polyneuropathy, polymyositis, dermatomyositis, nerve-muscle diseases, muscular dystrophy, and inclusion body myositis. WO 2004103306; WO 2008000419; WO 2010010127; WO 2010020610; WO 2010071794; WO 2010080409; WO 2010080455; Selmaj et al., Lancet Neurology, 2013, 12, 756-767.

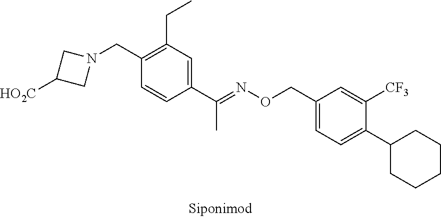 [SMILES: CCc1cc(/C(C)=N/OCc2ccc(C3CCCCC3)c(C(F)(F)F)c2)ccc1CN1CC(C(=O)O)C1]

Siponimod is likely subject to extensive CYP₄₅₀-mediated oxidative metabolism. These, as well as other metabolic transformations, occur in part through polymorphically-expressed enzymes, exacerbating interpatient variability. In order to overcome its short half-life, the drug likely must be taken several times per day, which increases the probability of patient incompliance and discontinuance. Additionally, some metabolites of siponimod may have undesirable side effects. Adverse effects associated with siponimod include decreased heart rate, headache, and dizziness.

### SUMMARY

Provided are deuterium-substituted azetidine compounds, which are modulators of sphingosine 1-phosphate receptor. Also provided are pharmaceutical compositions comprising the deuterium-substituted azetidine compounds, and methods of use thereof, including methods for treating or preventing sphingosine 1-phosphate receptor-mediated disorders by administering, to a patient, the deuterium-substituted azetidine compounds or pharmaceutical compositions comprising the deuterium substituted azetidine compounds. Further provided are methods of synthesizing the deuterium-substituted azetidine compounds.

### DETAILED DESCRIPTION

Before describing several exemplary embodiments of the disclosure, it is to be understood that the disclosure is not limited to the details of construction or process steps set forth in the following description. The disclosure is capable of other embodiments and of being practiced or being carried out in various ways.

All publications and references cited herein are expressly incorporated herein by reference in their entirety. However, with respect to any similar or identical terms found in both the incorporated publications or references and those explicitly put forth or defined in this document, then those terms definitions or meanings explicitly put forth in this document shall control in all respects.

Deuterium Kinetic Isotope Effect

In order to eliminate foreign substances such as therapeutic agents, the animal body expresses various enzymes, such as the cytochrome P₄₅₀ enzymes (CYPs), esterases, proteases, reductases, dehydrogenases, and monoamine oxidases, to react with and convert these foreign substances to more polar intermediates or metabolites for renal excretion. Such metabolic reactions frequently involve the oxidation of a carbon-hydrogen (C—H) bond to either a carbon-oxygen (C—O) or a carbon-carbon (C—C) π-bond. The resultant metabolites may be stable or unstable under physiological conditions, and can have substantially different pharmacokinetic, pharmacodynamic, and acute and long-term toxicity profiles relative to the parent compounds. For most drugs, such oxidations are generally rapid and ultimately lead to administration of multiple or high daily doses.

The relationship between the activation energy and the rate of reaction may be quantified by the Arrhenius equation, k=Ae^{-Eact/RT}. The Arrhenius equation states that, at a given temperature, the rate of a chemical reaction depends exponentially on the activation energy (E_{act}).

The transition state in a reaction is a short lived state along the reaction pathway during which the original bonds have stretched to their limit. By definition, the activation energy E_{act} for a reaction is the energy required to reach the transition state of that reaction. Once the transition state is reached, the molecules can either revert to the original reactants, or form new bonds giving rise to reaction products. A catalyst facilitates a reaction process by lowering the activation energy leading to a transition state. Enzymes are examples of biological catalysts.

Carbon-hydrogen bond strength is directly proportional to the absolute value of the ground-state vibrational energy of the bond. This vibrational energy depends on the mass of the atoms that form the bond, and increases as the mass of one or both of the atoms making the bond increases. Since deuterium (D) has twice the mass of protium (¹H), a C-D bond is stronger than the corresponding C—¹H bond. If a C—¹H bond is broken during a rate-determining step in a chemical reaction (i.e. the step with the highest transition state energy), then substituting a deuterium for that protium will cause a decrease in the reaction rate. This phenomenon is known as the Deuterium Kinetic Isotope Effect (DKIE). The magnitude of the DKIE can be expressed as the ratio between the rates of a given reaction in which a C—¹H bond is broken, and the same reaction where deuterium is substituted for protium. The DKIE can range from about 1 (no isotope effect) to very large numbers, such as 50 or more. Substitution of tritium for hydrogen results in yet a stronger bond than deuterium and gives numerically larger isotope effects

Deuterium (²H or D) is a stable and non-radioactive isotope of hydrogen which has approximately twice the mass of protium (¹H), the most common isotope of hydrogen. Deuterium oxide (D₂O or “heavy water”) looks and tastes like H₂O, but has different physical properties.

When pure D₂O is given to rodents, it is readily absorbed. The quantity of deuterium required to induce toxicity is extremely high. When about 0-15% of the body water has been replaced by D₂O, animals are healthy but are unable to gain weight as fast as the control (untreated) group. When about 15-20% of the body water has been replaced with D₂O, the animals become excitable. When about 20-25% of the body water has been replaced with D₂O, the animals become so excitable that they go into frequent convulsions when stimulated. Skin lesions, ulcers on the paws and muzzles, and necrosis of the tails appear. The animals also become very aggressive. When about 30% of the body water has been replaced with D₂O, the animals refuse to eat and become comatose. Their body weight drops sharply and their metabolic rates drop far below normal, with death occurring at about 30 to about 35% replacement with D₂O. The effects are reversible unless more than thirty percent of the previous body weight has been lost due to D₂O. Studies have also shown that the use of D₂O can delay the growth of cancer cells and enhance the cytotoxicity of certain antineoplastic agents.

Deuteration of pharmaceuticals to improve pharmacokinetics (PK), pharmacodynamics (PD), and toxicity profiles has been demonstrated previously with some classes of drugs. For example, the DKIE was used to decrease the hepatotoxicity of halothane, presumably by limiting the production of reactive species such as trifluoroacetyl chloride. However, this method may not be applicable to all drug classes. For example, deuterium incorporation can lead to metabolic switching. Metabolic switching occurs when xenogens, sequestered by Phase I enzymes, bind transiently and re-bind in a variety of conformations prior to the chemical reaction (e.g., oxidation). Metabolic switching is enabled by the relatively vast size of binding pockets in many Phase I enzymes and the promiscuous nature of many metabolic reactions. Metabolic switching can lead to different proportions of known metabolites as well as altogether new metabolites. This new metabolic profile may impart more or less toxicity. Such pitfalls are non-obvious and are not predictable a priori for any drug class.

Siponimod is a sphingosine 1-phosphate receptor modulator. The carbon-hydrogen bonds of siponimod contain a naturally occurring distribution of hydrogen isotopes, namely ¹H or protium (about 99.9844%), ²H or deuterium (about 0.0156%), and ³H or tritium (in the range between about 0.5 and 67 tritium atoms per 10¹⁸ protium atoms). Increased levels of deuterium incorporation may produce a detectable Deuterium Kinetic Isotope Effect (DKIE) that could affect the pharmacokinetic, pharmacologic and/or toxicologic profiles of such siponimod in comparison with the compound having naturally occurring levels of deuterium.

Based on discoveries made in our laboratory, as well as considering the literature, siponimod is likely metabolized in humans at the azetidine ring, the phenylethyl group, the oxime methyl group, the N- and O-methylene groups, and the cyclohexyl ring. The current approach has the potential to prevent metabolism at these sites. Other sites on the molecule may also undergo transformations leading to metabolites with as-yet-unknown pharmacology/toxicology. Limiting the production of these metabolites has the potential to decrease the danger of the administration of such drugs and may even allow increased dosage and/or increased efficacy. All of these transformations can occur through polymorphically-expressed enzymes, exacerbating interpatient variability. Further, some disorders are best treated when the subject is medicated around the clock or for an extended period of time. For all of the foregoing reasons, a medicine with a longer half-life may result in greater efficacy and cost savings. Various deuteration patterns can be used to (a) reduce or eliminate unwanted metabolites, (b) increase the half-life of the parent drug, (c) decrease the number of doses needed to achieve a desired effect, (d) decrease the amount of a dose needed to achieve a desired effect, (e) increase the formation of active metabolites, if any are formed, (f) decrease the production of deleterious metabolites in specific tissues, and/or (g) create a more effective drug and/or a safer drug for polypharmacy, whether the polypharmacy be intentional or not. The deuteration approach has the strong potential to slow the metabolism of siponimod and attenuate interpatient variability.

Novel compounds and pharmaceutical compositions, certain of which have been found to modulate sphingosine 1-phosphate receptor have been discovered, together with methods of synthesizing and using the compounds, including methods for the treatment of sphingosine 1-phosphate receptor-mediated disorders in a patient by administering the compounds.

Accordingly, provided herein are compounds of structural Formula I:

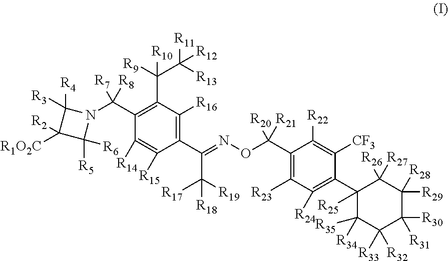

or a salt thereof, wherein:
R₁-R₃₅ are independently selected from the group consisting of hydrogen and deuterium; and
at least one of R₁-R₃₅ is deuterium.

In some aspects, each of R₁-R₃₅ is deuterium. In some aspects, one of R₁-R₃₅ is deuterium and the others are hydrogen. In other aspects, two of R₁-R₃₅ is deuterium and the others are hydrogen. In other aspects, three of R₁-R₃₅ is deuterium and the others are hydrogen. In other aspects, four of R₁-R₃₅ is deuterium and the others are hydrogen. In other aspects, five of R₁-R₃₅ is deuterium and the others are hydrogen. In other aspects, six of R₁-R₃₅ is deuterium and the others are hydrogen. In other aspects, seven of R₁-R₃₅ is deuterium and the others are hydrogen. In other aspects, eight of R₁-R₃₅ is deuterium and the others are hydrogen. In other aspects, nine of R₁-R₃₅ is deuterium and the others are hydrogen. In other aspects, ten of R₁-R₃₅ is deuterium and the others are hydrogen. In other aspects, eleven of R₁-R₃₅ is deuterium and the others are hydrogen. In other aspects, twelve of R₁-R₃₅ is deuterium and the others are hydrogen. In other aspects, thirteen of R₁-R₃₅ is deuterium and the others are hydrogen. In other aspects, fourteen of R₁-R₃₅ is deuterium and the others are hydrogen. In other aspects, fifteen of R₁-R₃₅ is deuterium and the others are hydrogen. In other aspects, sixteen of R₁-R₃₅ is deuterium and the others are hydrogen. In other aspects, seventeen of R₁-R₃₅ is deuterium and the others are hydrogen. In other aspects, eighteen of R₁-R₃₅ is deuterium and the others are hydrogen. In other aspects, nineteen of R₁-R₃₅ is deuterium and the others are hydrogen. In other aspects, twenty of R₁-R₃₅ is deuterium and the others are hydrogen. In other aspects, 21 of R₁-R₃₅ is deuterium and the others are hydrogen. In other aspects, 22 of R₁-R₃₅ is deuterium and the others are hydrogen. In other aspects, 23 of R₁-R₃₅ is deuterium and the others are hydrogen. In other aspects, 24 of R₁-R₃₅ is deuterium and the others are hydrogen. In other aspects, 25 of R₁-R₃₅ is deuterium and the others are hydrogen. In other aspects, 26 of R₁-R₃₅ is deuterium and the others are hydrogen. In other aspects, 27 of R₁-R₃₅ is deuterium and the others are hydrogen. In other aspects, 28 of R₁-R₃₅ is deuterium and the others are hydrogen. In other aspects, 29 of R₁-R₃₅ is deuterium and the others are hydrogen. In other aspects, 30 of R₁-R₃₅ is deuterium and the others are hydrogen. In other aspects, 31 of R₁-R₃₅ is deuterium and the others are hydrogen. In other aspects, 32 of R₁-R₃₅ is deuterium and the others are hydrogen. In other aspects, 33 of R₁-R₃₅ is deuterium and the others are hydrogen. In other aspects, 34 of R₁-R₃₅ is deuterium and the other is hydrogen.

In certain embodiments, R₇ and R₈ are deuterium.

In certain embodiments, R₉ and R₁₀ are deuterium.

In certain embodiments, R₇-R₁₀ are deuterium.

In certain embodiments, R₁₁-R₁₃ are deuterium.

In certain embodiments, R₇, R₈, and R₁₁-R₁₃ are deuterium.

In certain embodiments, R₉-R₁₃ are deuterium.

In certain embodiments, R₇-R₁₃ are deuterium.

In certain embodiments, R₁₇-R₁₉ are deuterium.

In certain embodiments, R₇, R₈, and R₁₇-R₁₉ are deuterium.

In certain embodiments, R₉, R₁₀, and R₁₇-R₁₉ are deuterium.

In certain embodiments, R₇-R₁₀ and R₁₇-R₁₉ are deuterium.

In certain embodiments, R₁₁-R₁₃ and R₁₇-R₁₉ are deuterium.

In certain embodiments, R₇, R₈, R₁₁-R₁₃, and R₁₇-R₁₉ are deuterium.

In certain embodiments, R₉-R₁₃ and R₁₇-R₁₉ are deuterium.

In certain embodiments, R₇-R₁₃ and R₁₇-R₁₉ are deuterium.

In certain embodiments, R₂₀ and R₂₁ are deuterium.

In certain embodiments, R₇, R₈, R₂₀, and R₂₁ are deuterium.

In certain embodiments, R₉, R₁₀, R₂₀, and R₂₁ are deuterium.

In certain embodiments, R₇-R₁₀, R₂₀, and R₂₁ are deuterium.

In certain embodiments, R₁₁-R₁₃, R₂₀, and R₂₁ are deuterium.

In certain embodiments, R₇, R₈, R₁₁-R₁₃, R₂₀, and R₂₁ are deuterium.

In certain embodiments, R₉-R₁₃, R₂₀, and R₂₁ are deuterium.

In certain embodiments, R₇-R₁₃, R₂₀, and R₂₁ are deuterium.

In certain embodiments, R₁₇-R₂₁ are deuterium.

In certain embodiments, R₇, R₈, and R₁₇-R₂₁ are deuterium.

In certain embodiments, R₉, R₁₀, and R₁₇-R₂₁ are deuterium.

In certain embodiments, R₇-R₁₀, and R₁₇-R₂₁ are deuterium.

In certain embodiments, R₁₁-R₁₃ and R₁₇-R₁₉ are deuterium.

In certain embodiments, R₇, R₈, R₁₁-R₁₃, and R₁₇-R₂₁ are deuterium.

In certain embodiments, R₉-R₁₃, and R₁₇-R₂₁ are deuterium.

In certain embodiments, R₇-R₁₃ and R₁₇-R₂₁ are deuterium.

In certain embodiments, R₇-R₁₃ and R₁₇-R₂₁ are deuterium.

In certain embodiments, R₁ is hydrogen.

In certain embodiments, R₁₄-R₁₆ are hydrogen.

In certain embodiments, R₂₂-R₂₄ are hydrogen.

In certain embodiments, R₁, R₁₄-R₁₆, and R₂₂-R₂₄ are hydrogen.

Also provided are further embodiments of any of the above embodiments, wherein R₂ is deuterium.

Also provided are further embodiments of any of the above embodiments, wherein R₃ and R₄ are deuterium.

Also provided are further embodiments of any of the above embodiments, wherein R₂-R₄ are deuterium.

Also provided are further embodiments of any of the above embodiments, wherein R₅ and R₆ are deuterium.

Also provided are further embodiments of any of the above embodiments, wherein R₂, R₅, and R₆ are deuterium.

Also provided are further embodiments of any of the above embodiments, wherein R₃-R₆ are deuterium.

Also provided are further embodiments of any of the above embodiments, wherein R₂-R₆ are deuterium.

Also provided are further embodiments of any of the above embodiments, wherein R₂₅-R₃₅ are deuterium.

Also provided are further embodiments of any of the above embodiments, wherein R₂₆-R₃₅ are deuterium.

In certain embodiments, compounds have structural Formula II:

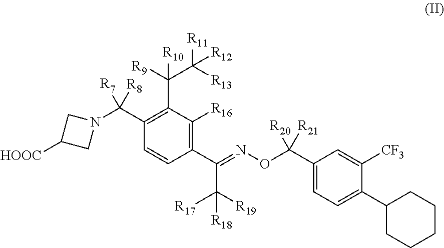

or a salt thereof, wherein:
R₇-R₁₃ and R₁₇-R₂₁ are independently selected from the group consisting of hydrogen and deuterium; and
at least one of R₇-R₁₃ and R₁₇-R₂₁ is deuterium.

In some aspects, each of R₇-R₁₃ and R₁₇-R₂₁ is deuterium. In other aspects, one of R₇-R₁₃ and R₁₇-R₂₁ is deuterium and the others are hydrogen. In other aspects, two of R₇-R₁₃ and R₁₇-R₂₁ is deuterium and the others are hydrogen. In other aspects, three of R₇-R₁₃ and R₁₇-R₂₁ is deuterium and the others are hydrogen. In other aspects, four of R₇-R₁₃ and R₁₇-R₂₁ is deuterium and the others are hydrogen. In other aspects, five of R₇-R₁₃ and R₁₇-R₂₁ is deuterium and the others are hydrogen. In other aspects, six of R₇-R₁₃ and R₁₇-R₂₁ is deuterium and the others are hydrogen. In other aspects, seven of R₇-R₁₃ and R₁₇-R₂₁ is deuterium and the others are hydrogen. In other aspects, eight of R₇-R₁₃ and R₁₇-R₂₁ is deuterium and the others are hydrogen. In other aspects, nine of R₇-R₁₃ and R₁₇-R₂₁ is deuterium and the others are hydrogen. In other aspects, ten of R₇-R₁₃ and R₁₇-R₂₁ is deuterium and the others are hydrogen. In other aspects, eleven of R₇-R₁₃ and R₁₇-R₂₁ is deuterium and the other is hydrogen.

In certain embodiments, R₇ and R₈ are deuterium.

In certain embodiments, R₉ and R₁₀ are deuterium.

In certain embodiments, R₇-R₁₀ are deuterium.

In certain embodiments, R₁₁-R₁₃ are deuterium.

In certain embodiments, R₇, R₈, and R₁₁-R₁₃ are deuterium.

In certain embodiments, R₉-R₁₃ are deuterium.

In certain embodiments, R₇-R₁₃ are deuterium.

In certain embodiments, R₁₇-R₁₉ are deuterium.

In certain embodiments, R₇, R₈, and R₁₇-R₁₉ are deuterium.

In certain embodiments, R₉, R₁₀, and R₁₇-R₁₉ are deuterium.

In certain embodiments, R₇-R₁₀ and R₁₇-R₁₉ are deuterium.

In certain embodiments, R₁₁-R₁₃ and R₁₇-R₁₉ are deuterium.

In certain embodiments, R₇, R₈, R₁₁-R₁₃, and R₁₇-R₁₉ are deuterium.

In certain embodiments, R₉-R₁₃ and R₁₇-R₁₉ are deuterium.

In certain embodiments, R₇-R₁₃ and R₁₇-R₁₉ are deuterium.

In certain embodiments, R₂₀ and R₂₁ are deuterium.

In certain embodiments, R₇, R₈, R₂₀, and R₂₁ are deuterium.

In certain embodiments, R₉, R₁₀, R₂₀, and R₂₁ are deuterium.

In certain embodiments, R₇-R₁₀, R₂₀, and R₂₁ are deuterium.

In certain embodiments, R₁₁-R₁₃, R₂₀, and R₂₁ are deuterium.

In certain embodiments, R₇, R₈, R₁₁-R₁₃, R₂₀, and R₂₁ are deuterium.

In certain embodiments, R₉-R₁₃, R₂₀, and R₂₁ are deuterium.

In certain embodiments, R₇-R₁₃, R₂₀, and R₂₁ are deuterium.

In certain embodiments, R₁₇-R₂₁ are deuterium.

In certain embodiments, R₇, R₈, and R₁₇-R₂₁ are deuterium.

In certain embodiments, R₉, R₁₀, and R₁₇-R₂₁ are deuterium.

In certain embodiments, R₇-R₁₀, and R₁₇-R₂₁ are deuterium.

In certain embodiments, R₁₁-R₁₃ and R₁₇-R₁₉ are deuterium.

In certain embodiments, R₇, R₈, R₁₁-R₁₃, and R₁₇-R₂₁ are deuterium.

In certain embodiments, R₉-R₁₃, and R₁₇-R₂₁ are deuterium.

In certain embodiments, R₇-R₁₃ and R₁₇-R₂₁ are deuterium.

In certain embodiments, R₇-R₁₃ and R₁₇-R₂₁ are deuterium.

Also provided herein are embodiments according to each of the embodiments above, wherein every other substituent among R₁-R₃₅ not specified as deuterium is hydrogen.

Also provided is a compound chosen from the Examples and compounds disclosed herein.

In certain embodiments are provided compounds as disclosed herein, wherein at least one of R₁-R₃₅ independently has deuterium enrichment of no less than about 1%. In certain embodiments are provided compounds as disclosed herein, wherein at least one of R₁-R₃₅ independently has deuterium enrichment of no less than about 10%. In certain embodiments are provided compounds as disclosed herein, wherein at least one of R₁-R₃₅ independently has deuterium enrichment of no less than about 50%. In certain embodiments are provided compounds as disclosed herein, wherein at least one of R₁-R₃₅ independently has deuterium enrichment of no less than about 90%. In certain embodiments are provided compounds as disclosed herein, wherein at least one of R₁-R₃₅ independently has deuterium enrichment of no less than about 95%. In certain embodiments are provided compounds as disclosed herein, wherein at least one of R₁-R₃₅ independently has deuterium enrichment of no less than about 98%.

The compounds as disclosed herein may also contain less prevalent isotopes for other elements, including, but not limited to, ¹³C or ¹⁴C for carbon, ³³S, ³⁴S, or ³⁶S for sulfur, ¹⁵N for nitrogen, and ¹⁷O or ¹⁸O for oxygen.

In certain embodiments, the compound disclosed herein may expose a patient to a maximum of about 0.000005% D₂O or about 0.00001% DHO, assuming that all of the C-D bonds in the compound as disclosed herein are metabolized and released as D₂O or DHO. In certain embodiments, the levels of D₂O shown to cause toxicity in animals is much greater than even the maximum limit of exposure caused by administration of the deuterium enriched compound as disclosed herein. Thus, in certain embodiments, the deuterium-enriched compound disclosed herein should not cause any additional toxicity due to the formation of D₂O or DHO upon drug metabolism.

In certain embodiments are provided compounds as disclosed herein wherein each position represented as D has deuterium enrichment of no less than about 1%. In certain embodiments are provided compounds as disclosed herein wherein each position represented as D has deuterium enrichment of no less than about 10%. In certain embodiments are provided compounds as disclosed herein wherein each position represented as D has deuterium enrichment of no less than about 50%. In certain embodiments are provided compounds as disclosed herein wherein each position represented as D has deuterium enrichment of no less than about 90%. In certain embodiments are provided compounds as disclosed herein wherein each position represented as D has deuterium enrichment of no less than about 95%. In certain embodiments are provided compounds as disclosed herein wherein each position represented as D has deuterium enrichment of no less than about 98%.

In certain embodiments, the deuterated compounds disclosed herein maintain the beneficial aspects of the corresponding non-isotopically enriched molecules while substantially increasing the maximum tolerated dose, decreasing toxicity, increasing the half-life (T_{1/2}), lowering the maximum plasma concentration (Cₘₐₓ) of the minimum efficacious dose (MED), lowering the efficacious dose and thus decreasing the non-mechanism-related toxicity, and/or lowering the probability of drug-drug interactions.

Compounds disclosed herein possess useful sphingosine 1-phosphate receptor modulating activity, and may be used in the treatment or prophylaxis of a disorder in which sphingosine 1-phosphate receptors play an active role. Thus, certain embodiments also provide pharmaceutical compositions comprising one or more compounds disclosed herein together with a pharmaceutically acceptable carrier, as well as methods of making and using the compounds and compositions. Certain embodiments provide methods for modulating sphingosine 1-phosphate receptor. Other embodiments provide methods for treating a sphingosine 1-phosphate receptor-mediated disorder in a patient in need of such treatment, comprising administering to the patient a therapeutically effective amount of a compound or composition according to the present disclosure. Also provided is the use of certain compounds disclosed herein for use in the manufacture of a medicament for the prevention or treatment of a disorder ameliorated by the modulation of sphingosine 1-phosphate receptors.

Also provided is a method of treatment of a sphingosine 1-phosphate receptor-mediated disorder comprising the administration of a therapeutically effective amount of a compound, or a salt thereof, as recited herein to a patient in need thereof.

In certain embodiments, the sphingosine 1-phosphate receptor-mediated disorder is selected from the group consisting of multiple sclerosis, secondary progressive multiple sclerosis, acute or chronic rejection of cell, tissue or organ allo- or xenografts, delayed graft function, graft versus host disease, an autoimmune disease, rheumatoid arthritis, systemic lupus erythematosus, Hashimoto's thyroiditis, myasthenia gravis, diabetes type I, diabetes type II, disorders associated with type I or II diabetes, vasculitis, pernicious anemia, Sjogren's syndrome, uveitis, psoriasis, Graves ophthalmopathy, alopecia areata, an allergic disease, allergic asthma, atopic dermatitis, allergic rhinitis, conjunctivitis, allergic contact dermatitis, an inflammatory disease, inflammatory bowel disease, Crohn's disease, ulcerative colitis, intrinsic asthma, inflammatory lung injury, inflammatory liver injury, inflammatory glomerular injury, atherosclerosis, osteoarthritis, irritant contact dermatitis, eczematous dermatitis, seborrheic dermatitis, a cutaneous manifestation of an immunologically-mediated disorder, inflammatory eye disease, keratoconjunctivitis, inflammatory myopathy, myocarditis, hepatitis, ischemia/reperfusion injury, myocardial infarction, stroke, gut ischemia, renal failure, hemorrhage shock, traumatic shock, T cell lymphoma, T cell leukemia, an infectious disease, toxic shock, septic shock, adult respiratory distress syndrome, a viral infection, AIDS, viral hepatitis, chronic bacterial infection, a muscle disease, polymyositis, senile dementia, pancreatic islet transplant, stem cell transplant, bone marrow transplant, corneal tissue transplant, neuronal tissue transplant, heart transplant, lung transplant, combined heart-lung transplant, kidney transplant, liver transplant, bowel transplant, pancreas transplant, trachea transplant, esophagus transplant, cancer chemotherapy, cancer chemotherapy of a solid tumor, breast cancer, peripheral neuropathy, an acute demyelinating neuropathy, a chronic demyelinating neuropathy, Guillain-Barre syndrome, chronic inflammatory demyelinating polyradiculoneuropathy, multifocal motor neuropathy with conduction block, paraproteinaemic demyelinating peripheral neuropathy, acute inflammatory demyelinating polyneuropathy, polymyositis, dermatomyositis, nerve-muscle diseases, muscular dystrophy, and inclusion body myositis.

In certain embodiments, the disorder is multiple sclerosis.

In certain embodiments, the method of treatment of a sphingosine 1-phosphate receptor-mediated disorder further comprises the administration of an additional therapeutic agent.

In certain embodiments, the additional therapeutic agent is selected from the group consisting of a glucocorticoid and an immunosuppressant.

In certain embodiments, the glucocorticoid is selected from the group consisting of beclometasone, budesonide, flunisolide, betamethasone, fluticasone, triamcinolone, mometasone, ciclesonide, hydrocortisone, cortisone acetate, prednisone, prednisolone, methylprednisolone, and dexamethasone.

In certain embodiments, the immunosuppressant is selected from the group consisting of CP-690550, fingolimod, cyclosporine A, azathioprine, dexamethasone, tacrolimus, sirolimus, pimecrolimus, mycophenolate salts, everolimus, basiliximab, daclizumab, anti-thymocyte globulin, anti-lymphocyte globulin, and CTLA4IgG.

In certain embodiments, the method of treatment of a sphingosine 1-phosphate receptor-mediated disorder further results in at least one effect selected from the group consisting of:
a) decreased inter-individual variation in plasma levels of the compound or a metabolite thereof as compared to the non-isotopically enriched compound;
b) increased average plasma levels of the compound per dosage unit thereof as compared to the non-isotopically enriched compound;
c) decreased average plasma levels of at least one metabolite of the compound per dosage unit thereof as compared to the non-isotopically enriched compound;
d) increased average plasma levels of at least one metabolite of the compound per dosage unit thereof as compared to the non-isotopically enriched compound; and
e) an improved clinical effect during the treatment in the subject per dosage unit thereof as compared to the non-isotopically enriched compound.

In certain embodiments, the method of treatment of a sphingosine 1-phosphate receptor-mediated disorder further results in at least two effects selected from the group consisting of:
a) decreased inter-individual variation in plasma levels of the compound or a metabolite thereof as compared to the non-isotopically enriched compound;
b) increased average plasma levels of the compound per dosage unit thereof as compared to the non-isotopically enriched compound;
c) decreased average plasma levels of at least one metabolite of the compound per dosage unit thereof as compared to the non-isotopically enriched compound;
d) increased average plasma levels of at least one metabolite of the compound per dosage unit thereof as compared to the non-isotopically enriched compound; and
e) an improved clinical effect during the treatment in the subject per dosage unit thereof as compared to the non-isotopically enriched compound.

In certain embodiments, the method effects a decreased metabolism of the compound per dosage unit thereof by at least one polymorphically-expressed cytochrome P₄₅₀ isoform in the subject, as compared to the corresponding non-isotopically enriched compound.

In certain embodiments, the cytochrome P₄₅₀ isoform is selected from the group consisting of CYP2C8, CYP2C9, CYP2C19, and CYP2D6.

In certain embodiments, the compound is characterized by decreased inhibition of at least one cytochrome P₄₅₀ or monoamine oxidase isoform in the subject per dosage unit thereof as compared to the non-isotopically enriched compound.

In certain embodiments, the cytochrome P₄₅₀ or monoamine oxidase isoform is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, CYP51, MAO_{A}, and MAO_{B}.

In certain embodiments, the method reduces a deleterious change in a diagnostic hepatobiliary function endpoint, as compared to the corresponding non-isotopically enriched compound.

In certain embodiments, the diagnostic hepatobiliary function endpoint is selected from the group consisting of alanine aminotransferase (“ALT”), serum glutamic-pyruvic transaminase (“SGPT”), aspartate aminotransferase (“AST,” “SGOT”), ALT/AST ratios, serum aldolase, alkaline phosphatase (“ALP”), ammonia levels, bilirubin, gamma-glutamyl transpeptidase (“GGTP,” “γ-GTP,” “GGT”), leucine aminopeptidase (“LAP”), liver biopsy, liver ultrasonography, liver nuclear scan, 5′-nucleotidase, and blood protein.

Also provided is a compound, or a salt thereof, as recited herein for use as a medicament.

Also provided is a compound, or a salt thereof, as recited herein for use in the manufacture of a medicament for the prevention or treatment of a sphingosine 1-phosphate receptor-mediated disorder.

All publications and references cited herein are expressly incorporated herein by reference in their entirety. However, with respect to any similar or identical terms found in both the incorporated publications or references and those explicitly put forth or defined in this document, then those terms definitions or meanings explicitly put forth in this document shall control in all respects.

As used herein, the terms below have the meanings indicated.

The singular forms “a,” “an,” and “the” may refer to plural articles unless specifically stated otherwise.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

When ranges of values are disclosed, and the notation “from n₁ . . . to n₂” or “n₁-n₂” is used, where n₁ and n₂ are the numbers, then unless otherwise specified, this notation is intended to include the numbers themselves and the range between them. This range may be integral or continuous between and including the end values.

The term “deuterium enrichment” refers to the percentage of incorporation of deuterium at a given position in a molecule in the place of hydrogen. For example, deuterium enrichment of 1% at a given position means that 1% of molecules in a given sample contain deuterium at the specified position. Because the naturally occurring distribution of deuterium is about 0.0156%, deuterium enrichment at any position in a compound synthesized using non-enriched starting materials is about 0.0156%. The deuterium enrichment can be determined using conventional analytical methods known to one of ordinary skill in the art, including mass spectrometry and nuclear magnetic resonance spectroscopy.

The term “is/are deuterium,” when used to describe a given position in a molecule such as R₁-R₃₅ or the symbol “D,” when used to represent a given position in a drawing of a molecular structure, means that the specified position is enriched with deuterium above the naturally occurring distribution of deuterium. The same is true of the term “contains deuterium,” which is often used to refer to methyl groups which may be mono-, di- or trideuterated (e.g., such groups may be —CH₂D, —CD₂H, and —CD₃, wherein the each position denoted D is enriched with deuterium above the naturally occurring distribution of deuterium). In one embodiment deuterium enrichment is no less than about 1%, in another no less than about 5%, in another no less than about 10%, in another no less than about 20%, in another no less than about 50%, in another no less than about 70%, in another no less than about 80%, in another no less than about 90%, or in another no less than about 98% of deuterium at the specified position.

The term “isotopic enrichment” refers to the percentage of incorporation of a less prevalent isotope of an element at a given position in a molecule in the place of the more prevalent isotope of the element.

The term “non-isotopically enriched” refers to a molecule in which the percentages of the various isotopes are substantially the same as the naturally occurring percentages.

Asymmetric centers exist in the compounds disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present disclosure includes all cis, trans, syn, anti, entgegen (E), and zusammen (Z) isomers as well as the appropriate mixtures thereof. Additionally, compounds may exist as tautomers; all tautomeric isomers are provided by this disclosure. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disorder” as used herein is intended to be generally synonymous, and is used interchangeably with, the terms “disease” and “condition” (as in medical condition), in that all reflect an abnormal condition of the human or animal body or of one of its parts that impairs normal functioning, is typically manifested by distinguishing signs and symptoms.

The terms “treat,” “treating,” and “treatment” are meant to include alleviating or abrogating a disorder or one or more of the symptoms associated with a disorder; or alleviating or eradicating the cause(s) of the disorder itself. As used herein, reference to “treatment” of a disorder is intended to include prevention. The terms “prevent,” “preventing,” and “prevention” refer to a method of delaying or precluding the onset of a disorder; and/or its attendant symptoms, barring a subject from acquiring a disorder or reducing a subject's risk of acquiring a disorder.

The term “therapeutically effective amount” refers to the amount of a compound that, when administered, is sufficient to prevent development of, or alleviate to some extent, one or more of the symptoms of the disorder being treated. The term “therapeutically effective amount” also refers to the amount of a compound that is sufficient to elicit the biological or medical response of a cell, tissue, system, animal, or human that is being sought by a researcher, veterinarian, medical doctor, or clinician.

The term “subject” refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein in reference, for example, to a mammalian subject, such as a human patient.

The term “combination therapy” means the administration of two or more therapeutic agents to treat a therapeutic disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the disorders described herein.

The term “sphingosine 1-phosphate receptor” refers to a family of cell surface receptors which bind sphingosine 1-phosphate, of which five subtypes are known (S1P₁₋₅). It appears that ligand binding at each receptor subtype activates a different intracellular signaling pathway. Either S1P-dependent signaling or direct intracellular action of S1P has been implicated in the regulation of a number of physiological processes, of which the best substantiated involve trafficking of lymphocytes and modulation of heart rate and vascular tone.

The term “sphingosine 1-phosphate receptor-mediated disorder,” refers to a disorder that is characterized by abnormal sphingosine 1-phosphate receptor activity. A sphingosine 1-phosphate receptor-mediated disorder may be completely or partially mediated by modulating sphingosine 1-phosphate receptors. In particular, a sphingosine 1-phosphate receptor-mediated disorder is one in which modulation of sphingosine 1-phosphate receptors results in some effect on the underlying disorder e.g., administration of a sphingosine 1-phosphate receptor modulator results in some improvement in at least some of the patients being treated.

The term “sphingosine 1-phosphate receptor modulator,” refers to the ability of a compound disclosed herein to alter the function of sphingosine 1-phosphate receptors. A modulator may activate the activity of a sphingosine 1-phosphate receptor, may activate or inhibit the activity of a sphingosine 1-phosphate receptor depending on the concentration of the compound exposed to the sphingosine 1-phosphate receptor, or may inhibit the activity of a sphingosine 1-phosphate receptor. Such activation or inhibition may be contingent on the occurrence of a specific event, such as activation of a signal transduction pathway, and/or may be manifest only in particular cell types. The term “modulate” or “modulation” also refers to altering the function of a sphingosine 1-phosphate receptor by increasing or decreasing the probability that a complex forms between a sphingosine 1-phosphate receptor and a natural binding partner. A modulator may increase the probability that such a complex forms between the sphingosine 1-phosphate receptor and the natural binding partner, may increase or decrease the probability that a complex forms between the sphingosine 1-phosphate receptor and the natural binding partner depending on the concentration of the compound exposed to the sphingosine 1-phosphate receptor, and or may decrease the probability that a complex forms between the sphingosine 1-phosphate receptor and the natural binding partner. In some embodiments, modulation of the sphingosine 1-phosphate receptor may be assessed using the techniques described in WO 2010020610, WO 2010010127, WO 2004103306, Gergely et al., Brit. J. Pharmacol., 2012, 167, 1035-47, the disclosures of which are incorporated herein by reference in its entirety.

The term “therapeutically acceptable” refers to those compounds (or salts, prodrugs, tautomers, zwitterionic forms, etc.) which are suitable for use in contact with the tissues of patients without excessive toxicity, irritation, allergic response, immunogenecity, are commensurate with a reasonable benefit/risk ratio, and are effective for their intended use.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Each component must be “pharmaceutically acceptable” in the sense of being compatible with the other ingredients of a pharmaceutical formulation. It must also be suitable for use in contact with the tissue or organ of humans and animals without excessive toxicity, irritation, allergic response, immunogenecity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004).

The terms “active ingredient,” “active compound,” and “active substance” refer to a compound, which is administered, alone or in combination with one or more pharmaceutically acceptable excipients or carriers, to a subject for treating, preventing, or ameliorating one or more symptoms of a disorder.

The terms “drug,” “therapeutic agent,” and “chemotherapeutic agent” refer to a compound, or a pharmaceutical composition thereof, which is administered to a subject for treating, preventing, or ameliorating one or more symptoms of a disorder.

The term “release controlling excipient” refers to an excipient whose primary function is to modify the duration or place of release of the active substance from a dosage form as compared with a conventional immediate release dosage form.

The term “nonrelease controlling excipient” refers to an excipient whose primary function do not include modifying the duration or place of release of the active substance from a dosage form as compared with a conventional immediate release dosage form.

The term “prodrug” refers to a compound functional derivative of the compound as disclosed herein and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis.

Prodrugs may include esters of carboxylic acids, such as, for example, compounds of Formula Ia:

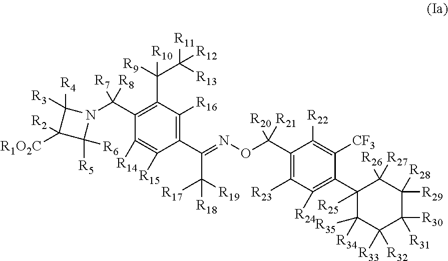

or a salt thereof, wherein:

R₁ is chosen from methyl, ethyl, n-propyl, isopropyl, n-butyl, isobutyl, tert-butyl, pentyl, and hexyl; and

R₂-R₃₅ are independently selected from the group consisting of hydrogen and deuterium;

at least one of R₂-R₃₅ is deuterium or contains deuterium.

The compounds disclosed herein can exist as therapeutically acceptable salts. The term “therapeutically acceptable salt,” as used herein, represents salts or zwitterionic forms of the compounds disclosed herein which are therapeutically acceptable as defined herein. The salts can be prepared during the final isolation and purification of the compounds or separately by reacting the appropriate compound with a suitable acid or base. Therapeutically acceptable salts include acid and basic addition salts.

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, a-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

While it may be possible for the compounds of the subject disclosure to be administered as the raw chemical, it is also possible to present them as a pharmaceutical composition. Accordingly, provided herein are pharmaceutical compositions which comprise one or more of certain compounds disclosed herein, or one or more pharmaceutically acceptable salts, prodrugs, or solvates thereof, together with one or more pharmaceutically acceptable carriers thereof and optionally one or more other therapeutic ingredients. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art.

The compositions include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The compositions may conveniently be presented in unit dosage form and may be prepared by any of the methods well known in the art of pharmacy. Typically, these methods include the step of bringing into association a compound of the subject disclosure or a pharmaceutically salt, prodrug, or solvate thereof (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose.

For administration by inhalation, compounds may be delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the disclosure may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the disorder being treated. Also, the route of administration may vary depending on the disorder and its severity.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disorder.

In the case wherein the patient's status does improve, upon the doctor's discretion the administration of the compounds may be given continuously or temporarily suspended for a certain length of time (i.e., a “drug holiday”).

Once improvement of the patient's conditions has occurred, a maintenance dose is administered if necessary. Subsequently, the dosage or the frequency of administration, or both, can be reduced, as a function of the symptoms, to a level at which the improved disorder is retained. Patients can, however, require intermittent treatment on a long-term basis upon any recurrence of symptoms.

Disclosed herein are methods of treating a sphingosine 1-phosphate receptor-mediated disorder comprising administering to a subject having or suspected to have such a disorder, a therapeutically effective amount of a compound as disclosed herein or a pharmaceutically acceptable salt, solvate, or prodrug thereof.

Sphingosine 1-phosphate receptor-mediated disorders, include, but are not limited to, multiple sclerosis, secondary progressive multiple sclerosis, acute or chronic rejection of cell, tissue or organ allo- or xenografts, delayed graft function, graft versus host disease, autoimmune diseases, rheumatoid arthritis, systemic lupus erythematosus, Hashimoto's thyroiditis, myasthenia gravis, diabetes type I or II and the disorders associated therewith, vasculitis, pernicious anemia, Sjogren's syndrome, uveitis, psoriasis, Graves ophthalmopathy, alopecia areata, allergic diseases, allergic asthma, atopic dermatitis, allergic rhinitis and/or conjunctivitis, allergic contact dermatitis, inflammatory diseases, inflammatory bowel disease, Crohn's disease, ulcerative colitis, intrinsic asthma, inflammatory lung injury, inflammatory liver injury, inflammatory glomerular injury, atherosclerosis, osteoarthritis, irritant contact dermatitis, eczematous dermatitises, seborrheic dermatitis, cutaneous manifestations of immunologically-mediated disorders, inflammatory eye disease, keratoconjunctivitis, inflammatory myopathy; myocarditis, hepatitis, ischemia/reperfusion injury, myocardial infarction, stroke, gut ischemia, renal failure, hemorrhage shock, traumatic shock, T cell lymphoma, T cell leukemia, infectious diseases, toxic shock, septic shock, adult respiratory distress syndrome, viral infections, AIDS, viral hepatitis, chronic bacterial infection, muscle diseases, polymyositis, senile dementia, pancreatic islet transplant, stem cell transplant, bone marrow transplant, corneal tissue transplant, neuronal tissue transplant, heart transplant, lung transplant, combined heart-lung transplant, kidney transplant, liver transplant, bowel transplant, pancreas transplant, trachea transplant, esophagus transplant, cancer chemotherapy, cancer chemotherapy of solid tumors, breast cancer, peripheral neuropathy, acute demyelinating neuropathies, chronic demyelinating neuropathies, Guillain-Barre syndrome, chronic inflammatory demyelinating polyradiculoneuropathy, multifocal motor neuropathy with conduction block, paraproteinaemic demyelinating peripheral neuropathy, acute inflammatory demyelinating polyneuropathy, polymyositis, dermatomyositis, nerve-muscle diseases, muscular dystrophy, and inclusion body myositis, and/or any disorder which can lessened, alleviated, or prevented by administering a sphingosine 1-phosphate receptor modulator.

In certain embodiments, a method of treating a sphingosine 1-phosphate receptor-mediated disorder comprises administering to the subject a therapeutically effective amount of a compound of as disclosed herein, or a pharmaceutically acceptable salt, solvate, or prodrug thereof, so as to affect: (1) decreased inter-individual variation in plasma levels of the compound or a metabolite thereof; (2) increased average plasma levels of the compound or decreased average plasma levels of at least one metabolite of the compound per dosage unit; (3) decreased inhibition of, and/or metabolism by at least one cytochrome P₄₅₀ or monoamine oxidase isoform in the subject; (4) decreased metabolism via at least one polymorphically-expressed cytochrome P₄₅₀ isoform in the subject; (5) at least one statistically-significantly improved disorder-control and/or disorder-eradication endpoint; (6) an improved clinical effect during the treatment of the disorder, (7) prevention of recurrence, or delay of decline or appearance, of abnormal alimentary or hepatic parameters as the primary clinical benefit, or (8) reduction or elimination of deleterious changes in any diagnostic hepatobiliary function endpoints, as compared to the corresponding non-isotopically enriched compound.

In certain embodiments, inter-individual variation in plasma levels of the compounds as disclosed herein, or metabolites thereof, is decreased; average plasma levels of the compound as disclosed herein are increased; average plasma levels of a metabolite of the compound as disclosed herein are decreased; inhibition of a cytochrome P₄₅₀ or monoamine oxidase isoform by a compound as disclosed herein is decreased; or metabolism of the compound as disclosed herein by at least one polymorphically-expressed cytochrome P₄₅₀ isoform is decreased; by greater than about 5%, greater than about 10%, greater than about 20%, greater than about 30%, greater than about 40%, or by greater than about 50% as compared to the corresponding non-isotopically enriched compound.

Plasma levels of the compound as disclosed herein, or metabolites thereof, may be measured using the methods described in Gergely et al., Brit. J. Pharmacol., 2012, 167, 1035-47, which is hereby incorporated by reference.

Examples of cytochrome P₄₅₀ isoforms in a mammalian subject include, but are not limited to, CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

Examples of monoamine oxidase isoforms in a mammalian subject include, but are not limited to, MAO_{A}, and MAO_{B}.

The inhibition of the cytochrome P₄₅₀ isoform is measured by the method of Ko et al. (British Journal of Clinical Pharmacology, 2000, 49, 343-351). The inhibition of the MAO_{A} isoform is measured by the method of Weyler et al. (J. Biol Chem. 1985, 260, 13199-13207). The inhibition of the MAO_{B} isoform is measured by the method of Uebelhack et al. (Pharmacopsychiatry, 1998, 31, 187-192).

Examples of polymorphically-expressed cytochrome P₄₅₀ isoforms in a mammalian subject include, but are not limited to, CYP2C8, CYP2C9, CYP2C19, and CYP2D6.

The metabolic activities of liver microsomes, cytochrome P₄₅₀ isoforms, and monoamine oxidase isoforms are measured by the methods described herein.

Examples of improved disorder-control and/or disorder-eradication endpoints, or improved clinical effects include, but are not limited to, delay in time to confirmed disability progression, expanded disability status scale score, worsening of 25 foot walk test, increase in T2 lesion volume, annualized relapse rate, time to the first relapse, twelve item MS walking scale, inflammatory disease activity, and burden of disease as measured by MRI.

Examples of diagnostic hepatobiliary function endpoints include, but are not limited to, alanine aminotransferase (“ALT”), serum glutamic-pyruvic transaminase (“SGPT”), aspartate aminotransferase (“AST” or “SGOT”), ALT/AST ratios, serum aldolase, alkaline phosphatase (“ALP”), ammonia levels, bilirubin, gamma-glutamyl transpeptidase (“GGTP,” “γ-GTP,” or “GGT”), leucine aminopeptidase (“LAP”), liver biopsy, liver ultrasonography, liver nuclear scan, 5′-nucleotidase, and blood protein. Hepatobiliary endpoints are compared to the stated normal levels as given in “Diagnostic and Laboratory Test Reference”, 4^{th} edition, Mosby, 1999. These assays are run by accredited laboratories according to standard protocol.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### Combination Therapy

The compounds disclosed herein may also be combined or used in combination with other agents useful in the treatment of sphingosine 1-phosphate receptor-mediated disorders. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with a compound as disclosed herein. When a compound as disclosed herein is used contemporaneously with one or more other drugs, a pharmaceutical composition containing such other drugs in addition to the compound disclosed herein may be utilized, but is not required.

In certain embodiments, the compounds disclosed herein can be combined with one or more glucocorticoids and immunosuppressants.

In certain embodiments, the compounds disclosed herein can be combined with one or more glucocorticoids selected from the group consisting of beclometasone, budesonide, flunisolide, betamethasone, fluticasone, triamcinolone, mometasone, ciclesonide, hydrocortisone, cortisone acetate, prednisone, prednisolone, methylprednisolone, and dexamethasone.

In certain embodiments, the compounds disclosed herein can be combined with one or more immunosuppressants selected from the group consisting of CP-690550, fingolimod, cyclosporine A, azathioprine, dexamethasone, tacrolimus, sirolimus, pimecrolimus, mycophenolate salts, everolimus, basiliximab, daclizumab, anti-thymocyte globulin, anti-lymphocyte globulin, and CTLA4IgG.

The compounds disclosed herein can also be administered in combination with other classes of compounds, including, but not limited to, norepinephrine reuptake inhibitors (NRIs) such as atomoxetine; dopamine reuptake inhibitors (DARIs), such as methylphenidate; serotonin-norepinephrine reuptake inhibitors (SNRIs), such as milnacipran; sedatives, such as diazepham; norepinephrine-dopamine reuptake inhibitor (NDRIs), such as bupropion; serotonin-norepinephrine-dopamine-reuptake-inhibitors (SNDRIs), such as venlafaxine; monoamine oxidase inhibitors, such as selegiline; hypothalamic phospholipids; endothelin converting enzyme (ECE) inhibitors, such as phosphoramidon; opioids, such as tramadol; thromboxane receptor antagonists, such as ifetroban; potassium channel openers; thrombin inhibitors, such as hirudin; hypothalamic phospholipids; growth factor inhibitors, such as modulators of PDGF activity; platelet activating factor (PAF) antagonists; anti-platelet agents, such as GPIIb/IIIa blockers (e.g., abdximab, eptifibatide, and tirofiban), P2Y(AC) antagonists (e.g., clopidogrel, ticlopidine and CS-747), and aspirin; anticoagulants, such as warfarin; low molecular weight heparins, such as enoxaparin; Factor VIIa Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, or atavastatin or visastatin); squalene synthetase inhibitors; fibrates; bile acid sequestrants, such as questran; niacin; anti-atherosclerotic agents, such as ACAT inhibitors; MTP Inhibitors; calcium channel blockers, such as amlodipine besylate; potassium channel activators; alpha-muscarinic agents; beta-muscarinic agents, such as carvedilol and metoprolol; antiarrhythmic agents; diuretics, such as chlorothlazide, hydrochiorothiazide, flumethiazide, hydroflumethiazide, bendroflumethiazide, methylchlorothiazide, trichioromethiazide, polythiazide, benzothlazide, ethacrynic acid, tricrynafen, chlorthalidone, furosenilde, musolimine, bumetanide, triamterene, amiloride, and spironolactone; thrombolytic agents, such as tissue plasminogen activator (tPA), recombinant tPA, streptokinase, urokinase, prourokinase, and anisoylated plasminogen streptokinase activator complex (APSAC); anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; antiinflammatories; antiproliferatives, such as methotrexate, FK506 (tacrolimus, Prograf), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, and octreotide acetate; microtubule-disruptor agents, such as ecteinascidins; microtubule-stabilizing agents, such as pacitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporins; steroids, such as prednisone and dexamethasone; cytotoxic drugs, such as azathiprine and cyclophosphamide; TNF-alpha inhibitors, such as tenidap; anti-TNF antibodies or soluble TNF receptor, such as etanercept, rapamycin, and leflunimide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin.

Thus, in another aspect, certain embodiments provide methods for treating sphingosine 1-phosphate receptor-mediated disorders in a human or animal subject in need of such treatment comprising administering to the subject an amount of a compound disclosed herein effective to reduce or prevent the disorder in the subject, in combination with at least one additional agent for the treatment of the disorder that is known in the art. In a related aspect, certain embodiments provide therapeutic compositions comprising at least one compound disclosed herein in combination with one or more additional agents for the treatment of sphingosine 1-phosphate receptor-mediated disorders.

### General Synthetic Methods for Preparing Compounds

Isotopic hydrogen can be introduced into a compound as disclosed herein by synthetic techniques that employ deuterated reagents, whereby incorporation rates are pre-determined; and/or by exchange techniques, wherein incorporation rates are determined by equilibrium conditions, and may be highly variable depending on the reaction conditions. Synthetic techniques, where tritium or deuterium is directly and specifically inserted by tritiated or deuterated reagents of known isotopic content, may yield high tritium or deuterium abundance, but can be limited by the chemistry required. Exchange techniques, on the other hand, may yield lower tritium or deuterium incorporation, often with the isotope being distributed over many sites on the molecule.

The compounds as disclosed herein can be prepared by methods known to one of skill in the art and routine modifications thereof, and/or following procedures similar to those described in the Example section herein and routine modifications thereof, and/or procedures found in WO 2004103306, which is hereby incorporated in their entirety, and references cited therein and routine modifications thereof. Compounds as disclosed herein can also be prepared as shown in any of the following schemes and routine modifications thereof.

The following schemes can be used to practice the present disclosure. Any position shown as hydrogen may optionally be replaced with deuterium.

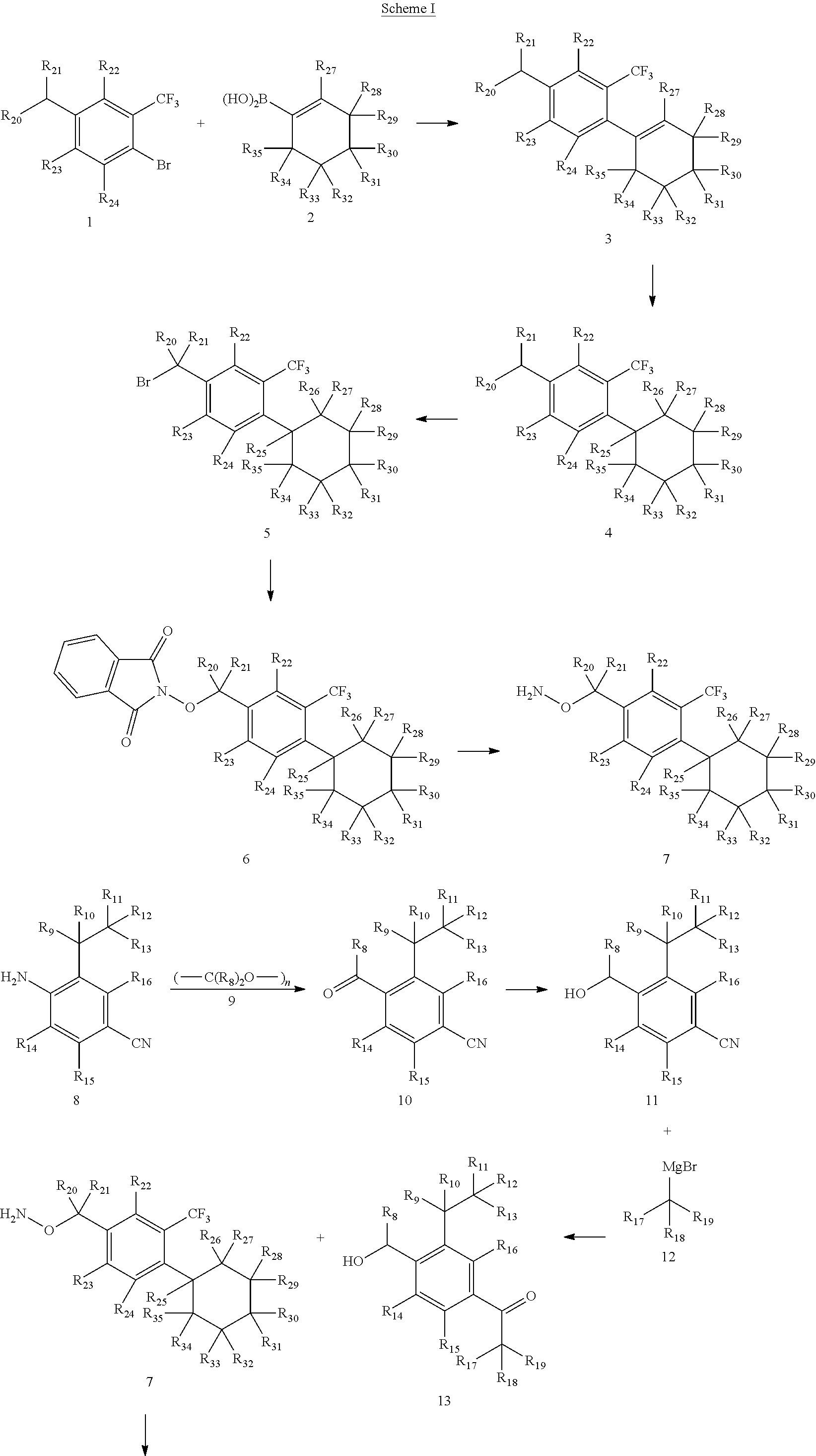

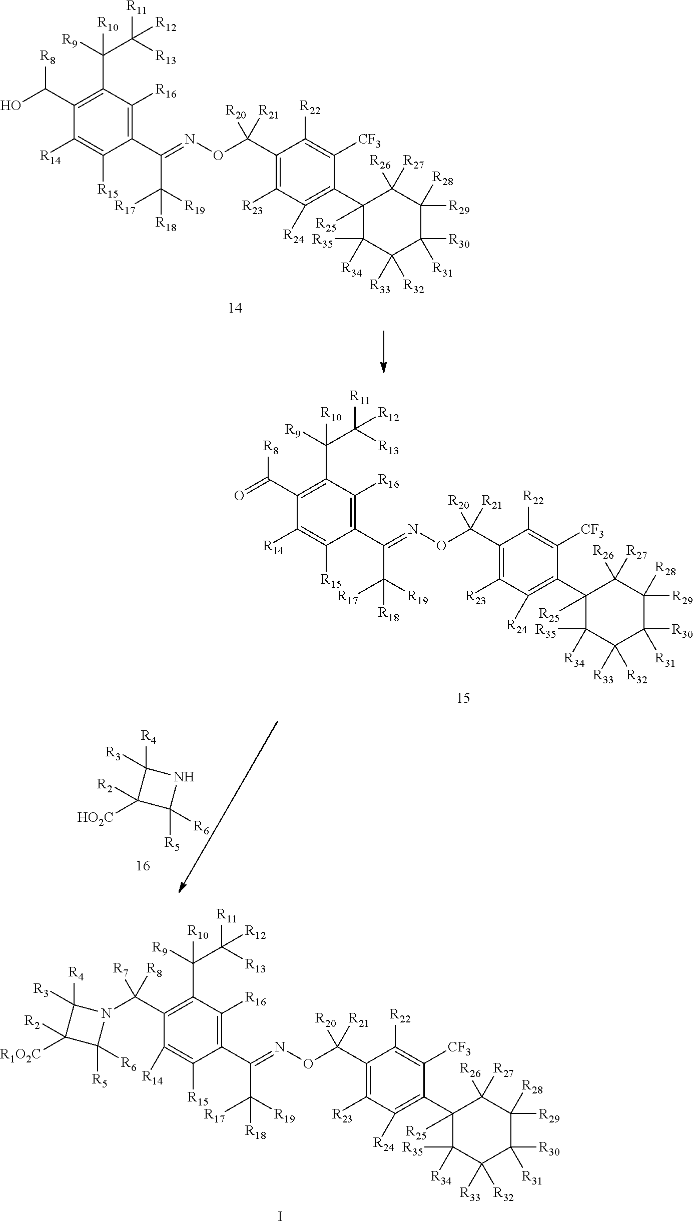

Compound 1 is reacted with compound 2 in the presence of an appropriate catalyst, such as bis-triphenylphosphine palladium dichloride, in the presence of an appropriate base, such as sodium methoxide, an appropriate solvent, such as methanol, to give compound 3. Compound 3 is treated with an appropriate reducing agent, such as a combination of palladium on carbon and hydrogen gas, in an appropriate solvent, such as methanol, to give compound 4. Compound 4 is treated with an appropriate brominating agent, such as a combination of N-bromosuccinimide and azobisisobutyronitrile, in an appropriate solvent, such as acetonitrile, at an elevated temperature, to give compound 5. Compound 5 is treated with an appropriate hydroxylamine source, such as N-hydroxysuccinimide, in the presence of an appropriate base, such as potassium carbonate, in an appropriate solvent, such as dimethyl sulfoxide, to give compound 6. Compound 6 is treated with an appropriate deprotecting agent, such as hydrazine hydrate, in an appropriate solvent, such as ethanol, at an elevated temperature, to give compound 7. Compound 8 is treated with an nitrite source, such as sodium nitrite, in the presence of an appropriate acid, such as hydrochloric acid, and then reacted with compound 9 in the presence of an appropriate catalyst, such as a combination of cupric sulfate, sodium sulfite, and sodium acetate, in an appropriate solvent, such as water, to give compound 10. Compound 10 is treated with an appropriate reducing agent, such as sodium borohydride, in an appropriate solvent, such as ethanol, to give compound 11. Compound 11 is reacted with compound 12 in an appropriate solvent, such as tetrahydrofuran, to give compound 13. Compound 13 is reacted with compound 7 in the presence of an appropriate acid, such as acetic acid, in an appropriate solvent, such as methanol, to give compound 14. Compound 14 is treated with an appropriate oxidizing agent, such as manganese dioxide, in an appropriate solvent, such as 1,4-dioxane, at an elevated temperature, to give compound 15. Compound 15 is reacted with compound 16 in the presence of an appropriate base, such as triethylamine, in the presence of an appropriate reducing agent, such as sodium cyanoborohydride, in an appropriate solvent, such as methanol, to give a compound of formula I.

Deuterium can be incorporated to different positions synthetically, according to the synthetic procedures as shown in Scheme I, by using appropriate deuterated intermediates. For example, to introduce deuterium at R₂₀-R₂₄, compound 1 with the corresponding deuterium substitutions can be used. To introduce deuterium at R₂₇-R₃₅, compound 2 with the corresponding deuterium substitutions can be used. To introduce deuterium at R₂₅-R₂₆, deuterium gas can be used. To introduce deuterium at R₉-R₁₆, compound 8 with the corresponding deuterium substitutions can be used. To introduce deuterium at R₈, compound 9 with the corresponding deuterium substitutions can be used. To introduce deuterium at one or more positions of R₁₇-R₁₉, compound 12 with the corresponding deuterium substitutions can be used. To introduce deuterium at one or more positions of R₂-R₆, compound 16 with the corresponding deuterium substitutions can be used. To introduce deuterium at R₇, sodium cyanoborodeuteride can be used.

Deuterium can be incorporated to various positions having an exchangeable proton, such as the carboxyl O—H, via proton-deuterium equilibrium exchange. For example, to introduce deuterium at R₁ this proton may be replaced with deuterium selectively or non-selectively through a proton-deuterium exchange method known in the art.

The disclosure is now described with reference to the following examples. Before describing several exemplary embodiments of the disclosure, it is to be understood that the disclosure is not limited to the details of construction or process steps set forth in the following description. The disclosure is capable of other embodiments and of being practiced or being carried out in various ways.

The following abbreviations may be employed in the Examples and elsewhere herein:

DMA=dimethylacetamide

DMF=dimethylformamide

DMSO=dimethyl sulfoxide

DCM=dichloromethane

THF=tetrahydrofuran

TEA=triethylamine

LDA=lithium diisopropylamide

IBX=2-iodoxy benzoic acid

TosCl=4-toluene sulfonyl chloride

TBAC=tert-butyl acetate

L=liter

mL=milliliter

μL-microliter

g=gram(s)

mg=milligram(s)

mol=moles

mmol=millimole(s)

h or hr=hour(s)

min=minute(s)

Equiv=equivalent(s)

H₂=hydrogen

Ar=argon

N₂=nitrogen

RT or R.T.=room temperature

AT=ambient temperature

Aq.=aqueous

HPLC=high performance liquid chromatography

HPLC R,=HPLC retention time

LC/MS=high performance liquid chromatography/mass spectrometry

MS or Mass Spec=mass spectrometry

NMR=nuclear magnetic resonance

NMR spectral data: s=singlet; d=doublet; m=multiplet; br=broad; t=triplet

mp=melting point

All IUPAC names were generated using PerkinElmer®'s ChemDraw.

### EXAMPLES

### Example 1—Comparative

### 1-([4-[(1E)-1-([[4-cyclohexyl-3-(trifluoromethyl)phenyl]methoxy]imino)ethyl]-2-ethylphenyl]methyl)azetidine-3-carboxylic acid (Siponimod)

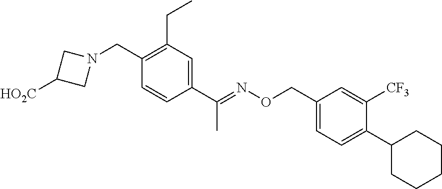 [SMILES: CCc1cc(/C(C)=N/OCc2ccc(C3CCCCC3)c(C(F)(F)F)c2)ccc1CN1CC(C(=O)O)C1]

Step 1

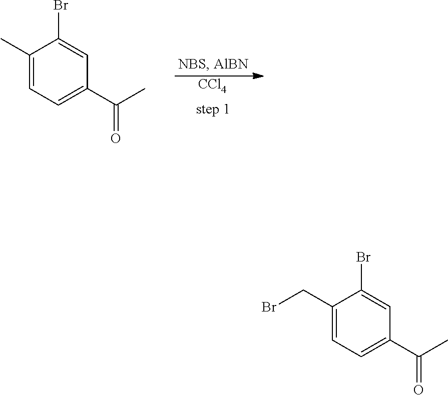

1-[3-bromo-4-(bromomethyl)phenyl]ethan-1-one: To a solution of 1-(3-bromo-4-methylphenyl)ethan-1-one (30 g, 140.80 mmol, 1.00 equiv.) in CCl₄ (300 mL), NBS (32.75 g, 184.01 mmol, 1.30 equiv.) and AIBN (4.635 g, 28.23 mmol, 0.20 equiv.) were added. The resulting solution was stirred for 12 h at 80° C. The solids were filtered out. The resulting solution was extracted with DCM (2×300 mL). The organic layers were combined, dried over anhydrous sodium sulfate and concentrated under vacuum to afford 28.95 g (70%) of 1-[3-bromo-4-(bromomethyl)phenyl]ethan-1-one as light yellow oil.

Step 2

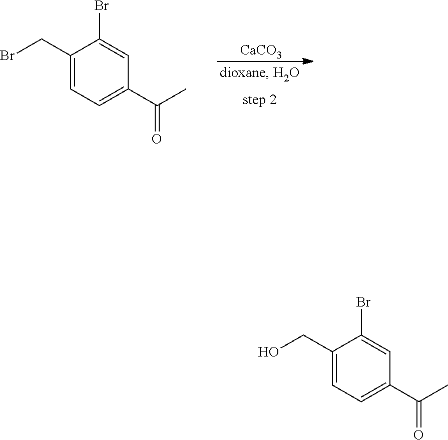

1-[3-bromo-4-(hydroxymethyl)phenyl]ethan-1-one: To a solution of 1-[3-bromo-4-(bromomethyl)phenyl]ethan-1-one (28.95 g, 99.16 mmol, 1.00 equiv.) in dioxane (150 mL) and water (150 mL), CaCO₃ (28.95 g, 289.50 mmol, 3.00 equiv.) was added. The resulting solution was stirred for 16 h at 100° C. The solids were filtered out. The filtrate was extracted with ethyl acetate (2×300 mL) and the organic layers were combined, dried over anhydrous sodium sulfate and concentrated under vacuum. The crude product was purified by SiO₂ chromatography, eluted with EA/PE (1:4) to afford 19.5 g (86%) of 1-[3-bromo-4-(hydroxymethyl) phenyl]ethan-1-one as a white solid.

Step 3

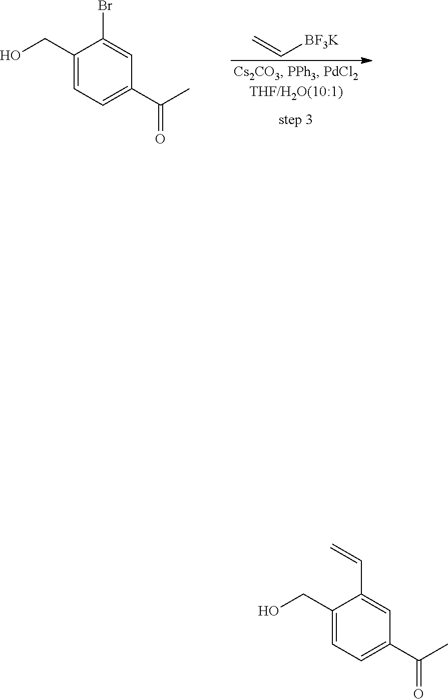

1-[3-ethenyl-4-(hydroxymethyl)phenyl]ethan-1-one: To a solution of 1-[3-bromo-4-(hydroxymethyl)phenyl]ethan-1-one (19.5 g, 85.13 mmol, 1.00 equiv.) in THF/H₂O (10:1) (250 mL), potassium vinyltrifluoroborate (14.9 g, 111.24 mmol, 1.30 equiv.), PdCl₂ (0.3 g, 0.02 equiv), PPh₃ (1.34 g, 5.11 mmol, 0.06 equiv.), Cs₂CO₃ (83.6 g, 256.58 mmol, 3.00 equiv.) was added. The resulting solution was stirred for 24 h at 85° C. The resulting solution was diluted with 300 mL of ethyl acetate. Then the solids were filtered out. The organic layers were washed with brine (2×300 mL), dried over anhydrous sodium sulfate and concentrated under vacuum. The crude product was purified by SiO₂ chromatography, eluted with EA/PE (1:4) to afford 10 g (67%) of 1-[3-ethenyl-4-(hydroxymethyl) phenyl]ethan-1-one as yellow oil. LC-MS: m/z=177 [M+H]⁺.

Step 4

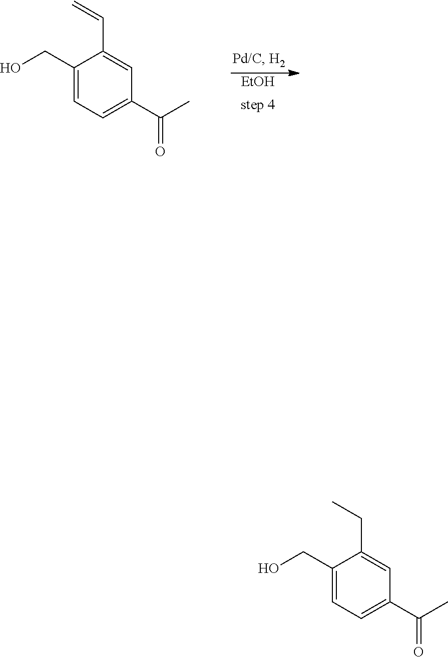

1-[3-ethyl-4-(hydroxymethyl)phenyl]ethan-1-one: To a solution of 1-[3-ethenyl-4-(hydroxymethyl)phenyl]ethan-1-one (5 g, 28.41 mmol, 1.00 equiv.) in ethanol (20 mL), Palladium carbon (1 g) was added. The mixture was purged with H₂. The mixture was stirred for 3 h at 20° C. The reaction was monitored by LCMS. Then the solids were filtered out. The filtrate was concentrated to afford 4.5 g (89%) of 1-[3-ethyl-4-(hydroxymethyl)phenyl]ethan-1-one as white oil. LC-MS: m/z=179 [M+H]⁺.

Step 5

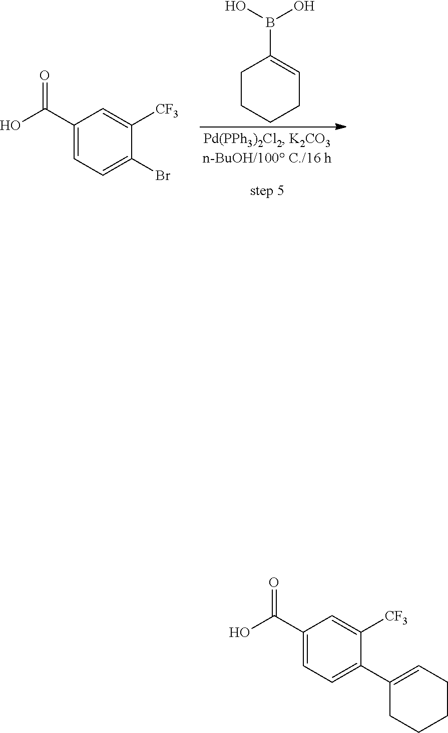 [SMILES: O=C(O)c1ccc(Br)c(C(F)(F)F)c1 ; OB(O)C1=CCCCC1 ; O=C(O)c1ccc(C2=CCCCC2)c(C(F)(F)F)c1]

4-(cyclohex-1-en-1-yl)-3-(trifluoromethyl)benzoic acid: To a solution of 4-bromo-3-(trifluoromethyl)benzoic acid (7.12 g, 26.47 mmol, 1.00 equiv.) in n-BuOH (100 mL), (cyclohex-1-en-1-yl)boronic acid (3.67 g, 29.14 mmol, 1.10 equiv.), Pd(Pph₃)₂Cl₂ (370 mg, 0.53 mmol, 0.02 equiv.), and potassium carbonate (5.48 g, 39.65 mmol, 1.50 equiv.) was added. The resulting solution was stirred overnight at 100° C. The reaction mixture was cooled and quenched by water (200 ml). The resulting solution was extracted with ethyl acetate (3×100 mL) and the organic layers combined, then washed with brine (2×50 mL) and dried over anhydrous sodium sulfate and concentrated under vacuum. The residue was purified by a silica gel column, eluted with ethyl acetate/petroleum ether (1:15). The collected fractions were combined and concentrated under vacuum to afford 6.4 g (89%) of 4-(cyclohex-1-en-1-yl)-3-(trifluoromethyl) benzoic acid as a light brown solid. LC-MS: m/z=269 [M−H]⁻.

Step 6

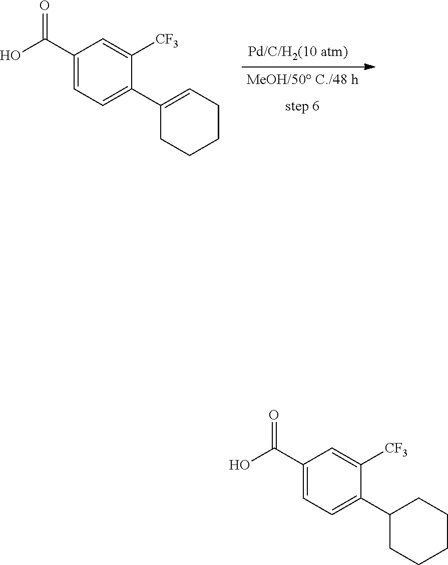 [SMILES: O=C(O)c1ccc(C2=CCCCC2)c(C(F)(F)F)c1 ; O=C(O)c1ccc(C2CCCCC2)c(C(F)(F)F)c1]

4-cyclohexyl-3-(trifluoromethyl)benzoic acid: To a solution of 4-(cyclohex-1-en-1-yl)-3-(trifluoromethyl)benzoic acid (6.4 g, 23.68 mmol, 1.00 equiv.) in methanol (60 mL), Pd/C (0.64 g) was added. The resulting solution was stirred for 72 h at 50° C. with an inert atmosphere of H₂ (10 atm.). The reaction mixture was cooled. The solids were filtered out. The resulting mixture was concentrated under vacuum to afford 5.7 g (88%) of 4-cyclohexyl-3-(trifluoromethyl) benzoic acid as a white solid. LC-MS: m/z=271 [M−H]⁻.

Step 7

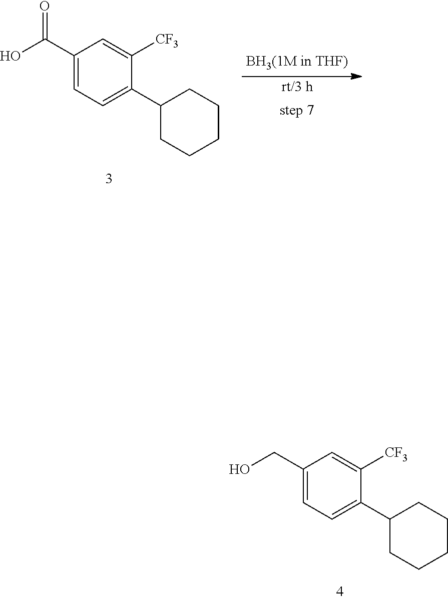 [SMILES: O=C(O)c1ccc(C2CCCCC2)c(C(F)(F)F)c1 ; OCc1ccc(C2CCCCC2)c(C(F)(F)F)c1]

[4-cyclohexyl-3-(trifluoromethyl)phenyl]methanol: To a solution of 4-cyclohexyl-3-(trifluoromethyl) benzoic acid (2 g, 7.35 mmol, 1.00 equiv.) BH₃ (1M in tetrahydrofuran) (15 mL, 2.00 equiv.) was added dropwise with stirring at 0° C. in 30 min. The resulting solution was stirred for 3 h at room temperature and monitored by TLC. The reaction was then quenched by the addition of water slowly and extracted with ethyl acetate (3×50 mL) and the organic layers were combined. The mixture was dried over anhydrous sodium sulfate and concentrated under vacuum to afford 1.85 g (98%) of [4-cyclohexyl-3-(trifluoromethyl)phenyl] methanol as a white solid.

Step 8

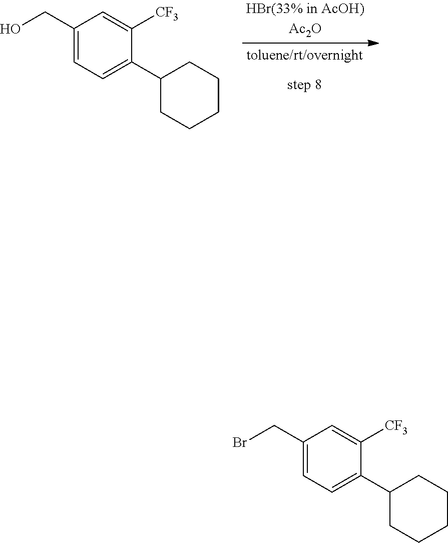 [SMILES: OCc1ccc(C2CCCCC2)c(C(F)(F)F)c1 ; FC(F)(F)c1cc(CBr)ccc1C1CCCCC1]

4-(bromomethyl)-1-cyclohexyl-2-(trifluoromethyl)benzene: To a solution of [4-cyclohexyl-3-(trifluoromethyl)phenyl]methanol (1.85 g, 7.16 mmol, 1.00 equiv.) in toluene (15 mL), HBr (33% in AcOH) (33.3 mL) was added dropwise and then Ac₂O (3.3 mL) dropwise with stirring at 0° C. in 30 min. The resulting solution was stirred overnight at room temperature. The reaction was quenched by the addition of water and extracted with ethyl acetate (3×100 mL). The combined organic layers was washed with saturated sodium bicarbonate (2×50 mL), water (2×50 mL) and brine (2×50 mL), dried over anhydrous sodium sulfate and concentrated under vacuum to afford 1.9 g (83%) of 4-(bromomethyl)-1-cyclohexyl-2-(trifluoromethyl)benzene as light brown oil.

Step 9

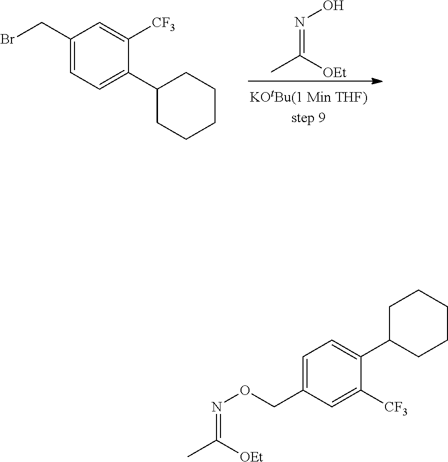

(Z)-(ethyl-N-[[4-cyclohexyl-3-(trifluoromethyl)phenyl]methoxy]ethane carboximidate): To a solution of (Z)-(ethyl N-hydroxyethenecarboximidate) (320 mg, 3.10 mmol, 1.00 equiv.) in tetrahydrofuran (5 mL), KOBu^{t}(1M in tetrahydrofuran) (6.2 mL, 2.00 equiv.) was added dropwise with stirring at 0-5° C. in 5 min. The resulting solution was stirred for 1 h at 0-5° C. To this was added a solution of 4-(bromomethyl)-1-cyclohexyl-2-(trifluoromethyl)benzene (1 g, 3.11 mmol, 1.00 equiv.) in tetrahydrofuran (5 mL) dropwise with stirring. The resulting solution was stirred for 4 h at 0-5° C. The reaction was then quenched by the addition of water and extracted with ethyl acetate (3×100 mL) and the organic layers were combined. The resulting mixture was washed with brine (2×50 mL), dried over anhydrous sodium sulfate and concentrated under vacuum to afford 1 g (94%) of (Z)-(ethyl N-[[4-cyclohexyl-3-(trifluoromethyl)phenyl] methoxy]ethenecarboximidate) as light yellow oil. LC-MS: m/z=344.05 [M+H]⁺.

Step 10

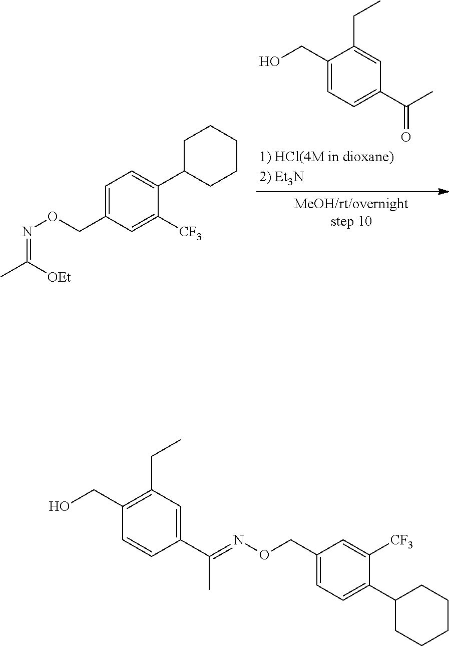 [SMILES: CCO/C(C)=N\OCc1ccc(C2CCCCC2)c(C(F)(F)F)c1 ; CCc1cc(C(C)=O)ccc1CO ; CCc1cc(/C(C)=N/OCc2ccc(C3CCCCC3)c(C(F)(F)F)c2)ccc1CO]

[4-[(1E)-1-([[4-cyclohexyl-3-(trifluoromethyl)phenyl]methoxy]imino)ethyl]-2-ethylphenyl]methanol: To a solution of (Z)-(ethyl N-[[4-cyclohexyl-3-(trifluoromethyl)phenyl]methoxy] ethenecarboximidate) (500 mg, 1.46 mmol, 1.00 equiv.) in methanol (5 mL), hydrogen chloride (4 M in dioxane) (0.73 mL, 0.50 equiv.) was added. The resulting solution was stirred for 30 minutes at room temperature. To this was added triethylamine (221 mg, 2.18 mmol, 1.50 equiv.) to adjust the pH value to 4-6, then the mixture was added a solution of 1-[3-ethyl-4-(hydroxymethyl)phenyl]ethan-1-one (259.5 mg, 1.46 mmol, 1.00 equiv) in methanol (3 mL) dropwise with stirring. The resulting solution was stirred overnight at room temperature. The resulting solution was diluted with water (20 mL) and extracted with ethyl acetate (3×50 mL) and the organic layers were combined. The resulting mixture was washed with brine (50 mL), dried over anhydrous sodium sulfate and concentrated under vacuum. The residue was purified by a silica gel column, eluted with ethyl acetate/petroleum ether (1:5-1:4) to afford 600 mg (95%) of [4-[(1E)-1-([[4-cyclohexyl-3-(trifluoromethyl)phenyl]methoxy]imino) ethyl]-2-ethylphenyl]methanol as light yellow oil. LC-MS: m/z=434.15 [M+H]⁺.

Step 11

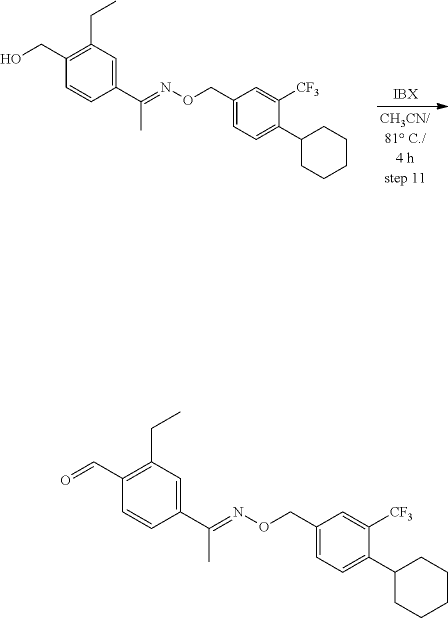 [SMILES: CCc1cc(/C(C)=N/OCc2ccc(C3CCCCC3)c(C(F)(F)F)c2)ccc1CO ; CCc1cc(/C(C)=N/OCc2ccc(C3CCCCC3)c(C(F)(F)F)c2)ccc1C=O]

4-[(1E)-1-([[4-cyclohexyl-3-(trifluoromethyl)phenyl]methoxy]imino)ethyl]-2-ethylbenzaldehyde: To a solution of [4-[(1E)-1-([[4-cyclohexyl-3-(trifluoromethyl)phenyl]methoxy]imino) ethyl]-2-ethylphenyl]methanol (550 mg, 1.27 mmol, 1.00 equiv.) in acetonitrile (10 mL), IBX (711 mg, 2.54 mmol, 2.00 equiv.) was added. The resulting solution was stirred for 4 h at 81° C. The reaction mixture was cooled and concentrated under vacuum then diluted with ethyl acetate (30 mL). The solids were filtered out and concentrated under vacuum to afford 500 mg (91%) of 4-[(1E)-1-([[4-cyclohexyl-3-(trifluoromethyl) phenyl]methoxy]imino)ethyl]-2-ethylbenzaldehyde as an off-white solid. LC-MS: m/z=432.10 [M+H]⁺.

Step 12

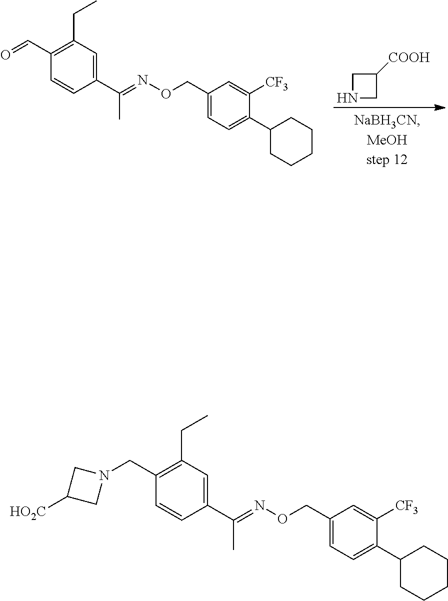 [SMILES: CCc1cc(/C(C)=N/OCc2ccc(C3CCCCC3)c(C(F)(F)F)c2)ccc1C=O ; O=C(O)C1CNC1 ; CCc1cc(/C(C)=N/OCc2ccc(C3CCCCC3)c(C(F)(F)F)c2)ccc1CN1CC(C(=O)O)C1]

1-([4-[(1E)-1-([[4-cyclohexyl-3-(trifluoromethyl)phenyl]methoxy]imino)ethyl]-2-ethylphenyl]methyl)azetidine-3-carboxylic acid: To a solution of 4-[(1E)-1-([[4-cyclohexyl-3-(trifluoromethyl) phenyl]methoxy]imino) ethyl]-2-ethylbenzaldehyde (500 mg, 1.16 mmol, 1.00 equiv.) in methanol (10 mL), azetidine-3-carboxylic acid (234.3 mg, 2.32 mmol, 2.00 equiv.) and acetic acid (0.6 mL) was added. The resulting solution was stirred for 20 min at room temperature. Then NaBH₃CN (36.41 mg, 0.58 mmol, 0.50 equiv) was added in portions. The resulting solution was stirred overnight at room temperature. The resulting solution was diluted with water (10 mL). The mixture was extracted with ethyl acetate (3×30 mL), dried over anhydrous sodium sulfate and concentrated under vacuum. The crude product was purified by Prep-HPLC with the following conditions: Column, XBridge Shield RP18 OBD Column, 5 um, 19*150 mm; mobile phase, Water (0.1% FA) and ACN (35.0% ACN up to 55.0% in 7 min); Detector, UV 254 nm to afford 120 mg (20%) of 1-([4-[(1E)-1-([[4-cyclohexyl-3-(trifluoromethyl)phenyl]methoxy]imino)ethyl]-2-ethylphenyl]methyl)azetidine-3-carboxylic acid as a white solid. ¹H NMR (300 MHz, CD₃OD) δ: 7.75-7.48 (m, 5H), 7.40-7.38 (m, 1H), 5.21 (s, 2H), 4.44 (s, 2H), 4.22-4.19 (m, 4H), 3.46-3.41 (m, 1H), 2.99-2.88 (m, 1H), 2.78 (q, J=7.5 Hz, 2H), 2.24 (s, 3H), 1.92-1.71 (m, 5H), 1.64-1.30 (m, 5H), 1.23 (t, J=7.5 Hz, 3H). LC-MS: m/z=517 [M+H]⁺.

### Example 2: 1-([4-[(1E)-1-([[4-cyclohexyl-3-(trifluoromethyl)phenyl]methoxy]imino)ethyl]-2-ethylphenyl](²H₂)methyl)azetidine-3-carboxylic acid

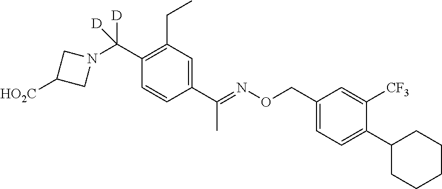 [SMILES: [2H]C([2H])(c1ccc(/C(C)=N/OCc2ccc(C3CCCCC3)c(C(F)(F)F)c2)cc1CC)N1CC(C(=O)O)C1]

Step 1

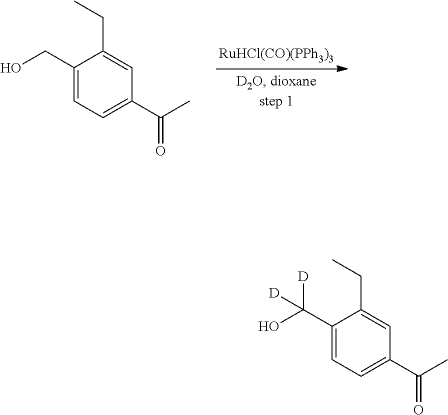

1-[3-ethyl-4-[hydroxy(²H₃)methyl]phenyl]ethan-1-one: To a solution of 1-[3-ethyl-4-(hydroxymethyl)phenyl]ethan-1-one (1.2 g, 6.73 mmol, 1.00 equiv.) in D₂O (1.83 mL, 13.56 equiv) and dioxane (12 mL), RuHCl(CO)(PPh₃)₃ (193 mg, 0.20 mmol, 0.03 equiv.) was added. The resulting solution was stirred for 4 h at 100° C. The reaction mixture was extracted with ethyl acetate (2×20 mL). The organic layers were washed with brine (2×30 mL), dried over anhydrous sodium sulfate and concentrated under vacuum. The procedure was repeated three times. Then the crude product was purified by SiO₂ chromatography, eluted with EA/PE (1:4) to afford 900 mg (74%) of 1-[3-ethyl-4-[hydroxy(²H₃)methyl]phenyl] ethan-1-one as light yellow oil. LC-MS: m/z=181 [M+H]⁺.

Step 2

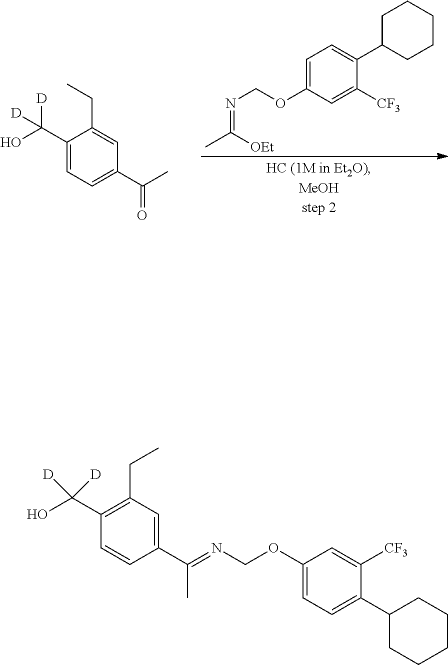 [SMILES: [2H]C([2H])(O)c1ccc(C(C)=O)cc1CC ; CCO/C(C)=N\COc1ccc(C2CCCCC2)c(C(F)(F)F)c1 ; [2H]C([2H])(O)c1ccc(/C(C)=N/COc2ccc(C3CCCCC3)c(C(F)(F)F)c2)cc1CC]

[4-[(1E)-1-([[4-cyclohexyl-3-(trifluoromethyl)phenyl](²H₂)methoxy]imino)ethyl]-2-ethylphenyl]methanol: To a solution of (Z)-(ethyl N-[[4-cyclohexyl-3-(trifluoromethyl)phenyl]methoxy] ethenecarboximidate) (800 mg, 2.33 mmol, 1.00 equiv.) in methanol (10 mL), hydrogen chloride (4 M in dioxane) (1.2 mL, 0.50 equiv) was added. The resulting solution was stirred for 30 minutes at room temperature. To this was added triethylamine (353 mg, 3.49 mmol, 1.50 equiv.) to adjust the pH value to 4-6. Then 1-[3-ethyl-4-[hydroxy (²H₂)methyl]phenyl]ethan-1-one (419.8 mg, 2.33 mmol, 1.00 equiv.) was added and the resulting solution was stirred overnight at room temperature. The resulting solution was diluted with 30 mL of water and extracted with ethyl acetate (3×50 mL). The combined organic layers was washed with brine (50 mL), dried over anhydrous sodium sulfate and concentrated under vacuum. The residue was purified by a silica gel column, eluted with ethyl acetate/petroleum ether (1:5-1:4) to afford 800 mg (79%) of [4-[(1E)-1-([[4-cyclohexyl-3-(trifluoromethyl)phenyl](²H₂)methoxy] imino)ethyl]-2-ethylphenyl]methanol as brown oil. LC-MS: m/z=436.15[M+H]⁺.

Step 3

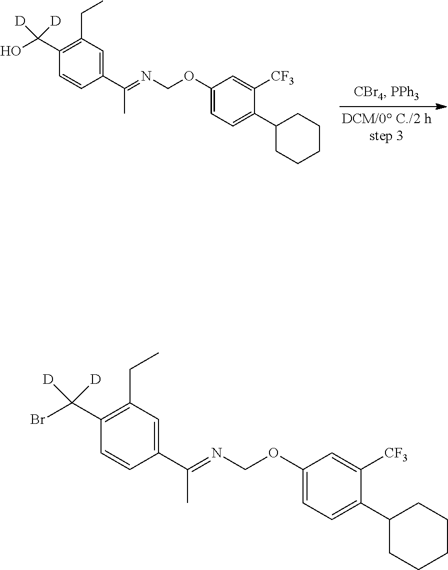 [SMILES: [2H]C([2H])(O)c1ccc(/C(C)=N/COc2ccc(C3CCCCC3)c(C(F)(F)F)c2)cc1CC ; [2H]C([2H])(Br)c1ccc(/C(C)=N/COc2ccc(C3CCCCC3)c(C(F)(F)F)c2)cc1CC]

(E)-(1-[4-[bromo(²H₂)methyl]-3-ethylphenyl]ethylidene)([[4-cyclohexyl-3-(trifluoromethyl)phenyl]methoxy])amine: To a solution of [4-[(1E)-1-([[4-cyclohexyl-3-(trifluoromethyl)phenyl]methoxy] imino)ethyl]-2-ethylphenyl](²H₂)methanol (800 mg, 1.84 mmol, 1.00 equiv.) in dichloromethane (10 mL), CBr₄ (915.2 mg, 2.76 mmol, 1.50 equiv.) and PPh₃ (724.4 mg, 2.76 mmol, 1.50 equiv.) were added. The resulting solution was stirred for 2 h at 0° C. The resulting solution was diluted with water (20 mL) and extracted with ethyl acetate (3×30 mL). The combined organic layers was washed with brine (20 mL), dried over anhydrous sodium sulfate and concentrated under vacuum. The residue was purified by a silica gel column, eluted with ethyl acetate/petroleum ether (1:50) to afford 800 mg (87%) of (E)-(1-[4-[bromo(²H₂)methyl]-3-ethylphenyl]ethylidene)([[4-cyclohexyl-3-(trifluoromethyl)phenyl]methoxy])amine as brown oil. LC-MS: m/z=498[M+H]⁺.

Step 4

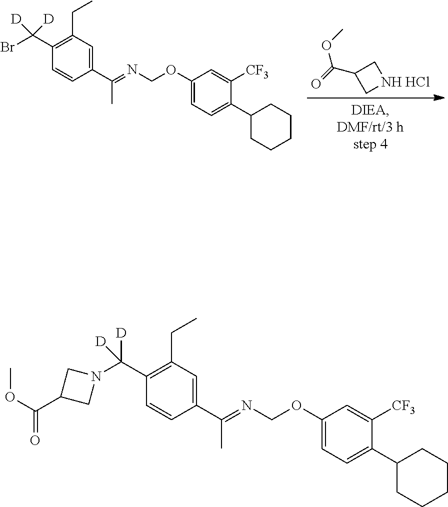

methyl1-([4-[(1E)-1-([[4-cyclohexyl-3-(trifluoromethyl)phenyl]methoxy]imino) ethyl]-2-ethylphenyl](²H₂)methyl)azetidine-3-carboxylate: To a solution of (E)-(1-[4-[bromo(²H₂)methyl]-3-ethylphenyl]ethylidene)([[4-cyclohexyl-3-(trifluoromethyl)phenyl]methoxy])amine (800 mg, 1.61 mmol, 1.00 equiv.) in DMF (10 mL), DIEA (621.2 mg, 4.82 mmol, 3.00 equiv.) and methyl azetidine-3-carboxylate hydrochloride (365 mg, 2.41 mmol, 1.50 equiv.) were added. The resulting solution was stirred for 3 h at room temperature. The resulting solution was diluted with water (20 mL) and extracted with ethyl acetate (3×30 mL). The combined organic layers was washed with brine (20 mL), dried over anhydrous sodium sulfate and concentrated under vacuum. The residue was purified by a silica gel column, eluted with ethyl acetate/petroleum ether (1:9) to afford 700 mg (82%) of methyl 1-([4-[(1E)-1-([[4-cyclohexyl-3-(trifluoromethyl)phenyl]methoxy]imino)ethyl]-2-ethylphenyl](²H₂)methyl)azetidine-3-carboxylate as light brown oil. LC-MS: m/z=533.25[M+H]⁺.

Step 5

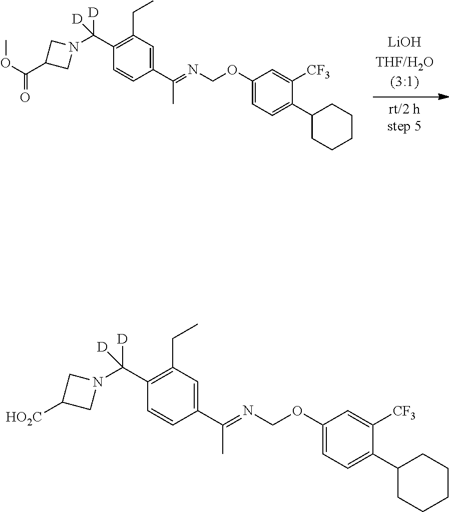 [SMILES: [2H]C([2H])(c1ccc(/C(C)=N/COc2ccc(C3CCCCC3)c(C(F)(F)F)c2)cc1CC)N1CC(C(=O)OC)C1 ; [2H]C([2H])(c1ccc(/C(C)=N/COc2ccc(C3CCCCC3)c(C(F)(F)F)c2)cc1CC)N1CC(C(=O)O)C1]

1-([4-[(1E)-1-([[4-cyclohexyl-3-(trifluoromethyl)phenyl]methoxy]imino)ethyl]-2-ethylphenyl](²H₂)methyl)azetidine-3-carboxylic acid: To a solution of methyl 1-([4-[(1E)-1-([[4-cyclohexyl-3-(trifluoromethyl)phenyl]methoxy]imino)ethyl]-2-ethylphenyl](²H₂)methyl)azetidine-3-carboxylate (500 mg, 0.94 mmol, 1.00 equiv) in tetrahydrofuran:H₂O (3:1) (12 mL), LiOH (67.45 mg, 2.82 mmol, 3.00 equiv.) was added. The resulting solution was stirred for 3 h at room temperature. Then the pH value of the mixture was adjusted to 3-5 with hydrochloric acid (1 M). The mixture was extracted with ethyl acetate (3×30 mL), dried over anhydrous sodium sulfate and concentrated under vacuum. The crude product was purified by Prep-HPLC with the following conditions: Column, XBridge Shield RP18 OBD Column, Sum, 19*150 mm; mobile phase, Water (0.1% FA) and ACN (35.0% ACN up to 55.0% in 7 min); Detector, UV 254 nm to afford 120 mg (25%) of 1-([4-[(1E)-1-([[4-cyclohexyl-3-(trifluoromethyl) phenyl] methoxy] imino)ethyl]-2-ethylphenyl](²H₂)methyl)azetidine-3-carboxylic acid as a white solid. ¹H NMR (300 MHz, CD₃OD) δ: 7.66-7.51 (m, 5H), 7.41-7.38 (m, 1H), 5.22 (s, 2H), 4.24-4.22 (m, 4H), 3.49-3.41 (m, 1H), 2.99-2.90 (m, 1H), 2.89 (q, J=7.5 Hz, 2H), 2.25 (s, 3H), 1.87-1.75 (m, 5H), 1.58-1.36 (m, 5H), 1.23 (t, J=7.5 Hz, 3H). LC-MS: m/z=519.35 [M+H]⁺.

### Example 3: 1-([4-[(1E)-1-([[4-cyclohexyl-3-(trifluoromethyl)phenyl]methoxy]imino)ethyl]-2-²H₂)ethylphenyl](²H₅)methyl)azetidine-3-carboxylic acid

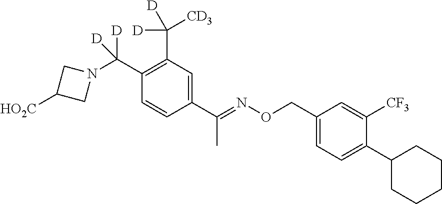 [SMILES: [2H]C([2H])(c1ccc(/C(C)=N/OCc2ccc(C3CCCCC3)c(C(F)(F)F)c2)cc1C([2H])([2H])C([2H])([2H])[2H])N1CC(C(=O)O)C1]

Step 1

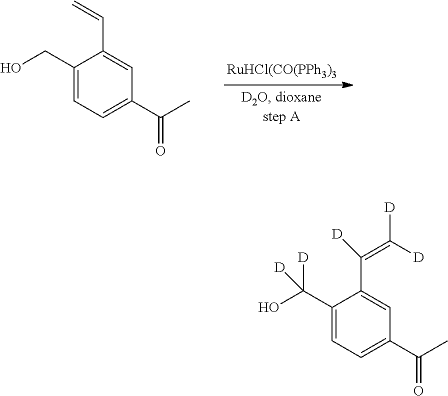 [SMILES: C=Cc1cc(C(C)=O)ccc1CO ; [2H]C([2H])=C([2H])c1cc(C(C)=O)ccc1C([2H])([2H])O]

1-[3-(²H₃)ethenyl-4-[hydroxy(²H₂)methyl]phenyl]ethan-1-one: To a solution of 1-[3-ethenyl-4-(hydroxymethyl)phenyl]ethan-1-one (3 g, 17.02 mmol, 1.00 equiv.) in D₂O (4.62 g, 13.56 equiv.) and dioxane (30 mL), RuHCl(CO)(PPh₃)₃ (487 mg, 0.51 mmol, 0.03 equiv.) was added. The resulting solution was stirred for 4 h at 100° C. The reaction mixture was extracted with ethyl acetate (2×30 mL). The organic layers were washed with brine (2×30 mL), dried over anhydrous sodium sulfate and concentrated under vacuum. The procedure was repeated three times. Then the crude product was purified by SiO₂ chromatography, eluted with EA/PE (1:4) to afford 2.5 g (81%) of 1-[3-(²H₃)ethenyl-4-[hydroxy(²H₂)methyl] phenyl]ethan-1-one as light yellow oil. LC-MS: m/z=182 [M+H]⁺.

Step 2

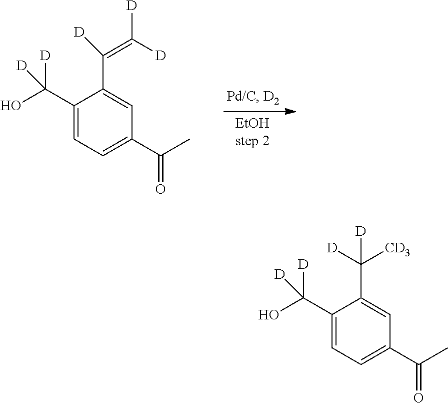 [SMILES: [2H]C([2H])=C([2H])c1cc(C(C)=O)ccc1C([2H])([2H])O ; [2H]C([2H])(O)c1ccc(C(C)=O)cc1C([2H])([2H])C([2H])([2H])[2H]]

1-[3-(²H₅)ethyl-4-[hydroxy(²H₂)methyl]phenyl]ethan-1-one: To a solution of 1-[3-(²H₃)ethenyl-4-[hydroxy(²H₂)methyl]phenyl]ethan-1-one (1.8 g, 9.93 mmol, 1.00 equiv.) in ethanol (20 mL), Palladium carbon (360 mg) was added. The mixture was purged with D₂. The mixture was stirred for 3 h at 20° C. The reaction was monitored by LCMS. Then the solids were filtered out. The filtrate was concentrated to afford 1.8 g (98%) of 1-[3-(²H₅)ethyl-4-[hydroxy(²H₂)methyl] phenyl]ethan-1-one as yellow oil. LC-MS: m/z=186 [M+H]⁺.

Step 3

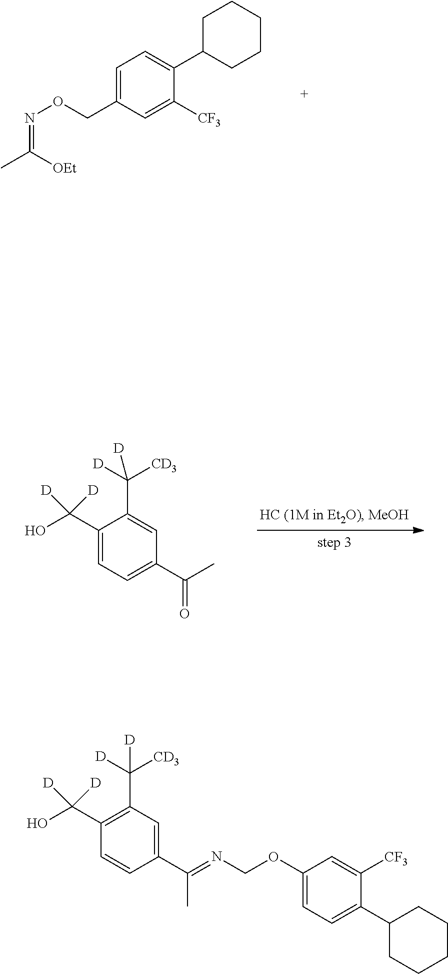 [SMILES: CCO/C(C)=N\OCc1ccc(C2CCCCC2)c(C(F)(F)F)c1 ; [2H]C([2H])(O)c1ccc(C(C)=O)cc1C([2H])([2H])C([2H])([2H])[2H] ; [2H]C([2H])(O)c1ccc(/C(C)=N/COc2ccc(C3CCCCC3)c(C(F)(F)F)c2)cc1C([2H])([2H])C([2H])([2H])[2H]]

[4-[(1E)-1-([[4-cyclohexyl-3-(trifluoromethyl)phenyl]methoxy]imino)ethyl]-2-(²H₅)ethylphenyl](²H₂)methanol: To a solution of (Z)-(ethyl N-[[4-cyclohexyl-3-(trifluoromethyl)phenyl]methoxy] ethenecarboximidate) (800 mg, 2.33 mmol, 1.00 equiv.) in methanol (10 mL), hydrogen chloride (4 M in dioxane) (1.2 mL, 0.50 equiv.) was added. The resulting solution was stirred for 30 minutes at room temperature. To this, triethylamine (353 mg, 3.49 mmol, 1.50 equiv) was added to adjust the pH value to 4-6, then 1-[3-(²H₅)ethyl-4-[hydroxy(²H₂)methyl]phenyl]ethan-1-one (431.5 mg, 2.33 mmol, 1.00 equiv.) was added. The resulting solution was stirred overnight at room temperature. The resulting solution was diluted with 30 mL of water, extracted with ethyl acetate (3×50 mL) and the organic layers were combined, washed with brine (50 mL), dried over anhydrous sodium sulfate and concentrated under vacuum. The residue was purified by a silica gel column, eluted with ethyl acetate/petroleum ether (1:5-1:4) to afford 800 mg (78%) of [4-[(1E)-1-([[4-cyclohexyl-3-(trifluoromethyl)phenyl]methoxy]imino)ethyl]-2-(²H₅)ethylphenyl] (²H₂) methanol as brown oil. LC-MS: m/z=441.15[M+H]⁺.

Step 4

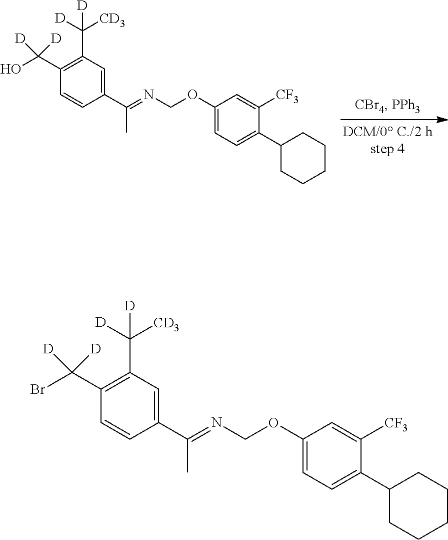 [SMILES: [2H]C([2H])(O)c1ccc(/C(C)=N/COc2ccc(C3CCCCC3)c(C(F)(F)F)c2)cc1C([2H])([2H])C([2H])([2H])[2H] ; [2H]C([2H])(Br)c1ccc(/C(C)=N/COc2ccc(C3CCCCC3)c(C(F)(F)F)c2)cc1C([2H])([2H])C([2H])([2H])[2H]]

(E)-(1-[4-[bromo(²H₂)methyl]-3-(²H₅)ethylphenyl]ethylidene)([[4-cyclohexyl-3-(trifluoromethyl)phenyl]methoxy]) amine: To a solution of [4-[(1E)-1-([[4-cyclohexyl-3-(trifluoromethyl)phenyl]methoxy]imino)ethyl]-2-(²H₅)ethylphenyl](²H₂)methanol (800 mg, 1.82 mmol, 1.00 equiv) in dichloromethane (10 mL), CBr₄ (903 mg, 1.50 equiv) and PPh₃ (714.5 mg, 2.72 mmol, 1.50 equiv.) was added with stirring for 2 h at 0° C. The resulting solution was diluted with water (20 mL), extracted with ethyl acetate (3×30 mL). The combined organic layers was washed with brine (20 mL), dried over anhydrous sodium sulfate and concentrated under vacuum. The residue was purified by a silica gel column, eluted with ethyl acetate/petroleum ether (1:50) to afford 800 mg (88%) of (E)-(1-[4-[bromo(²H₂)methyl]-3-(²H₅)ethylphenyl]ethylidene)([[4-cyclohexyl-3-(trifluoromethyl) phenyl]methoxy]) amine as light brown oil. LC-MS: m/z=503.10[M+H]⁺.

Step 5

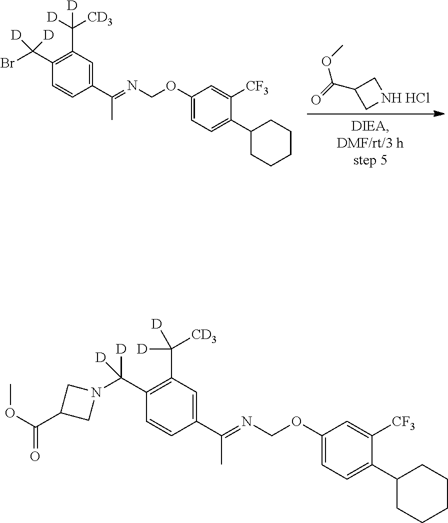

Methyl 1-([4-[(1E)-1-([[4-cyclohexyl-3-(trifluoromethyl)phenyl]methoxy]imino) ethyl]-2-(²H₅)ethylphenyl] (²H₂)methyl)azetidine-3-carboxylate: To a solution of (E)-(1-[4-[bromo(²H₂)methyl]-3-(²H₅)ethylphenyl]ethylidene)([[4-cyclohexyl-3-(trifluoromethyl)phenyl]methoxy])amine (800 mg, 1.59 mmol, 1.00 equiv.) in N,N-dimethylformamide (10 mL), DIEA (615.5 mg, 4.77 mmol, 3.00 equiv.) and methyl azetidine-3-carboxylate hydrochloride (361.6 mg, 2.39 mmol, 1.50 equiv.) were added. The resulting solution was stirred for 3 h at room temperature. The resulting solution was diluted with water (20 mL), extracted with ethyl acetate (3×30 mL) and the organic layers were combined, washed with brine (20 mL), dried over anhydrous sodium sulfate and concentrated under vacuum. The residue was purified by a silica gel column, eluted with ethyl acetate/petroleum ether (1:9) to afford 700 mg (82%) of methyl 1-([4-[(1E)-1-([[4-cyclohexyl-3-(trifluoromethyl)phenyl]methoxy]imino)ethyl]-2-(²H₅)ethylphenyl] (²H₂)methyl)azetidine-3-carboxylate as light brown oil. LC-MS: m/z=538.30[M+H]⁺.

Step 6

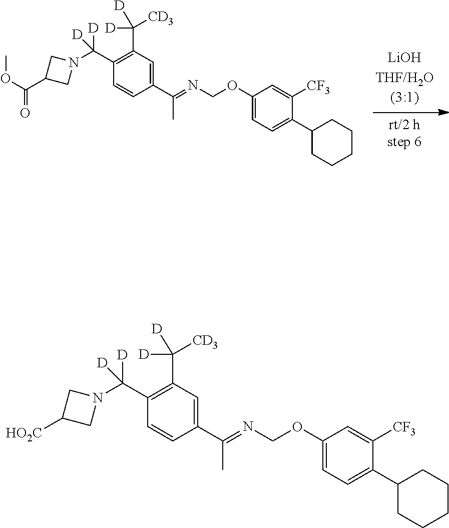 [SMILES: [2H]C([2H])(c1ccc(/C(C)=N/COc2ccc(C3CCCCC3)c(C(F)(F)F)c2)cc1C([2H])([2H])C([2H])([2H])[2H])N1CC(C(=O)OC)C1 ; [2H]C([2H])(c1ccc(/C(C)=N/COc2ccc(C3CCCCC3)c(C(F)(F)F)c2)cc1C([2H])([2H])C([2H])([2H])[2H])N1CC(C(=O)O)C1]

1-([4-[(1E)-1-([[4-cyclohexyl-3-(trifluoromethyl)phenyl]methoxy]imino)ethyl]-2-(²H₂)ethylphenyl](²H₅)methyl)azetidine-3-carboxylic acid: To a solution of methyl 1-([4-[(1E)-1-([[4-cyclohexyl-3-(trifluoromethyl)phenyl] methoxy]imino)ethyl]-2-(²H₅)ethylphenyl](²H₂)methyl)azetidine-3-carboxylate (500 mg, 0.93 mmol, 1.00 equiv.) in tetrahydrofuran: H₂O (3:1) (12 mL), LiOH (66.81 mg, 2.79 mmol, 3.00 equiv.) was added. The resulting solution was stirred for 3 h at room temperature. Then the pH value of the mixture was adjusted to 3-5 with hydrochloric acid (1 M). The mixture was extracted with ethyl acetate (3×30 mL), dried over anhydrous sodium sulfate and concentrated under vacuum. The crude product was purified by Prep-HPLC with the following conditions: Column, XBridge Shield RP18 OBD Column, Sum, 19*150 mm; mobile phase, Water (0.1% FA) and ACN (35.0% ACN up to 55.0% in 7 min); Detector, UV 254 nm to afford 120 mg (25%) of 1-([4-[(1E)-1-([[4-cyclohexyl-3-(trifluoromethyl)phenyl]methoxy]imino)ethyl]-2-(²H₂)ethylphenyl] (²H₅) methyl)azetidine-3-carboxylic acid as a white solid. ¹H NMR (300 MHz, CD₃OD) δ: 7.66-7.51 (m, 5H), 7.41-7.33 (m, 1H), 5.21 (s, 2H), 4.23-4.20 (m, 4H), 3.47-3.41 (m, 1H), 2.96-2.87 (m, 1H), 2.24 (s, 3H), 1.87-1.75 (m, 5H), 1.58-1.31 (m, 5H). LC-MS: m/z=524.35 [M+H]⁺.

### Example 4: 1-([4-[1-([[4-cyclohexyl-3-(trifluoromethyl)phenyl]methoxy]imino)(²H₃)ethyl]-2-ethylphenyl]methyl)azetidine-3-carboxylic acid

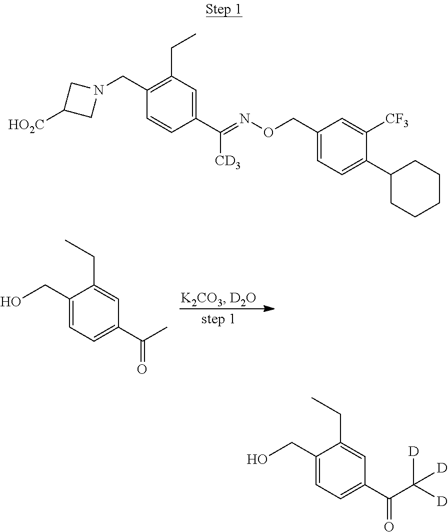 [SMILES: [2H]C([2H])([2H])/C(=N\OCc1ccc(C2CCCCC2)c(C(F)(F)F)c1)c1ccc(CN2CC(C(=O)O)C2)c(CC)c1 ; CCc1cc(C(C)=O)ccc1CO ; [2H]C([2H])([2H])C(=O)c1ccc(CO)c(CC)c1]

1-[3-ethyl-4-(hydroxymethyl)phenyl](²H₃)ethan-1-one: To a solution of 1-[3-ethyl-4-(hydroxymethyl)phenyl]ethan-1-one (600 mg, 3.37 mmol, 1.00 equiv.) in D₂O (5 mL), potassium carbonate (1.4 g, 10.13 mmol, 3.00 equiv.) was added. The resulting solution was stirred for 15 h at 100° C. The resulting solution was extracted with 2×10 mL of ethyl acetate and the organic layers were combined, dried over anhydrous sodium sulfate and concentrated under vacuum to afford 550 mg (90%) of 1-[3-ethyl-4-(hydroxymethyl)phenyl](²H₃)ethan-1-one as yellow oil. LC-MS: m/z=182 [M+H]⁺.

Step 2

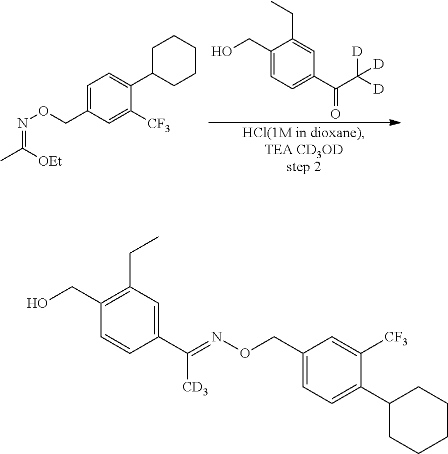

[4-[(1E)-1-([[4-cyclohexyl-3-(trifluoromethyl)phenyl]methoxy]imino)(²H₃)ethyl]-2-ethylphenyl]methanol: To a solution of (Z)-(ethyl N-[[4-cyclohexyl-3-(trifluoromethyl)phenyl]methoxy] ethenecarboximidate) (700 mg, 2.04 mmol, 1.00 equiv.) in CD₃OD (10 mL), hydrogen chloride(4M in dioxane) (1.02 mL) was added. The resulting solution was stirred for 30 min at room temperature. To this solution was added TEA (309 mg, 3.05 mmol, 1.50 equiv.) to adjust the pH value to 4-6. Then a solution of 1-[3-ethyl-4-(hydroxymethyl)phenyl](²H₃)ethan-1-one (369 mg, 2.04 mmol, 1.00 equiv.) in CD₃OD (2 mL) was added dropwise. The resulting solution was stirred overnight at room temperature. Then the resulting solution was concentrated under vacuum to remove CD₃OD. The residue was purified by SiO₂ chromatography, eluted with ethyl acetate/petroleum ether (1:7) to afford 800 mg (90%) of [4-[(1E)-1-([[4-cyclohexyl-3-(trifluoromethyl)phenyl]methoxy]imino)(²H₃)ethyl]-2-ethylphenyl]methanol as light yellow oil. LC-MS: m/z=437 [M+H]⁺.

Step 3

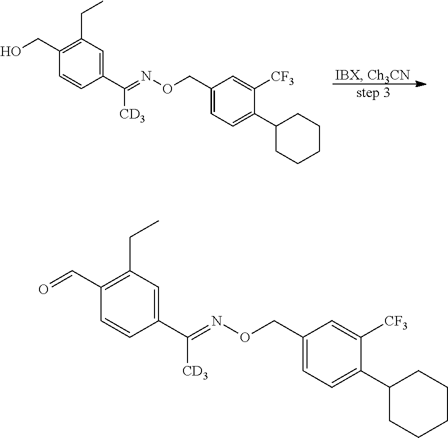 [SMILES: [2H]C([2H])([2H])/C(=N\OCc1ccc(C2CCCCC2)c(C(F)(F)F)c1)c1ccc(CO)c(CC)c1 ; [2H]C([2H])([2H])/C(=N\OCc1ccc(C2CCCCC2)c(C(F)(F)F)c1)c1ccc(C=O)c(CC)c1]

4-[(1E)-1-([[4-cyclohexyl-3-(trifluoromethyl)phenyl]methoxy]imino)(²H₃)ethyl]-2-ethylbenzaldehyde: To a solution of [4-[(1E)-1-([[4-cyclohexyl-3-(trifluoromethyl)phenyl]methoxy]imino)(²H₃)ethyl]-2-ethylphenyl]methanol (650 mg, 1.49 mmol, 1.00 equiv.) in CH₃CN (10 mL), IBX (835 mg, 2.98 mmol, 2.00 equiv.) was added. The resulting solution was stirred for 4 h at 81° C. The reaction mixture was cooled and concentrated under vacuum. Then the mixture was diluted with ethyl acetate (30 mL). The solids were filtered out. The filtrate was concentrated under vacuum to afford 640 mg (99%) of 4-[(1E)-1-([[4-cyclohexyl-3-(trifluoromethyl)phenyl]methoxy]imino)(²H₃)ethyl]-2-ethylbenzaldehyde as yellow oil. LC-MS: m/z=435 [M+H]⁺.

Step 4

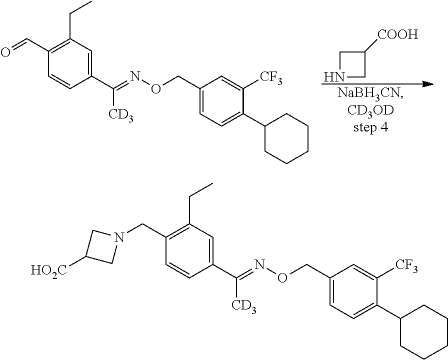 [SMILES: [2H]C([2H])([2H])/C(=N\OCc1ccc(C2CCCCC2)c(C(F)(F)F)c1)c1ccc(C=O)c(CC)c1 ; O=C(O)C1CNC1 ; [2H]C([2H])([2H])/C(=N\OCc1ccc(C2CCCCC2)c(C(F)(F)F)c1)c1ccc(CN2CC(C(=O)O)C2)c(CC)c1]

1-([4-[1-([[4-cyclohexyl-3-(trifluoromethyl)phenyl]methoxy]imino)(²H₃)ethyl]-2-ethylphenyl]methyl)azetidine-3-carboxylic acid: To a solution of 4-[1-([[4-cyclohexyl-3-(trifluoromethyl)phenyl]methoxy]imino)(²H₃) ethyl]-2-ethylbenzaldehyde (600 mg, 1.38 mmol, 1.00 equiv.) in CD₃OD (10 mL), azetidine-3-carboxylic acid (279 mg, 2.76 mmol, 2.00 equiv.) and acetic acid (0.7 mL) was added. The resulting solution was stirred for 20 min at room temperature. Then NaBH₃CN (44 mg, 0.70 mmol, 0.50 equiv.) was added in portions. The resulting solution was stirred overnight at room temperature. The resulting solution was diluted with water (10 mL). The mixture was extracted with ethyl acetate (3×30 mL), dried over anhydrous sodium sulfate and concentrated under vacuum. The crude product was purified by Prep-HPLC with the following conditions: Column, XBridge Shield RP18 OBD Column, Sum, 19*150 mm; mobile phase, Water (0.1% FA) and ACN (35.0% ACN up to 55.0% in 7 min); Detector, UV 254 nm to afford 150 mg (21%) of 1-([4-[1-([[4-cyclohexyl-3-(trifluoromethyl)phenyl]methoxy]imino)(²H₃)ethyl]-2-ethylphenyl]methyl)azetidine-3-carboxylic acid as a white solid. ¹H NMR (300 MHz, CD₃OD) δ: 7.67-7.51 (m, 5H), 7.40-7.37 (m, 1H), 5.22 (s, 2H), 4.43 (s, 2H), 4.21-4.19 (m, 4H), 3.43-3.38 (m, 1H), 2.96-2.82 (m, 1H), 2.79 (q, J=7.5 Hz, 2H), 1.87-1.76 (m, 5H), 1.58-1.32 (m, 5H), 1.23 (t, J=7.5 Hz, 3H). LC-MS: m/z=520 [M+H]⁺.

### Example 5: 1-([4-[(1E)-1-([[4-cyclohexyl-3-(trifluoromethyl)phenyl](²H₂)methoxy]imino) ethyl]-2-ethylphenyl]methyl)azetidine-3-carboxylic acid

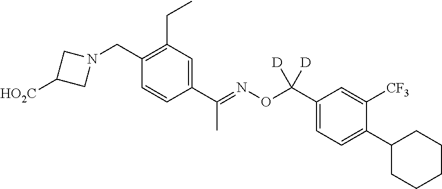 [SMILES: [2H]C([2H])(O/N=C(\C)c1ccc(CN2CC(C(=O)O)C2)c(CC)c1)c1ccc(C2CCCCC2)c(C(F)(F)F)c1]

Step 1

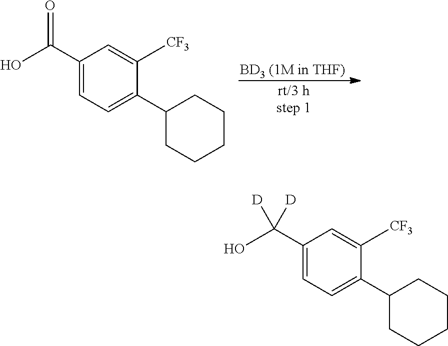 [SMILES: O=C(O)c1ccc(C2CCCCC2)c(C(F)(F)F)c1 ; [2H]C([2H])(O)c1ccc(C2CCCCC2)c(C(F)(F)F)c1]

[4-Cyclohexyl-3-(trifluoromethyl)phenyl](²H₂)methanol: To a solution of 4-cyclohexyl-3-(trifluoromethyl)benzoic acid (2 g, 7.35 mmol, 1.00 equiv.), BD₃ (1M in tetrahydrofuran) (15 mL, 2.00 equiv.) was added dropwise with stirring at 0° C. in 30 min. The resulting solution was stirred for 3 h at room temperature. The reaction was then quenched by the addition of D₂O slowly and extracted with ethyl acetate (3×50 mL). The combined organic layers was dried over anhydrous sodium sulfate and concentrated under vacuum to afford 1.85 g (97%) of [4-cyclohexyl-3-(trifluoromethyl)phenyl](²H₂)methanol as a white solid.

Step 2

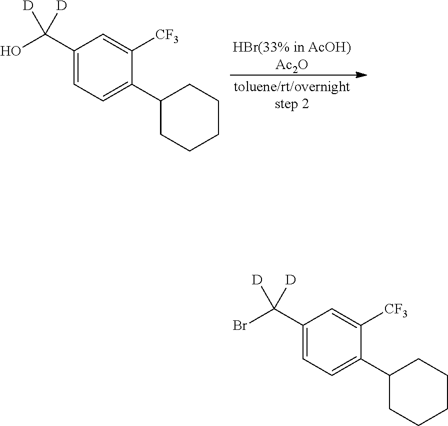 [SMILES: [2H]C([2H])(O)c1ccc(C2CCCCC2)c(C(F)(F)F)c1 ; [2H]C([2H])(Br)c1ccc(C2CCCCC2)c(C(F)(F)F)c1]

4-[Bromo(²H₂)methyl]-1-cyclohexyl-2-(trifluoromethyl)benzene: To a solution of [4-cyclohexyl-3-(trifluoromethyl)phenyl](²H₂)methanol (1.85 g, 7.11 mmol, 1.00 equiv.) in toluene (15 mL), Ac₂O (3.3 mL) was added dropwise with stirring at 0° C. in 30 min. The resulting solution was stirred overnight at room temperature. The reaction was then quenched by the addition of water and extracted with ethyl acetate (3×100 mL) and the organic layers combined then washed with saturated sodium bicarbonate (2×50 mL), water (2×50 mL), brine (2×50 mL), dried over anhydrous sodium sulfate and concentrated under vacuum to afford 1.9 g (83%) of 4-[bromo(²H₂)methyl]-1-cyclohexyl-2-(trifluoromethyl)benzene as light brown oil.

Step 3

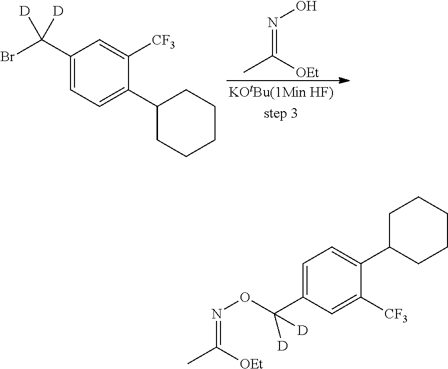

(Z)-(ethylN-[[4-cyclohexyl-3-(trifluoromethyl)phenyl](²H₂)methoxy] ethenecarboximidate): To a solution of (Z)-(ethyl N-hydroxyethenecarboximidate) (610 mg, 5.92 mmol, 1.00 equiv.) in tetrahydrofuran (15 mL), KO^{t}Bu (1M in tetrahydrofuran) (11.9 mL, 2.00 equiv.) was added dropwise with stirring at 0-5° C. in 10 min. The resulting solution was stirred for 1 h at 0-5° C. To this was added a solution of 4-[bromo(²H₂)methyl]-1-cyclohexyl-2-(trifluoromethyl)benzene (1.9 g, 5.88 mmol, 1.00 equiv.) in tetrahydrofuran (5 mL) dropwise with stirring. The resulting solution was stirred for 4 h at 0-5° C. The reaction was then quenched by the addition of water. The resulting solution was extracted with ethyl acetate (3×100 mL). The combined organic layers was washed with brine (2×50 mL), dried over anhydrous sodium sulfate and concentrated under vacuum to afford 1.9 g (93%) of (Z)-(ethyl-N-[[4-cyclohexyl-3-(trifluoromethyl)phenyl](²H₂)methoxy]ethenecarboximidate) as light brown oil. LC-MS: m/z=346.05[M+H]⁺.

Step 4

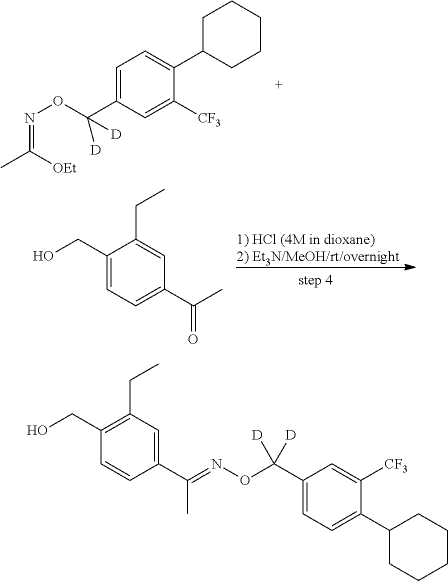

[4-[(1E)-1-([[4-cyclohexyl-3-(trifluoromethyl)phenyl](²H₂)methoxy]imino)ethyl]-2-ethylphenyl]methanol: To a solution of (Z)-(ethyl-N-[[4-cyclohexyl-3-(trifluoromethyl)phenyl](²H₂)methoxy]ethenecarboximidate) (800 mg, 2.32 mmol, 1.00 equiv.) in methanol (10 mL), hydrogen chloride (4M in dioxane) (1.2 mL, 0.50 equiv.) was added. The resulting solution was stirred for 30 minutes at room temperature. To this triethylamine (351 mg, 3.47 mmol, 1.50 equiv.) was added to adjust the pH value to 4-6, then the mixture was added a solution of 1-[3-ethyl-4-(hydroxymethyl) phenyl]ethan-1-one (412.8 mg, 2.32 mmol, 1.00 equiv.). The resulting solution was stirred overnight at room temperature. The resulting solution was diluted with 30 mL of water, extracted with ethyl acetate (3×50 mL) and the organic layers were combined, washed with brine (50 mL), dried over anhydrous sodium sulfate and concentrated under vacuum. The residue was purified by a silica gel column, eluted with ethyl acetate/petroleum ether (1:5-1:4) to afford 800 mg (79%) of [4-[(1E)-1-([[4-cyclohexyl-3-(trifluoromethyl)phenyl](²H₂)methoxy]imino)ethyl]-2-ethylphenyl] methanol as light yellow oil. LC-MS: m/z=436.15[M+H]⁺.

Step 5

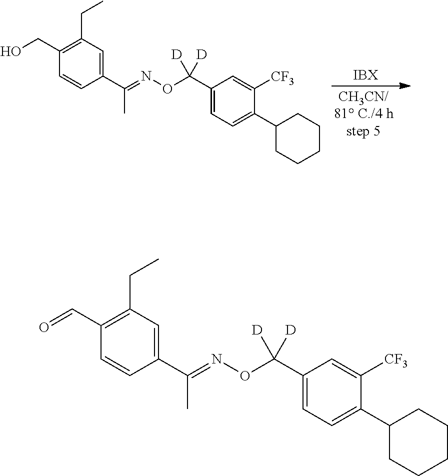 [SMILES: [2H]C([2H])(O/N=C(\C)c1ccc(CO)c(CC)c1)c1ccc(C2CCCCC2)c(C(F)(F)F)c1 ; [2H]C([2H])(O/N=C(\C)c1ccc(C=O)c(CC)c1)c1ccc(C2CCCCC2)c(C(F)(F)F)c1]

4-[(1E)-1-([[4-cyclohexyl-3-(trifluoromethyl)phenyl](²H₂)methoxy]imino)ethyl]-2-ethylbenzaldehyde: To a solution of [4-[(1E)-1-([[4-cyclohexyl-3-(trifluoromethyl)phenyl](²H₂)methoxy]imino)ethyl]-2-ethylphenyl]methanol (800 mg, 1.84 mmol, 1.00 equiv) in acetonitrile (10 mL), IBX (1.03 g, 3.68 mmol, 2.00 equiv.) was added. The resulting solution was stirred for 4 h at 81° C. The reaction mixture was cooled and concentrated under vacuum then diluted with ethyl acetate (50 mL). The solids were filtered out and concentrated under vacuum to afford 740 mg (93%) of 4-[(1E)-1-([[4-cyclohexyl-3-(trifluoromethyl)phenyl](²H₂)methoxy]imino)ethyl]-2-ethylbenzaldehyde as light yellow oil. LC-MS: m/z=434.15[M+H]⁺.

Step 6

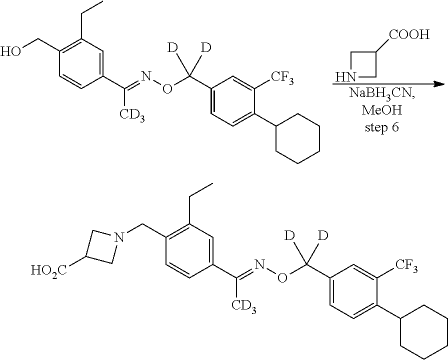 [SMILES: [2H]C([2H])([2H])/C(=N\OC([2H])([2H])c1ccc(C2CCCCC2)c(C(F)(F)F)c1)c1ccc(CO)c(CC)c1 ; O=C(O)C1CNC1 ; [2H]C([2H])([2H])/C(=N\OC([2H])([2H])c1ccc(C2CCCCC2)c(C(F)(F)F)c1)c1ccc(CN2CC(C(=O)O)C2)c(CC)c1]

1-([4-[(1E)-1-([[4-cyclohexyl-3-(trifluoromethyl)phenyl](²H₂)methoxy]imino)ethyl]-2-ethylphenyl]methyl)azetidine-3-carboxylic acid: To a solution of 4-[(1E)-1-([[4-cyclohexyl-3-(trifluoromethyl)phenyl](²H₂)methoxy] imino)ethyl]-2-ethylbenzaldehyde (500 mg, 1.15 mmol, 1.00 equiv.) in methanol (10 mL) azetidine-3-carboxylic acid (233.22 mg, 2.31 mmol, 2.00 equiv.) with acetic acid (0.7 mL) was added. The resulting solution was stirred for 20 min at room temperature. Then NaBH₃CN (36.24 mg, 0.58 mmol, 0.50 equiv.) was added in portions. The resulting solution was stirred overnight at room temperature. The resulting solution was diluted with water (10 mL). The mixture was extracted with ethyl acetate (3×30 mL), dried over anhydrous sodium sulfate, and concentrated under vacuum. The crude product was purified by Prep-HPLC with the following conditions: Column, XBridge Shield RP18 OBD Column, Sum, 19*150 mm; mobile phase, Water (0.1% FA) and ACN (35.0% ACN up to 55.0% in 7 min); Detector, UV 254 nm to afford 120 mg (20%) of 1-([4-[(1E)-1-([[4-cyclohexyl-3-(fluoromethyl)phenyl](²H₂)methoxy]imino) ethyl]-2-ethylphenyl]methyl)azetidine-3-carboxylic acid as a white solid. ¹H NMR (300 MHz, CD₃OD) δ: 7.66-7.51 (m, 5H), 7.41-7.38 (m, 1H), 4.45 (s, 2H), 4.24-4.21 (m, 4H), 3.45-3.40 (m, 1H), 2.95-2.88 (m, 1H), 2.79 (q, J=7.5 Hz, 2H), 2.24 (s, 3H), 1.86-1.75 (m, 5H), 1.57-1.36 (m, 5H), 1.23 (t, J=7.5 Hz, 3H). LC-MS: m/z=519.35 [M+H]⁺.

### Example 6: 1-([4-[(1E)-1-([[4-cyclohexyl-3-(trifluoromethyl)phenyl](²H₂)methoxy]imino)(²H₃)ethyl]-2-(²H₅)ethylphenyl](²H₂)methyl)azetidine-3-carboxylic acid

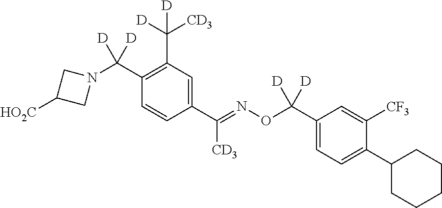 [SMILES: [2H]C([2H])([2H])/C(=N\OC([2H])([2H])c1ccc(C2CCCCC2)c(C(F)(F)F)c1)c1ccc(C([2H])([2H])N2CC(C(=O)O)C2)c(C([2H])([2H])C([2H])([2H])[2H])c1]

Step 1

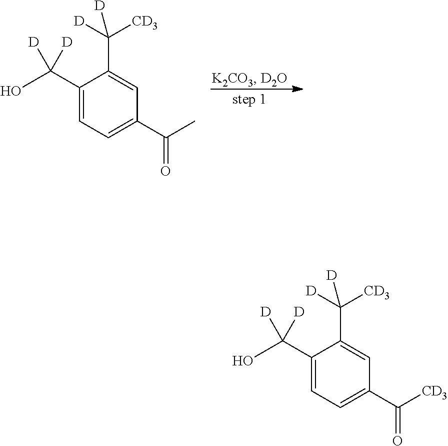 [SMILES: [2H]C([2H])(O)c1ccc(C(C)=O)cc1C([2H])([2H])C([2H])([2H])[2H] ; [2H]C([2H])([2H])C(=O)c1ccc(C([2H])([2H])O)c(C([2H])([2H])C([2H])([2H])[2H])c1]

1-[3-(²H₅)ethyl-4-[hydroxy(²H₂)methyl]phenyl](²H₃)ethan-1-one: To a solution of 1-[3-(²H₅)ethyl-4-[hydroxy(²H₂)methyl]phenyl]ethan-1-one (900 mg, 4.86 mmol, 1.00 equiv) in D₂O (10 mL), potassium carbonate (2.01 g, 14.54 mmol, 3.00 equiv) was added. The resulting solution was stirred for 15 h at 100° C. The reaction was monitored by LCMS. The resulting solution was extracted with ethyl acetate (3×10 mL), dried over anhydrous sodium sulfate, and concentrated under vacuum to afford 800 mg (87%) of 1-[3-(²H₅)ethyl-4-[hydroxy(²H₂)methyl]phenyl](²H₃)ethan-1-one as light yellow oil.

Step 2

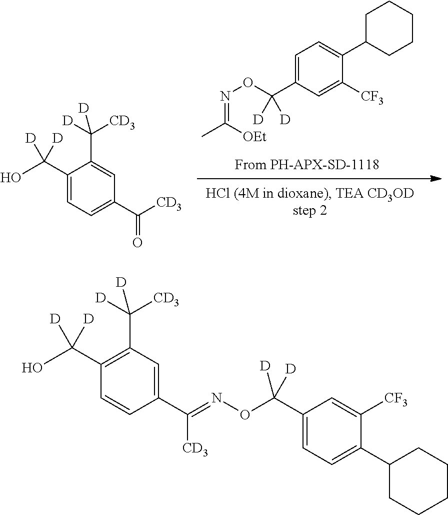 [SMILES: [2H]C([2H])([2H])C(=O)c1ccc(C([2H])([2H])O)c(C([2H])([2H])C([2H])([2H])[2H])c1 ; [2H]C([2H])(O/N=C(/C)OCC)c1ccc(C2CCCCC2)c(C(F)(F)F)c1 ; [2H]C([2H])([2H])/C(=N\OC([2H])([2H])c1ccc(C2CCCCC2)c(C(F)(F)F)c1)c1ccc(C([2H])([2H])O)c(C([2H])([2H])C([2H])([2H])[2H])c1]

[4-[(1E)-1-([[4-cyclohexyl-3-((rifluoromethyl)phenyl](²H₂) methoxy]imino) (²H₃)ethyl]-2-(²H₅)ethylphenyl](²H₂) methanol: To a solution of (Z)-(ethyl N-[[4-cyclohexyl-3-(trifluoromethyl)phenyl](²H₂)methoxy] ethenecarboximidate) (800 mg, 2.32 mmol, 1.00 equiv.) in CD₃OD (10 mL), hydrogen chloride(4M in dioxane) (1.16 mL) was added. The resulting solution was stirred for 30 min at room temperature. To this solution was added TEA (351 mg, 3.47 mmol, 1.50 equiv.) to adjust the pH value to 4-6. Then a solution of 1-[3-(²H₅)ethyl-4-[hydroxy(²H₂)methyl]phenyl](²H₃)ethan-1-one (436 mg, 2.32 mmol, 1.00 equiv.) in CD₃OD (2 mL) was added dropwise. The resulting solution was stirred overnight at room temperature. The resulting solution was concentrated under vacuum. The residue was purified by SiO₂ chromatography, eluted with ethyl acetate/petroleum ether (1:7) to afford 900 mg (87%) of [4-[(1E)-1-([[4-cyclohexyl-3-(trifluoromethyl)phenyl](²H₂)methoxy]imino)(²H₃)ethyl]-2-(²H₅)ethylphenyl](²H₂) methanol as light yellow oil. LC-MS: m/z=446 [M+H]⁺.

Step 3

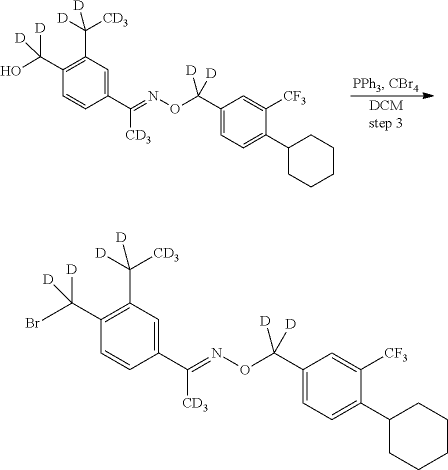 [SMILES: [2H]C([2H])([2H])/C(=N\OC([2H])([2H])c1ccc(C2CCCCC2)c(C(F)(F)F)c1)c1ccc(C([2H])([2H])O)c(C([2H])([2H])C([2H])([2H])[2H])c1 ; [2H]C([2H])([2H])/C(=N\OC([2H])([2H])c1ccc(C2CCCCC2)c(C(F)(F)F)c1)c1ccc(C([2H])([2H])Br)c(C([2H])([2H])C([2H])([2H])[2H])c1]

(E)-(1-[4-[bromo(²H₂)methyl]-3-(²H₅)ethylphenyl](²H₃)ethylidene)([[4-cyclohexyl-3-(trifluoromethyl)phenyl](²H₂)methoxy])amine: To a solution of [4-[(1E)-1-([[4-cyclohexyl-3-(trifluoromethyl)phenyl](²H₂)methoxy] imino)(²H₃)ethyl]-2-(²H₅)ethylphenyl](²H₂) methanol (650 mg, 1.46 mmol, 1.00 equiv.) in DCM (15 mL), PPh₃ (574 mg, 2.19 mmol, 1.50 equiv) and CBr₄ (725 mg, 1.50 equiv.) was added with stirring for 2 h at 0° C. The resulting solution was diluted with water (20 mL), extracted with DCM (3×30 mL) and the organic layers were combined. The resulting mixture was washed with brine (20 mL), dried over anhydrous sodium sulfate and concentrated under vacuum. The residue was purified by SiO₂ chromatography, eluted with ethyl acetate/petroleum ether (1:50) to afford 650 mg (88%) of (E)-(1-[4-[bromo(²H₂)methyl]-3-(²H₅)ethylphenyl](²H₃)ethylidene)([[4-cyclohexyl-3-(trifluoromethyl)phenyl](²H₂)methoxy])amine as light yellow oil. LC-MS: m/z=508 [M+H]⁺.

Step 4

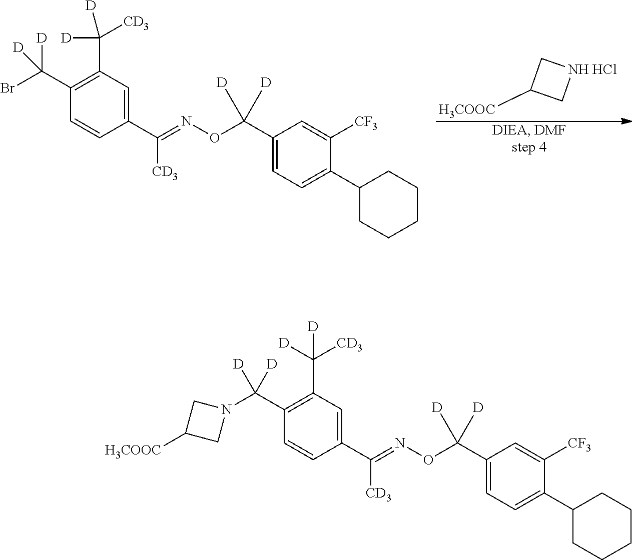

Methyl1-([4-[(1E)-1-([[4-cyclohexyl-3-(trifluoromethyl)phenyl](²H₂)methoxy]imino)(²H₃)ethyl]-2-(²H₅)ethylphenyl](²H₂)methyl) azetidine-3-carboxylate: To a solution of (E)-(1-[4-[bromo(²H₂)methyl]-3-(²H₅)ethylphenyl](²H₃)ethylidene)([[4-cyclohexyl-3-(trifluoromethyl)phenyl](²H₂)methoxy])amine (600 mg, 1.18 mmol, 1.00 equiv.) in DMF (10 mL) DIEA (458 mg, 3.54 mmol, 3.00 equiv.), methyl azetidine-3-carboxylate hydrochloride (268 mg, 1.77 mmol, 1.50 equiv) was added. The resulting solution was stirred for 3 h at room temperature. The resulting solution was diluted with water (30 mL), extracted with ethyl acetate (3×30 mL), and the organic layers were combined. The resulting mixture was washed with brine (20 mL), dried over anhydrous sodium sulfate, and concentrated under vacuum. The residue was purified by SiO₂ chromatography, eluted with ethyl acetate/petroleum ether (1:9) to afford 500 mg (78%) of Methyl1-([4-[(1E)-1-([[4-cyclohexyl-3-(trifluoromethyl)phenyl](²H₂)methoxy]imino)(²H₃)ethyl]-2-(²H₅)ethylphenyl](²H₂)methyl)azetidine-3-carboxylate as light yellow oil. LC-MS: m/z=543 [M+H]⁺.

Step 5

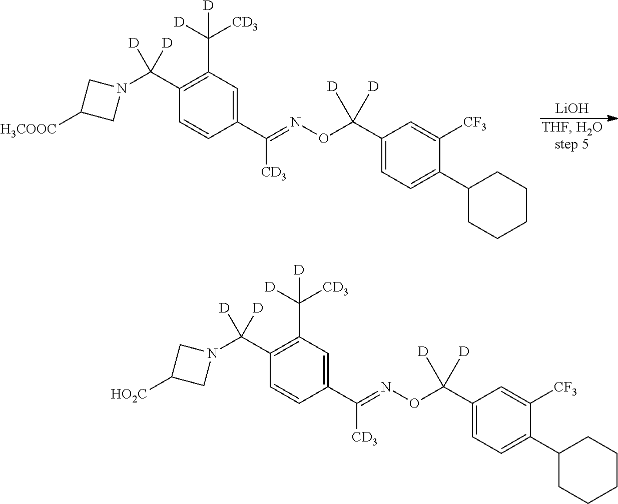

1-([4-[(1E)-1-([[4-cyclohexyl-3-(trifluoromethyl)phenyl](²H₂) methoxy]imino)(²H₃)ethyl]-2-(²H₅)ethylphenyl](²H₂)methyl)azetidine-3-carboxylic acid: To a solution of Methyl1-([4-[(1E)-1-([[4-cyclohexyl-3-(trifluoromethyl)phenyl](²H₂) methoxy]imino)(²H₃)ethyl]-2-(²H₅)ethylphenyl](²H₂)methyl)azetidine-3-carboxylate (500 mg, 0.92 mmol, 1.00 equiv.) in THF (9 mL) and water(3 mL), lithium hydroxide (66 mg, 2.76 mmol, 3.00 equiv.) was added. The resulting solution was stirred for 3 h at room temperature. Then the pH value of the mixture was adjusted to 3-5 with hydrochloric acid (1 M). The mixture was extracted with ethyl acetate (3×30 mL), dried over anhydrous sodium sulfate and concentrated under vacuum. The crude product was purified by Prep-HPLC with the following conditions: Column, XBridge Shield RP18 OBD Column, Sum, 19*150 mm; mobile phase, Water (0.1% FA) and ACN (35.0% ACN up to 55.0% in 7 min); Detector, UV 254 nm to afford 200 mg (41%) of 1-([4-[(1E)-1-([[4-cyclohexyl-3-(trifluoromethyl)phenyl](²H₂)methoxy]imino)(²H₃)ethyl]-2-(²H₅)ethylphenyl](²H₂)methyl)azetidine-3-carboxylic acid as a white solid. ¹H NMR (300 MHz, CD₃OD) δ: 7.66-7.51 (m, 5H), 7.41-5.38 (m, 1H), 4.24-4.22 (m, 4H), 3.49-3.31 (m, 1H), 2.95-2.88 (m, 1H) 1.87-1.75 (m, 5H) 1.58-1.31 (m, 5H). LC-MS: m/z=529 [M+H]⁺.

The following compounds can generally be made using the methods described above.

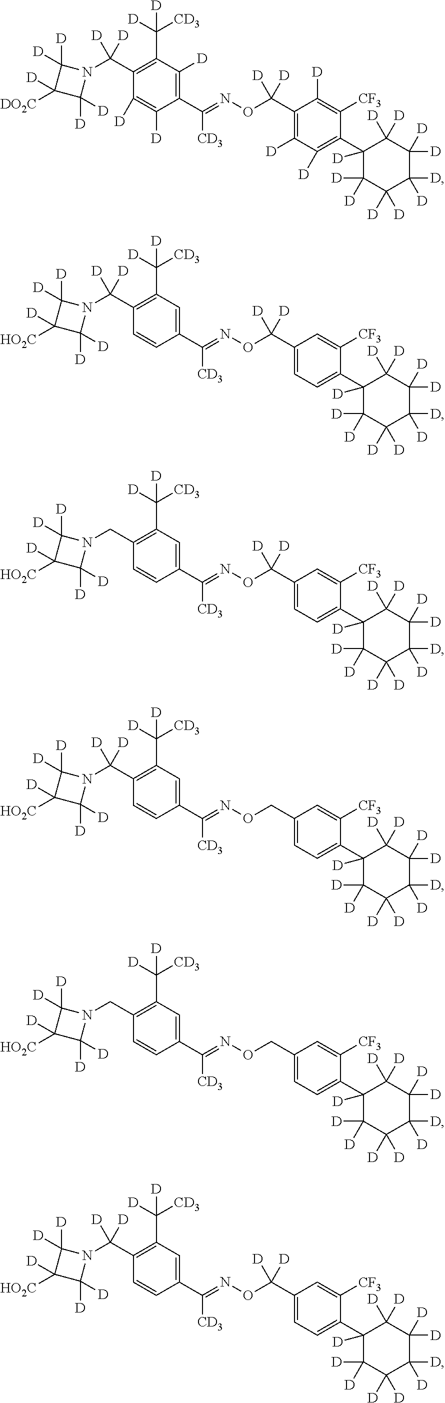

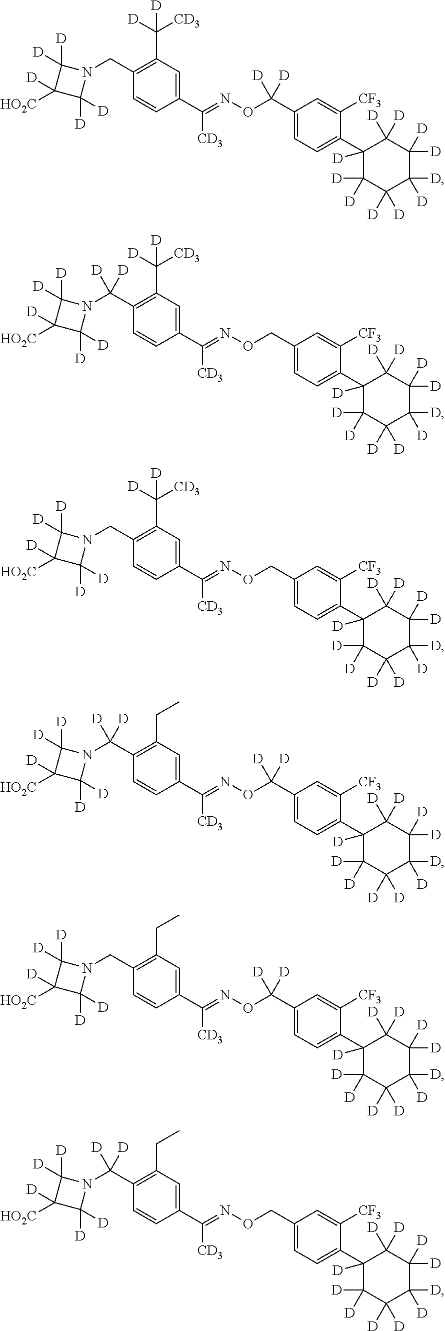

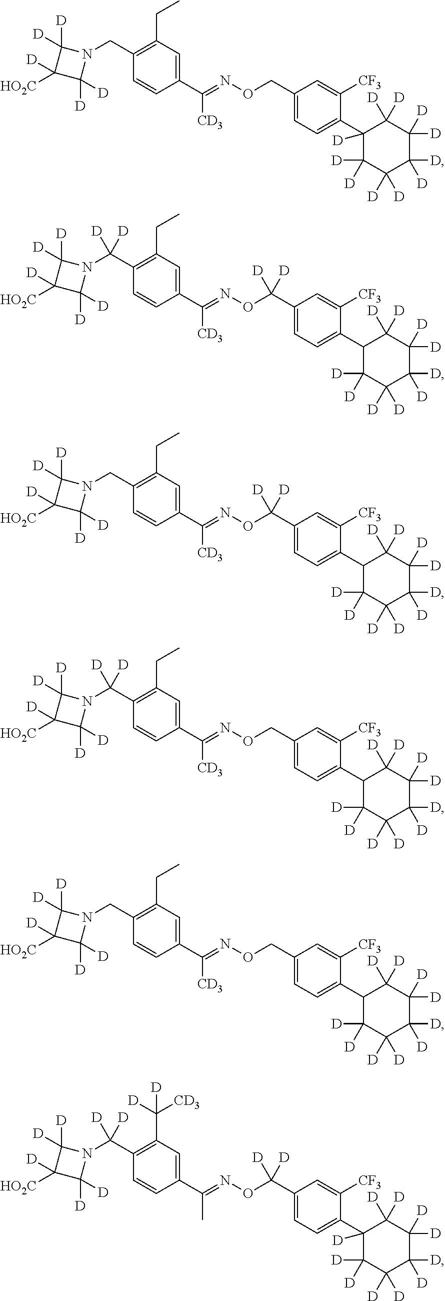

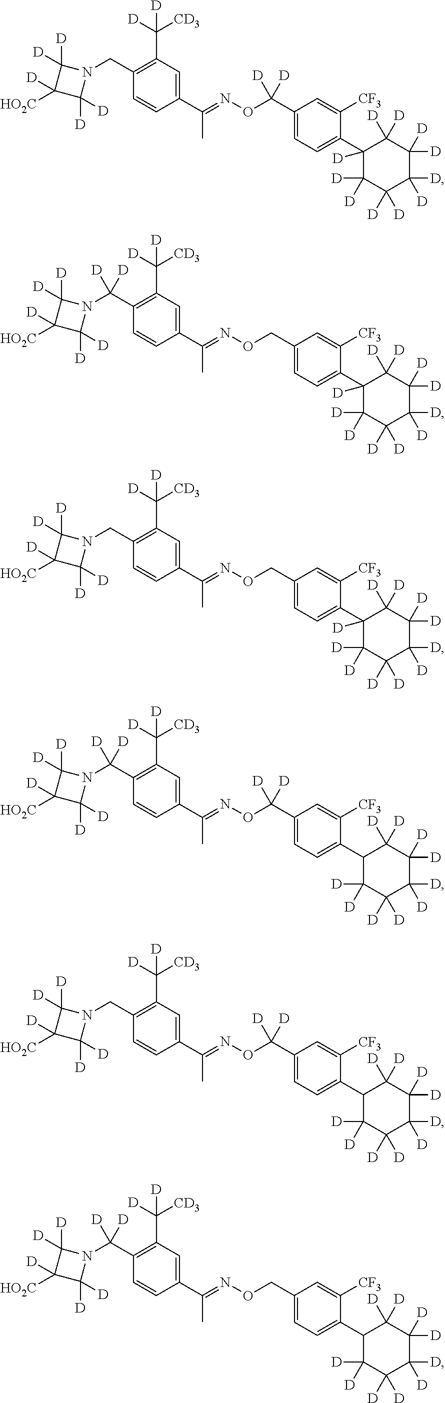

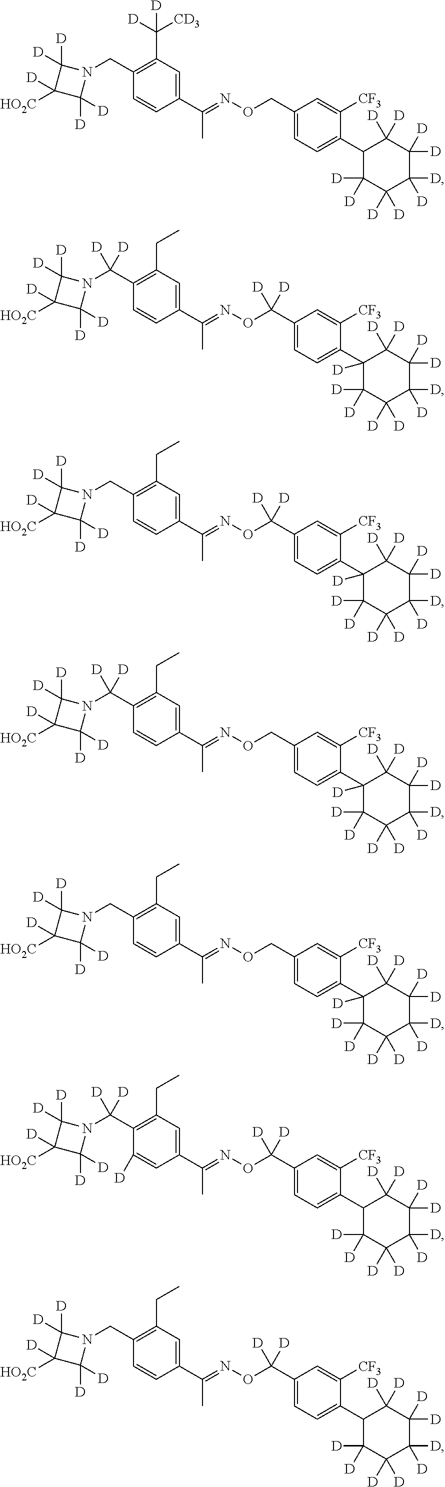

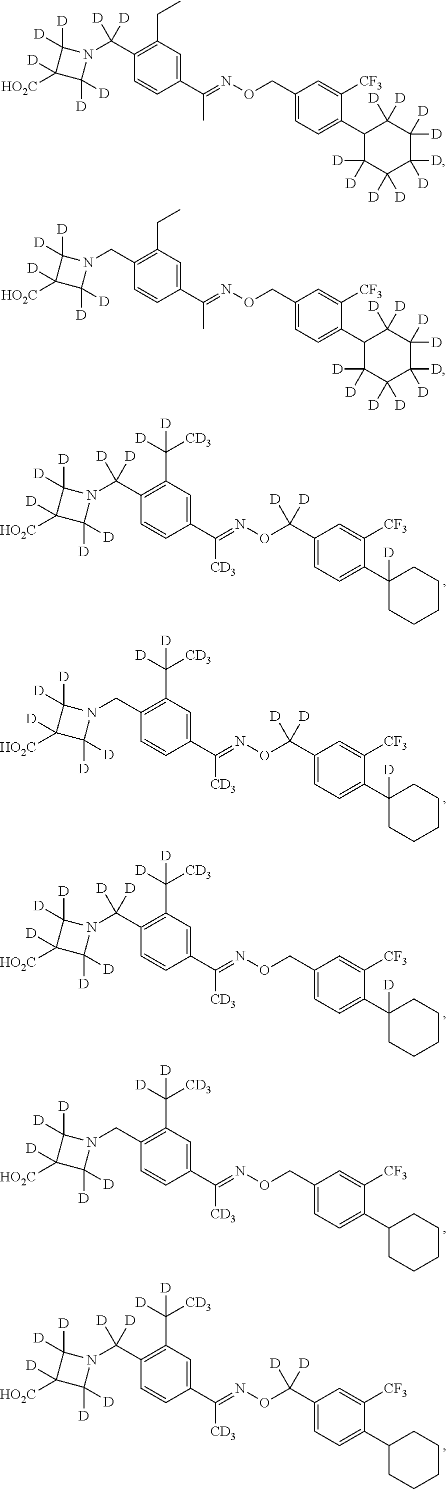

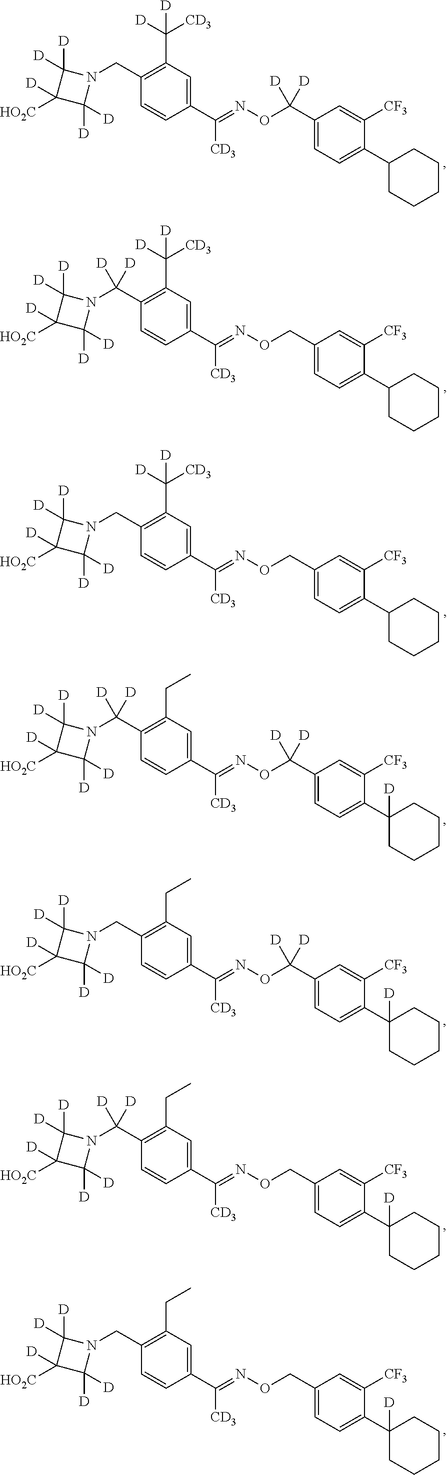

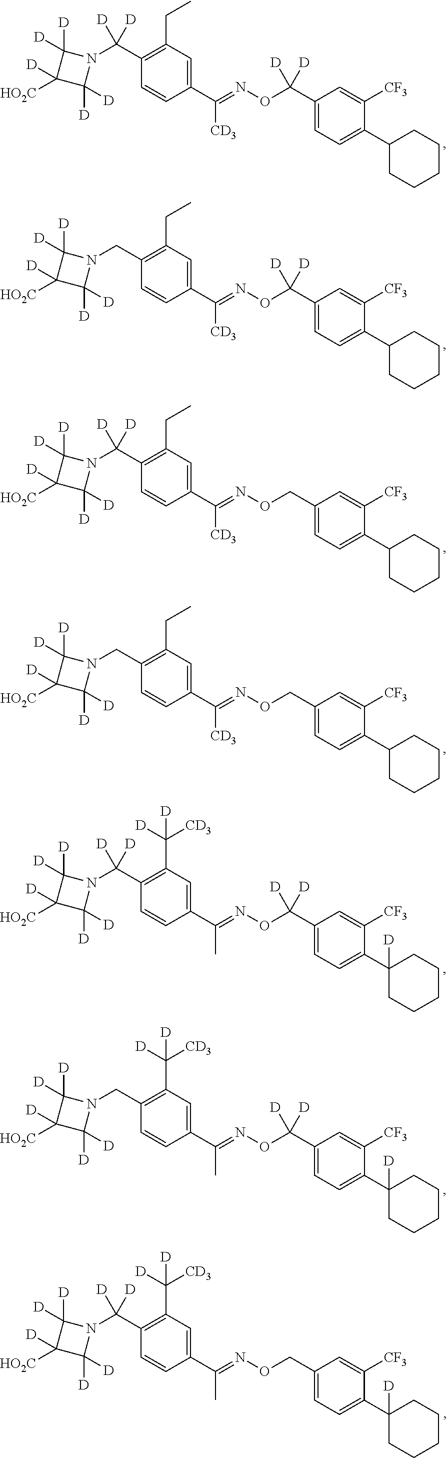

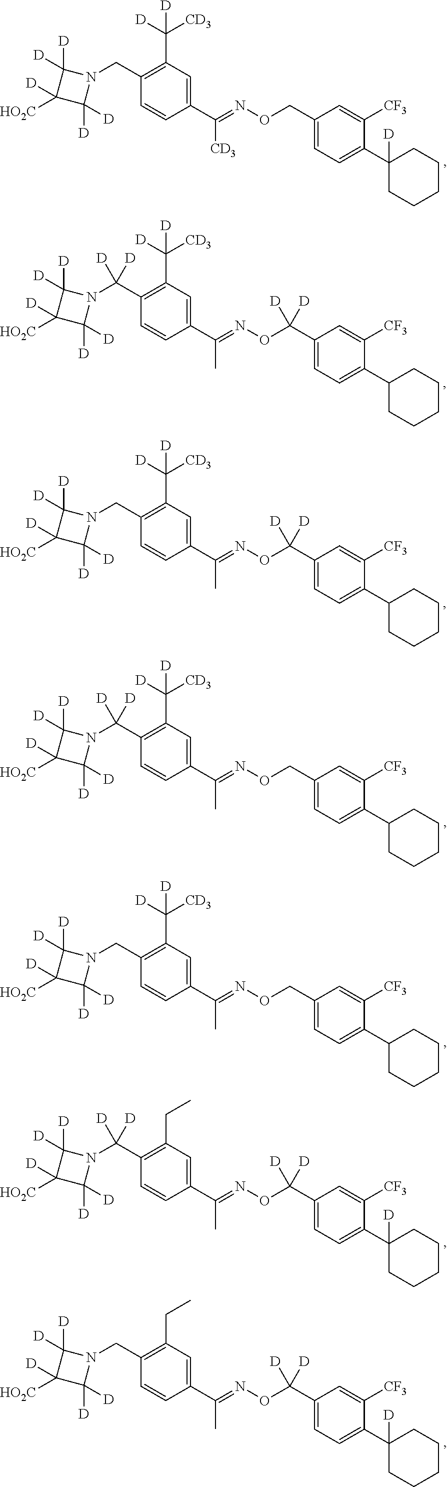

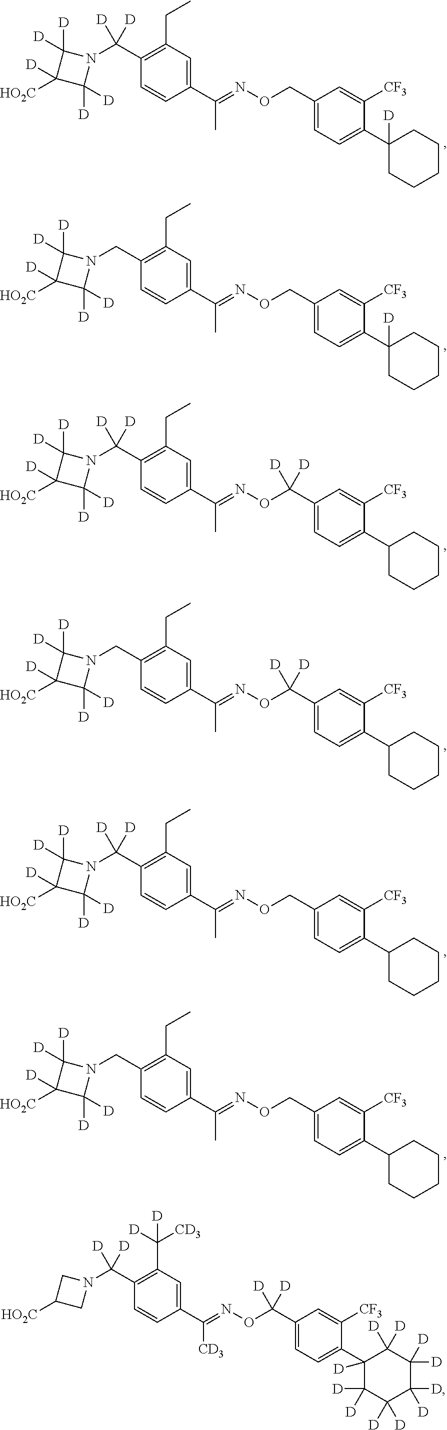

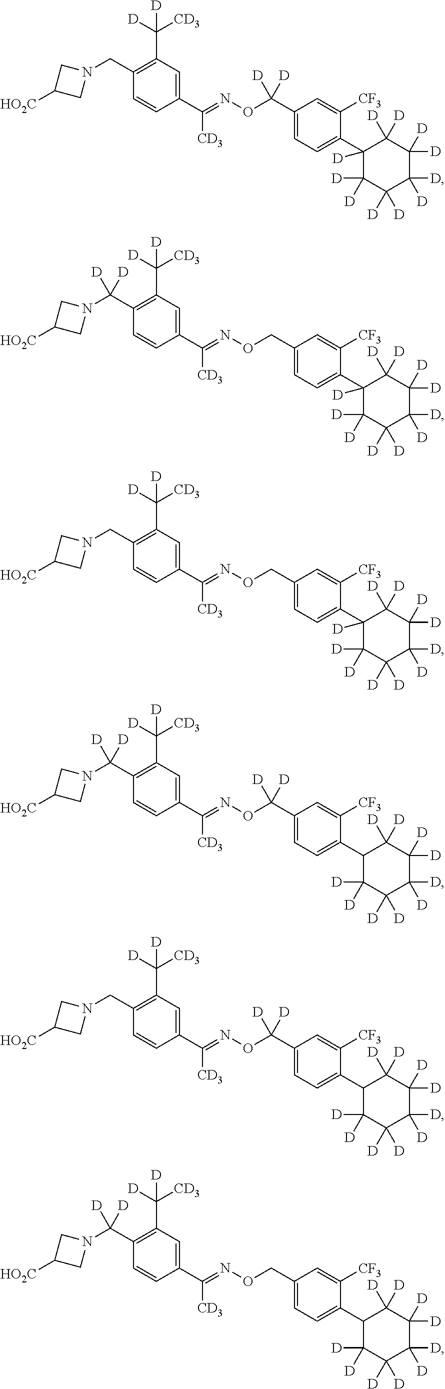

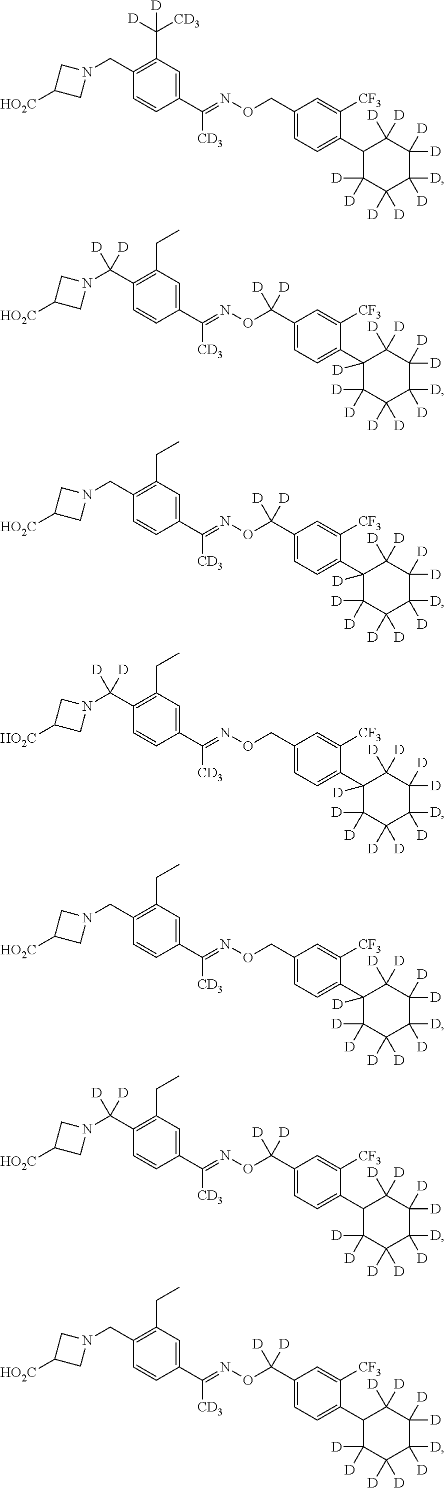

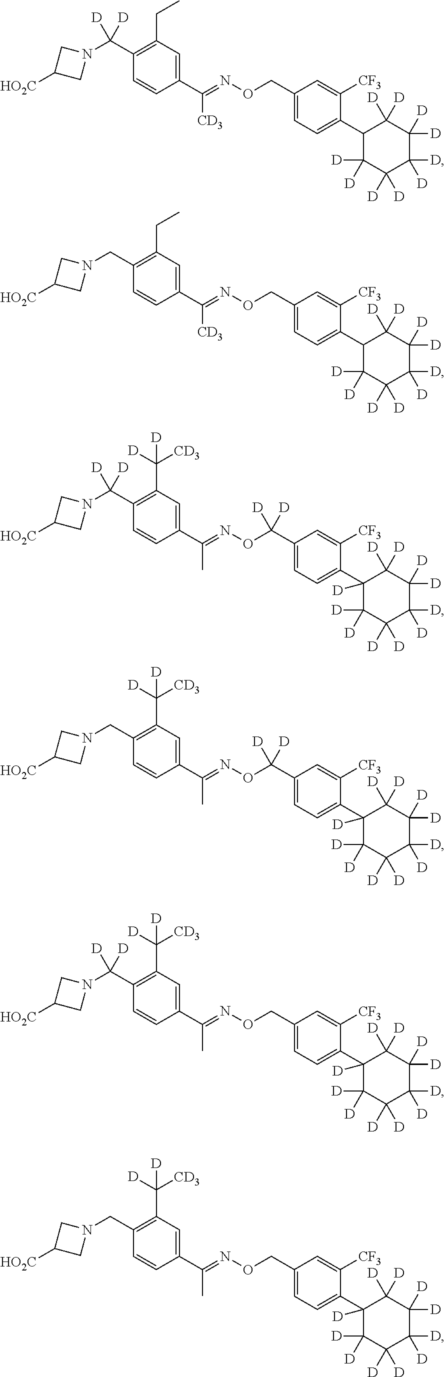

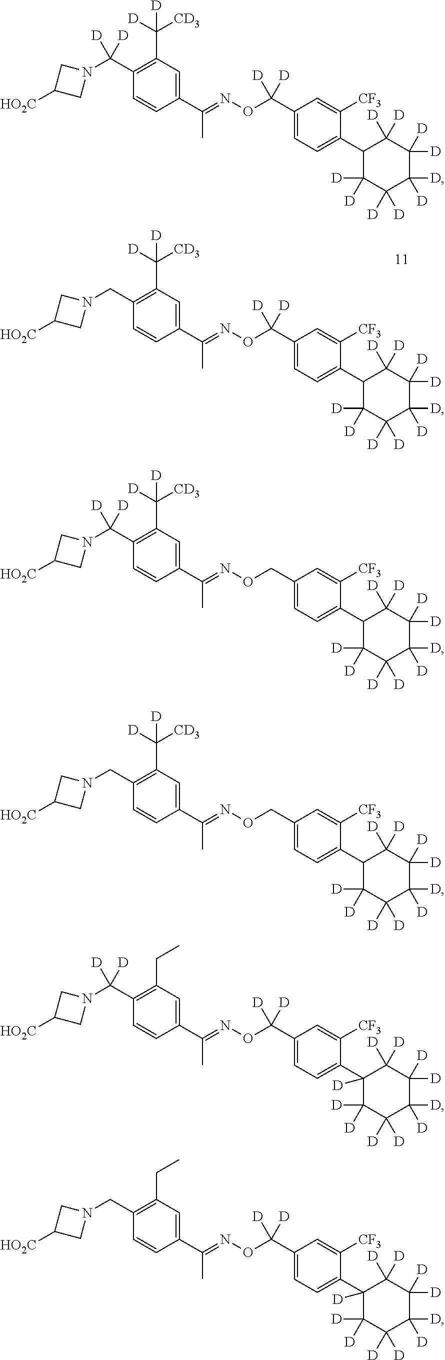

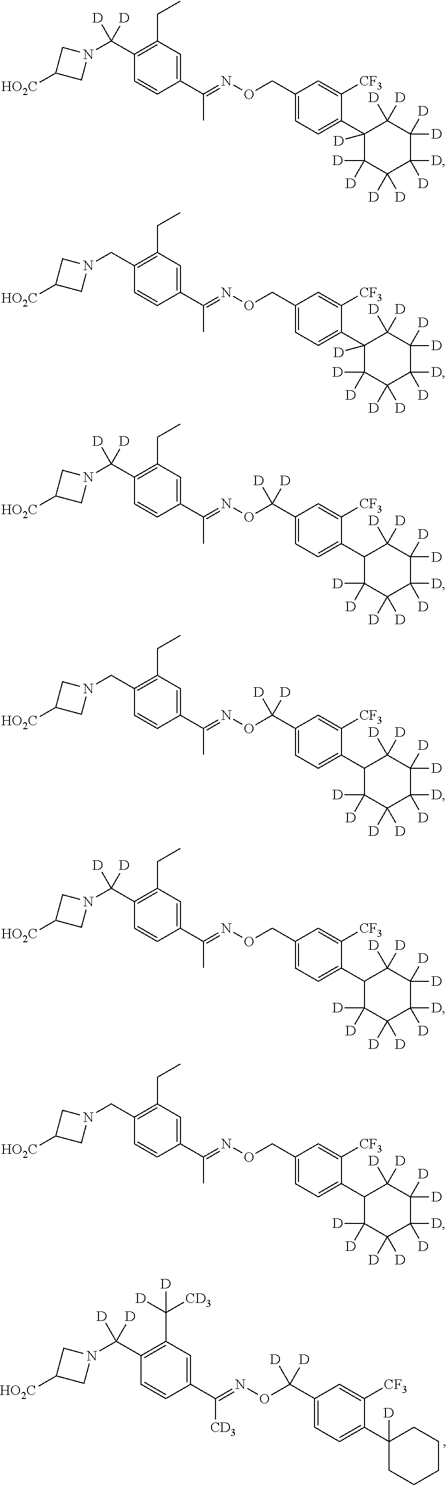

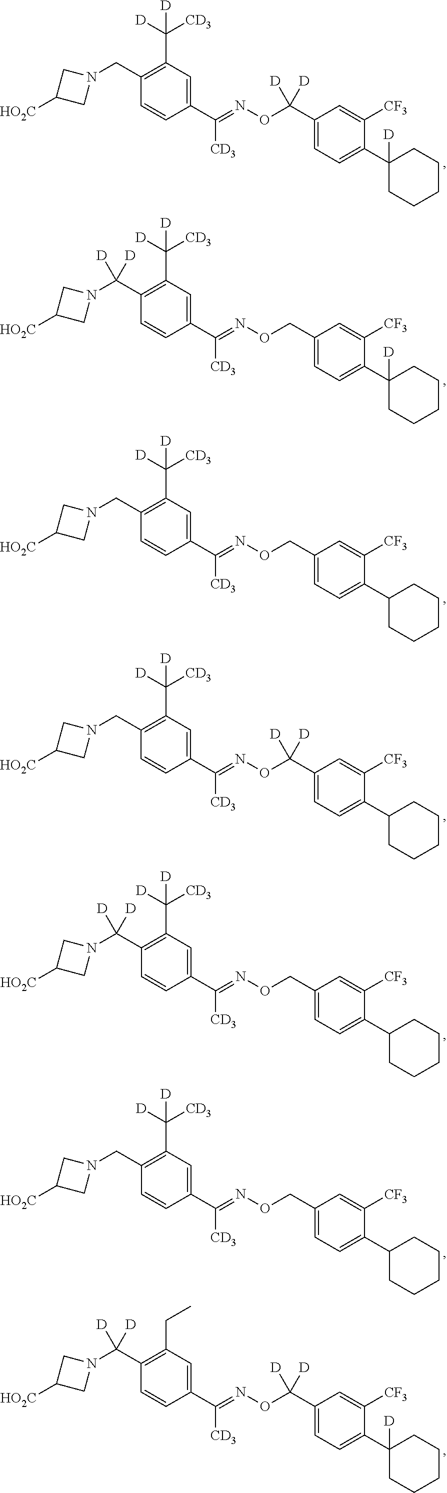

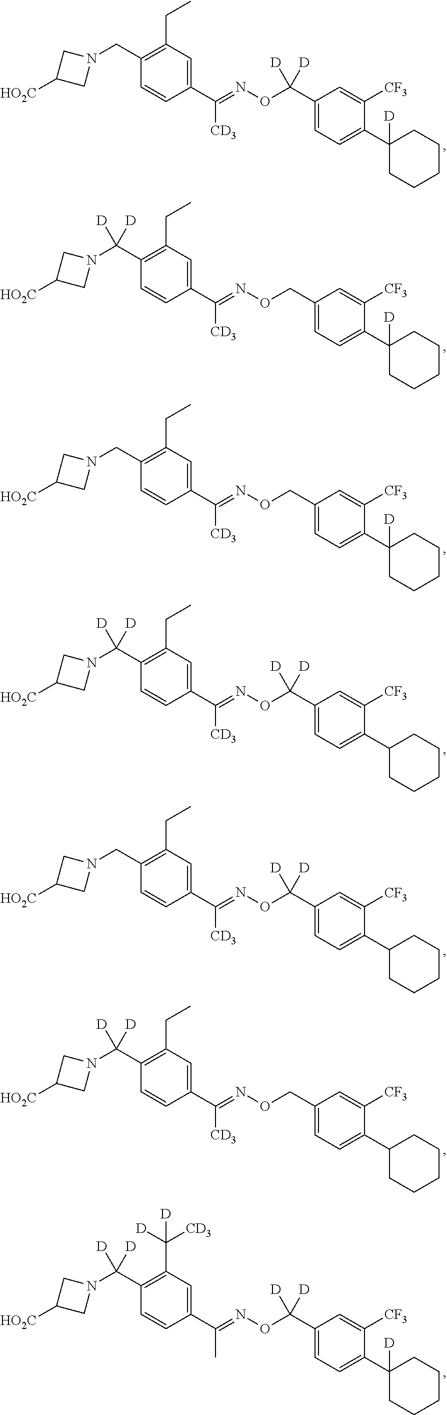

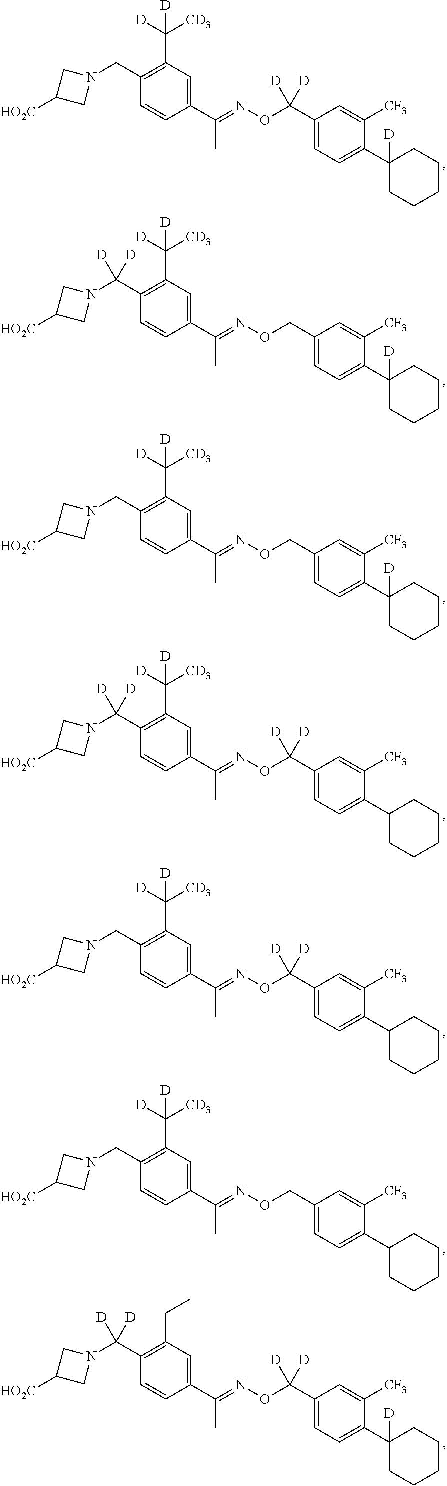

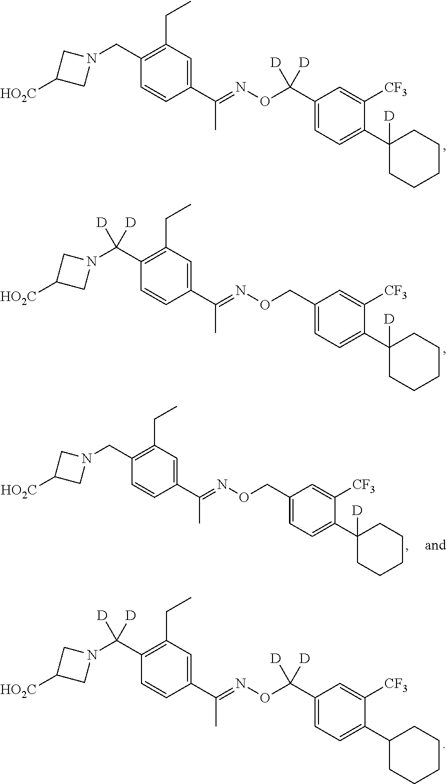 [SMILES: [2H]C1(c2ccc(C([2H])([2H])O/N=C(\C)c3ccc(CN4CC(C(=O)O)C4)c(CC)c3)cc2C(F)(F)F)CCCCC1 ; [2H]C1(c2ccc(CO/N=C(\C)c3ccc(C([2H])([2H])N4CC(C(=O)O)C4)c(CC)c3)cc2C(F)(F)F)CCCCC1 ; [2H]C1(c2ccc(CO/N=C(\C)c3ccc(CN4CC(C(=O)O)C4)c(CC)c3)cc2C(F)(F)F)CCCCC1 ; [2H]C([2H])(O/N=C(\C)c1ccc(C([2H])([2H])N2CC(C(=O)O)C2)c(CC)c1)c1ccc(C2CCCCC2)c(C(F)(F)F)c1]

Changes in the metabolic properties of the compounds disclosed herein as compared to their non-isotopically enriched analogs can be shown using the following assays. Compounds listed above which have not yet been made and/or tested are predicted to have changed metabolic properties as shown by one or more of these assays as well.

### Biological Activity Assays

### In Vitro Liver Microsomal Stability Assay

Human liver microsomal stability assays are conducted at 2 mg per mL liver microsome protein with an NADPH-generating system consisting of NADP (1 mM, pH 7.4), glucose-5-phosphate (5 mM, pH 7.4), and glucose-6-phosphate dehydrogenase (1 unit/mL). Test compounds are prepared as solutions in DMSO and added to the assay mixture (1 μM, final concentration in incubation) to be incubated at 37±1° C. Reactions are initiated with the addition of cofactor and were stopped at 0, 60, 120, or 240 min after cofactor addition with stop reagent (0.2 mL acetonitrile). Samples are centrifuged (920×g for 10 min at 10° C.) in 96-well plates. Supernatant fractions are analyzed by LC-MS/MS to determine the percent remaining and estimate the degradation half-life of the test compounds.

### In Vitro Metabolism Using Human Cytochrome P₄₅₀ Enzymes

The cytochrome P₄₅₀ enzymes are expressed from the corresponding human cDNA using a baculovirus expression system (BD Biosciences, San Jose, Calif.). A 0.25 milliliter reaction mixture containing 0.8 milligrams per milliliter protein, 1.3 millimolar NADP⁺, 3.3 millimolar glucose-6-phosphate, 0.4 U/mL glucose-6-phosphate dehydrogenase, 3.3 millimolar magnesium chloride and 0.2 millimolar of a compound of Formula I, the corresponding non-isotopically enriched compound or standard or control in 100 millimolar potassium phosphate (pH 7.4) is incubated at 37° C. for 20 min. After incubation, the reaction is stopped by the addition of an appropriate solvent (e.g., acetonitrile, 20% trichloroacetic acid, 94% acetonitrile/6% glacial acetic acid, 70% perchloric acid, 94% acetonitrile/6% glacial acetic acid) and centrifuged (10,000 g) for 3 min. The supernatant is analyzed by HPLC/MS/MS.

| Cytochrome P₄₅₀ | Standard |
|---|---|
| CYP1A2 | Phenacetin |
| CYP2A6 | Coumarin |
| CYP2B6 | [¹³C]-(S)-mephenytoin |
| CYP2C8 | Paclitaxel |
| CYP2C9 | Diclofenac |
| CYP2C19 | [¹³C]-(S)-mephenytoin |
| CYP2D6 | (+/−)-Bufuralol |
| CYP2E1 | Chlorzoxazone |
| CYP3A4 | Testosterone |
| CYP4A | [¹³C]-Lauric acid |

### Monoamine Oxidase a Inhibition and Oxidative Turnover

The procedure is carried out using the methods described by Weyler, Journal of Biological Chemistry 1985, 260, 13199-13207, which is hereby incorporated by reference in its entirety. Monoamine oxidase A activity is measured spectrophotometrically by monitoring the increase in absorbance at 314 nm on oxidation of kynuramine with formation of 4-hydroxyquinoline. The measurements are carried out, at 30° C., in 50 mM NaPᵢ buffer, pH 7.2, containing 0.2% Triton X-100 (monoamine oxidase assay buffer), plus 1 mM kynuramine, and the desired amount of enzyme in 1 mL total volume.

### Monooamine Oxidase B Inhibition and Oxidative Turnover

The procedure is carried out as described in Uebelhack, Pharmacopsychiatry 1998, 31(5), 187-192, which is hereby incorporated by reference in its entirety.

### Experimental Autoimmune Neuritis

The procedure is carried out as described in WO 2010020610, which is hereby incorporated by reference in its entirety. S1P modulators have demonstrated utility in preventing experimental autoimmune neuritis; see, e.g., Zhang Z., 2009 J Neuroimmunol 216:59-65, which is hereby incorporated by reference in its entirety.

Male Lewis rats (8-10 weeks, 180-200 g, Elevage-Janvier, France) are housed under a 12 h light-12 h dark cycle and with free access to food and water. All animal procedures are in accordance with a protocol approved by the local Administration District Official Committee. All efforts are made to minimize the number of animals and their suffering.

EAN induction. For EAN induction, rats are immunized by subcutaneous injection into both hind footpads with 100 μl of an inoculum containing 100 μg of synthetic neuritogenic P2 57-81 peptide (GeneScript Corporation, Scotch Plains, N.J., USA). The peptide is dissolved in phosphate buffered saline (PBS) (2 mg/mL) and then emulsified with an equal volume of complete Freund's adjuvant (CFA) containing 2 mg/mL mycobacterium tuberculosis to get a final concentration of 1 mg/mL.

EAN clinical scores are evaluated every day as follows: 0=normal, 1=reduced tonus of tail, 2=limp tail, impaired righting, 3=absent righting, 4=gait ataxia, 5=mild paresis of the hind limbs, 6=moderate paraparesis, 7=severe paraparesis or paraplegia of the hind limbs, 8=tetraparesis, 9=moribund, and 10=death (Zhang et al., 2009A).

Compound treatment. Test compound is separately tested at two concentrations of suspension (3 and 10 mg/kg, suspended in 1% carboxymethylcellulose (CMC, Blanose, Hercules-Aqualon, Düsseldorf, Germany)). The compound suspensions are intragastrically administrated immediately after induction and then once daily until Day 22 (5 rats per group). For control EAN rats, the same volume of 1% CMC in water is given.

Immunohistochemistry. To evaluate inflammatory cell infiltration and pathological changes in the PNS, five compound A-treated rats (at both concentrations) and five control EAN rats from Day 16 are sacrificed. Rats are deeply anaesthetized with ether and perfused intracardially with 4° C., 4% paraformaldehyde in PBS. Left and right sciatic nerves are quickly removed and post-fixed in 4% formaldehyde overnight at 4° C. Sciatic nerves are cut into two equally long segments, embedded in paraffin, serially sectioned (3 pin) and mounted on silane-covered slides.

After dewaxing, cross-sections of sciatic nerves are boiled (in a 600 W microwave oven) for 15 min in citrate buffer (2.1 g sodium citrate/L, pH 6). Endogenous peroxidase is inhibited with 1% H₂O₂ in methanol for 15 min. Sections are incubated with 10% normal pig serum (Biochrom, Berlin, Germany) to block non-specific binding of immunoglobulins and then with the following monoclonal antibodies: W3/13 (1:50; Serotec, Oxford, UK) for T lymphocytes, 0X22 (1:200; Serotec, Oxford, UK) for B cells, ED1 for activated macrophages (1:100; Serotec, Oxford, UK). Antibody binding to tissue sections is visualized with biotinylated IgG F(ab)2 secondary antibody fragments (rabbit anti-mouse or rabbit anti-goat; 1:400; DAKO, Hamburg, Germany). Subsequently, sections are incubated with a Streptavidin-Avidin-Biotin complex (DAKO, Hamburg, Germany), followed by development with diaminobenzidine (DAB) substrate (Fluka, Neu-Ulm, Germany). Finally, sections are counterstained with Maier's Hemalum.

To evaluate immunostaining data, the percentages of areas of immunoreactivity (IR) to areas of sciatic nerve cross-sections are calculated. Images of sciatic nerve cross-sections are captured under 50^{χ} magnification using Nikon Coolscope (Nikon, Dusseldorf, Germany) with fixed parameters. Images are analyzed using MetaMorph Offline 7.1 (Molecular Devices, Toronto, Canada). Areas of IR are selected by colour threshold segmentation and all parameters are fixed for all images. Areas of sciatic nerve cross-sections are manually selected. For each EAN rat, four cross-sections from root and middle levels of both sides are analyzed. Results are given as arithmetic means of percentages of areas of IR to areas of sciatic nerve cross-sections and standard errors of means (SEM). The routine Luxol Fast Blue (LFB) staining is applied to show myelin. Histological changes between Compound A and control EAN rats are compared by an established semi-quantitative method. Briefly, four cross-sections from root and middle level of both sides of EAN rats are analyzed. All perivascular areas present in cross-sections are evaluated by two observers unaware of the treatment, and the degree of pathological alteration is graded semiquantitatively on the following scale: 0=normal perivascular area; 1=mild cellular infiltrate adjacent to the vessel; 2=cellular infiltration plus demyelination in immediate proximity to the vessel; and 3=cellular infiltration and demyelination throughout the section. Results are given as mean histological score (Hartung et al., 1988).

Evaluation and statistical analysis. The unpaired t-test is performed to compare difference between test compound and control EAN rats (Graph Pad Prism 4.0 for windows). For all statistical analyses, significance levels are set at p<0.05. Compounds disclosed herein may treat and/or prevent the development and/or symptoms of EAN in this assay, indicating that they are useful for the treatment of inflammatory, immune-mediated, and/or demyelinating peripheral neuropathies.

### Effect on Cytokine-Induced Atrophy of Primary Human Myotubes

The procedure is carried out as described in WO 2010010127, which is hereby incorporated by reference in its entirety. Human skeletal muscle (skMC) cells are obtained from Cambrex (#CC-2561). For experiments skMC stocks are thawed and maintained in skeletal muscle basal medium SkBM (Lonza CC-3161) containing 20% FCS and 0.1% gentamycin at 37° C., 5% CO₂. After 4-5 days cells are seeded for experiments onto six-well plates coated with matrigel (450,000/well) and grown at 37° C., 5% CO₂ for one day. Cells are then washed 3× with SkBM and differentiated into myotubes with SkBM containing 1 μM SB431542 (ALK4/5 inhibitor; Sigma #S4317) for 4 days (SB431542 is removed 24h prior cell treatment). Myotubes are then treated with test compound either in the absence or in the presence of a cytokine cocktail (TNFa 10 ng/ml, IL-1 β 2 ng/ml, IFNy 10 ng/ml) for 24h in SkBM plus 0.1% gentamycin, washed once with cytokine stabilization (CSB) buffer: 80 mM piperazine-N,N′-bis(2-ethanesulfonic acid (PIPES) buffer, 5 nM EGTA, 1 mM MgCl 6H₂O and 4% PEG35000 (Fluka #94646) and fixed with 4% paraformaldehyde (Electron Microscopy Sciences #15714) in CSB for 15 min at room temperature. Cells are then rinsed with CSB, permeabilized with 0.2% Triton X-100 (Merck #1.12298.0100) for 20 min at room temperature, rinsed with CSB and blocked with 10% normal goat serum blocking solution (Zymed Laboratories #50-062Z) for 20 min at room temperature. Primary antibody (anti-myosin heavy chain antibody; Upstate #05-716) diluted 1:500 in PBS containing 1.5% goat serum is added for 1 h at room temperature. Myotubes are then washed 2× with CSB (5 min/wash) then add secondary antibody (Alexa Fluor 488 F(ab′); Invitrogen #A1 1017) diluted 1:750 in PBS is added for 1 h at room temperature. Myotubes are washed once with CSB (10 min) then with PBS (Invitrogen #14190) and double distilled water. Finally, ProLong Gold antifade reagent with DAPI (Invitrogen #P36931) is added and myotubes are photographed. Average diameters of at least 40 myotubes are measured for each condition at three points separated by 50 μm along the length of the myotube. Human primary myoblasts, differentiated for 4 days and treated with cytokine cocktail for 24 h, fixed, and assayed for changes in myotube diameters show distinct atrophic phenotype with a decrease in myotube diameter as compared to vehicle control. Compounds disclosed herein may block cytokine-induced atrophy in human primary myoblasts. Compounds disclosed herein may have an inhibitory effect on inflammatory myopathies, e.g. polymyositis; to prevent, inhibit or treat muscle inflammation or inflammatory myopathies, e.g. polymyositis; to reduce or prevent or alleviate relapses in an inflammatory myopathy, e.g. polymyositis; and/or to slow progression of an inflammatory myopathy, e.g. polymyositis.

### In Vivo Anti-Angiogenesis Assay

The procedure is carried out as described in WO 2004103306, which is hereby incorporated by reference in its entirety. Porous chambers containing (i) sphingosine-1-phosphate (5 μM/chamber) or (ii) human VEGF (1 μg/chamber) in 0.5 ml of 0.8% w/v agar (containing heparin, 20 U/ml) are implanted subcutaneously in the flank of mice. S1P or VEGF induces the growth of vascularized tissue around the chamber. This response is dose-dependent and can be quantified by measuring the weight and blood content of the tissue. Mice are treated once a day orally or intravenously with a compound as disclosed herein starting 4-6 hours before implantation of the chambers and continuing for 4 days. The animals are sacrificed for measurement of the vascularized tissues 24 hours after the last dose. The weight and blood content of the vascularized tissues around the chamber is determined. Compounds disclosed herein may reduce weight and/or blood content of the vascularized tissues in treated animals compared to control.

### In Vitro Antitumor Assay

The procedure is carried out as described in WO 2004103306, which is hereby incorporated by reference in its entirety. A mouse breast cancer cell line originally isolated from mammary carcinomas is used, e.g. JygMC(A). The cell number is adjusted to 5×10⁵ for plating in fresh medium before the procedure. Cells are incubated with fresh medium containing 2.5 mM of thymidine without FCS for 12 hours and then washed twice with PBS, followed by addition of fresh medium with 10% FCS and additionally incubated for another 12 hours. Thereafter the cells are incubated with fresh medium containing 2.5 mM of thymidine without FCS for 12 hours. To release the cells from the block, the cells are washed twice with PBS and replated in fresh medium with 10% FCS. After synchronization, the cells are incubated with or without various concentrations of a compound as disclosed herein for 3, 6, 9, 12, 18 or 24 hours. The cells are harvested after treatment with 0.2% EDTA, fixed with ice-cold 70% ethanol solution, hydrolyzed with 250 μg/ml of RNaseA (type 1-A: Sigma Chem. Co.) at 37° C. for 30 minutes and stained with propidium iodide at 10 mg/ml for 20 minutes. After the incubation period, the number of cells is determined both by counting cells in a Coulter counter and by the SRB colorimetric assay. Compounds disclosed herein may inhibit the proliferation of the tumor cells in this assay.

### In Vitro Human EDG Receptor Assay

The procedure is carried out as described in WO 2004103306, which is hereby incorporated by reference in its entirety.

EDG-1 (SIP]) GTP [γ-³⁵S] binding assay: Homogenized membranes are prepared from CHO cell clones stably expressing a human EDG-1 N-terminal c-myc tag. Cells are grown in suspension in two 850 cm² roller bottles for three or four days before harvesting. The cells are centrifuged down, washed once with cold PBS, and resuspended in 20 ml of Buffer A (20 mM HEPES, pH 7.4, 10 mM EDTA, EDTA-free complete protease inhibitor cocktail [1 tablet/25 ml]). The cell suspension is homogenized on ice, using a Polytron homogenizer at 30000 rpm at three intervals of 15 seconds each. The homogenate is first centrifuged at 2000 rpm on a tabletop low speed centrifuge for 10 minutes. The supernatant, after passing through a cell strainer, is then re-centrifuged at 50,000×g for 25 minutes at 4° C. The pellet is resuspended into buffer B (15% glycerol, 20 mM HEPES, pH 7.4, 0.1 mM EDTA, EDTA-free complete protease inhibitor cocktail [1 tablet/10 ml]). Protein concentration of the prep is determined using the BCA Protein Assay kit (Pierce) using BSA as standard. The membranes are aliquoted and kept frozen at −80° C.

Solutions of test compounds, e.g., ranging from 10 mM to 0.01 nM, may be prepared in DMSO. S1P is diluted in 4% BSA solution as positive controls. The desired amount of membrane prep is diluted with ice-cold assay buffer (20 mM HEPES, pH 7.4, 100 mM NaCl, 10 mM MgCl₂, 0.1% Fatty acid-free BSA, 5 μM GDP) and vortexed well. 2 μl or less of compound is distributed into each well of a round-bottom 96-well polystyrene assay plate, followed by addition of 100 μl of diluted membranes (3-10 μg/well) and kept on ice until the addition of hot GTPγS. [³⁵S]-GTPγS is diluted 1:1000 (v/v) with cold assay buffer and 100 μl is added into each well. The reaction is carried out at room temperature for 90 minutes before the membranes are harvested onto Perkin-Elmer Unifilter® GF/B-96 filter plate using a Packard Filtermate Harvester. After several washes with wash buffer (20 mM HEPES, pH 7.4, 100 mM NaCl, 10 mM MgCl₂), and a rinse with 95% ethanol, the filter is dried in a 37° C. oven for 30 minutes. MicroScint-20 is added and the plate sealed for scintillation counting on TopCount. EC50 values are obtained by fitting the GTP [γ-³⁵S] binding curves (raw data) with the dose response curve-fitting tool of GraphPad Prism. Six or twelve different concentrations may be used to generate a concentration response curve (using three data points per concentration).

EDG-3, -5, -6 and -8 GTP [γ-³⁵S] binding assays may be carried out in a comparable manner to the EDG-1 GTP [γ-³⁵S] binding assay using membranes from CHO cells stably expressing c-terminal c-myc tagged or untagged receptors. For each membrane preparation, titration experiments are first run with S1P control to determine the optimal amount of membranes to be added per assay well. Compounds disclosed herein may have activity in this assay; in certain embodiments, compounds disclosed herein may be selective for EDG-1 compared to one or more of the other receptors including EDG-3, EDG-5, EDG-6 and EDG-8.

### In Vitro FLIPR Calcium Flux Assay

The procedure is carried out as described in WO 2004103306, which is hereby incorporated by reference in its entirety. Compounds of the disclosure are tested for agonist activity on EDG-1, EDG-3, EDG-5, and EDG-6 with a FLIPR calcium flux assay. Briefly, CHO cells expressing an EDG receptor are maintained in F-12K medium (ATCC), containing 5% FBS, with 500 ug/ml of G418. Prior to the assay, the cells are plated in 384 black clear bottom plates at the density of 10,000 cells/well/25 μl in the medium of F-12K containing 1% FBS. The second day, the cells are washed three times (25 μl/each) with washing buffer. About 25 μl of dye are added to each well and incubated for 1 hour at 37° C. and 5% CO₂. The cells are then washed four times with washing buffer (25 μl/each). The calcium flux is assayed after adding 25 μl of SEQ2871 solution to each well of cells. The same assay is performed with cells expressing each of the different EDG receptors. Titration in the FLIPR calcium flux assay is recorded over a 3-minute interval, and quantitated as maximal peak height percentage response relative to EDG-1 activation. Compounds disclosed herein may be characterized for agonist activity on EDG receptors including EDG-1, EDG-3, EDG-5, and EDG-6 using this assay, and may be EDG-1 agonists.

### In Vivo Assay for Blood Lymphocyte Depletion and Assessment of Heart Effect

The procedure is carried out as described in WO 2004103306, which is hereby incorporated by reference in its entirety.

Measurement of circulating lymphocytes: Compounds are dissolved in DMSO and diluted to obtain a final concentration of 4% DMSO (v/v, final concentration) and then further diluted in a constant volume of Tween80 25%/H₂O, v/v. Tween80 25%/H₂O (200 μl), 4% DMSO, and FTY720 (10 μg) are included as negative and positive controls, respectively. Mice (C57b1/6 male, 6-10 week-old) are administered, e.g. at 250-300 μL of compound solution orally by gavages under short isoflurane anesthesia.

Blood is collected from the retro-orbital sinus 6 and 24 hours after drug administration under short isoflurane anesthesia. Whole blood samples are subjected to hematology analysis. Peripheral lymphocyte counts are determined using an automated analyzer. Subpopulations of peripheral blood lymphocytes are stained by fluorochrome-conjugated specific antibodies and analyzed using a fluorescent activating cell sorter (Facscalibur). Two mice are used to assess the lymphocyte depletion activity of each compound screened. The result is an ED₅₀, which is defined as the effective dose required displaying 50% of blood lymphocyte depletion.

Assessment of Heart Effect: The effects of compounds on cardiac function are monitored using the AnonyMOUSE ECG screening system. Electrocardiograms are recorded in conscious mice (C57b1/6 male, 6-10 week-old) before and after compound administration. ECG signals are then processed and analyzed using the e-MOUSE software. Compound (e.g., 90 μg of further diluted in water, e.g. 200A and 15% DMSO) are injected IP. Four mice may be used to assess the heart effect of each compound. Compounds disclosed herein may reduce circulating lymphocytes in this assay; in certain embodiments, exhibit an ED₅₀ of less than 1 mg/kg, more preferably an ED₅₀ of less than 0.5 mg/kg.

Clinical Study in Patients with Relapse-Remitting Multiple Sclerosis

The procedure is carried out as described in Selmaj et al., Lancet Neurology, 2013, 12, 756-767, which is hereby incorporated by reference in its entirety. Compounds disclosed herein may be active (similar to siponimod) in reducing multiple sclerosis lesions in this assay, indicating that they are useful for the treatment of demyelinating diseases such as multiple sclerosis.

### GTPγS-Binding Assay

The procedure is carried out as described in Gergely et al., Brit. J. Pharmacol., 2012, 167, 1035-47, which is hereby incorporated by reference in its entirety. Compounds disclosed herein may demonstrate agonist activity at the S1P receptor in this assay; in certain embodiments, the compounds will be selective for S1P₁ and S1P₅ receptors.

### Agonist-Mediated Internalization of S1P₁ Receptor Assay

The procedure is carried out as described in Gergely et al., Brit. J. Pharmacol., 2012, 167, 1035-47, which is hereby incorporated by reference in its entirety. Compounds disclosed herein may induce S1P₁ receptor internalization in this assay.

### Electrophysiological Recordings of GIRK Channels in Human Atrial Myocytes

The procedure is carried out as described in Gergely et al., Brit. J. Pharmacol., 2012, 167, 1035-47, which is hereby incorporated by reference in its entirety. Compounds disclosed herein may have activity in this assay.

### EAE Induction Assay

The procedure is carried out as described in Gergely et al., Brit. J. Pharmacol., 2012, 167, 1035-47, which is hereby incorporated by reference in its entirety. Compounds disclosed herein may treat and/or prevent the development and/or symptoms of EAE in this assay, indicating that they are useful for the treatment of demyelinating diseases such as multiple sclerosis.

### Metabolic Profiling Assays

The procedure is carried out as described in WO 2014161606, which is hereby incorporated by reference in its entirety.

From the foregoing description, one skilled in the art can easily ascertain the essential characteristics of this disclosure, and without departing from the spirit and scope thereof, can make various changes and modifications of the disclosure to adapt it to various usages and conditions.

Reference throughout this specification to “one embodiment,” “certain embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

Although the disclosure herein has been described with reference to particular embodiments, it is to be understood that these embodiments are merely illustrative of the principles and applications of the present disclosure. It will be apparent to those skilled in the art that various modifications and variations can be made to the method and apparatus of the present disclosure without departing from the spirit and scope of the disclosure. Thus, it is intended that the present disclosure include modifications and variations that are within the scope of the appended claims and their equivalents.

What is claimed is:

1. A compound of Formula I 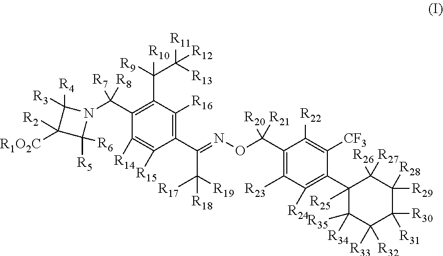
or a salt thereof, wherein:
R₁-R₃₅ are, independently, hydrogen or deuterium;
at least one of R₁-R₃₅ is deuterium; and
at least one of R₁-R₃₅ independently has deuterium enrichment of no less than about 1%.

2. The compound, or a salt thereof, of claim 1, wherein R₇ and R₈ are deuterium.

3. The compound, or a salt thereof, of claim 1 or 2, wherein R₉ and R₁₀ are deuterium.

4. The compound, or a salt thereof, of any one of the preceding claims, wherein R₇-R₁₀ are deuterium.

5. The compound, or a salt thereof, of any one of the preceding claims, wherein R₁₁-R₁₃ are deuterium.

6. The compound, or a salt thereof, of any one of the preceding claims, wherein R₇, R₈, and R₁₁-R₁₃ are deuterium.

7. The compound, or a salt thereof, of any one of the preceding claims, wherein R₉-R₁₃ are deuterium.

8. The compound, or a salt thereof, of any one of the preceding claims, wherein R₇-R₁₃ are deuterium.

9. The compound, or a salt thereof, of any one of the preceding claims, wherein R₁₇-R₁₉ are deuterium.

10. The compound, or a salt thereof, of any one of the preceding claims, wherein R₇, R₈, and R₁₇-R₁₉ are deuterium.

11. The compound, or a salt thereof, of any one of the preceding claims, wherein R₉, R₁₀, and R₁₇-R₁₉ are deuterium.

12. The compound, or a salt thereof, of any one of the preceding claims, wherein R₇-R₁₀ and R₁₇-R₁₉ are deuterium.

13. The compound, or a salt thereof, of any one of the preceding claims, wherein R₁₁-R₁₃ and R₁₇-R₁₉ are deuterium.

14. The compound, or a salt thereof, of any one of the preceding claims, wherein R₇, R₈, R₁₁-R₁₃, and R₁₇-R₁₉ are deuterium.

15. The compound, or a salt thereof, of any one of the preceding claims, wherein R₉-R₁₃ and R₁₇-R₁₉ are deuterium.

16. The compound, or a salt thereof, of any one of the preceding claims, wherein R₇-R₁₃ and R₁₇-R₁₉ are deuterium.

17. The compound, or a salt thereof, of any one of the preceding claims, wherein R₂₀ and R₂₁ are deuterium.

18. The compound, or a salt thereof, of any one of the preceding claims, wherein R₇, R₈, R₂₀, and R₂₁ are deuterium.

19. The compound, or a salt thereof, of any one of the preceding claims, wherein R₉, R₁₀, R₂₀, and R₂₁ are deuterium.

20. The compound, or a salt thereof, of any one of the preceding claims, wherein R₇-R₁₀, R₂₀, and R₂₁ are deuterium.

21. The compound, or a salt thereof, of any one of the preceding claims, wherein R₁₁-R₁₃, R₂₀, and R₂₁ are deuterium.

22. The compound, or a salt thereof, of any one of the preceding claims, wherein R₇, R₈, R₁₁-R₁₃, R₂₀, and R₂₁ are deuterium.

23. The compound, or a salt thereof, of any one of the preceding claims, wherein R₉-R₁₃, R₂₀, and R₂₁ are deuterium.

24. The compound, or a salt thereof, of any one of the preceding claims, wherein R₇-R₁₃, R₂₀, and R₂₁ are deuterium.

25. The compound, or a salt thereof, of any one of the preceding claims, wherein R₁₇-R₂₁ are deuterium.

26. The compound, or a salt thereof, of any one of the preceding claims, wherein R₇, R₈, and R₁₇-R₂₁ are deuterium.

27. The compound, or a salt thereof, of any one of the preceding claims, wherein R₉, R₁₀, and R₁₇-R₂₁ are deuterium.

28. The compound, or a salt thereof, of any one of the preceding claims, wherein R₇-R₁₀, and R₁₇-R₂₁ are deuterium.

29. The compound, or a salt thereof, of any one of the preceding claims, wherein R₁₁-R₁₃ and R₁₇-R₁₉ are deuterium.

30. The compound, or a salt thereof, of any one of the preceding claims, wherein R₇, R₈, R₁₁-R₁₃, and R₁₇-R₂₁ are deuterium.

31. The compound, or a salt thereof, of any one of the preceding claims, wherein R₉-R₁₃, and R₁₇-R₂₁ are deuterium.

32. The compound, or a salt thereof, of any one of the preceding claims, wherein R₇-R₁₃ and R₁₇-R₂₁ are deuterium.

33. The compound, or a salt thereof, of any one of the preceding claims, wherein R₇-R₁₃ and R₁₇-R₂₁ are deuterium.

34. The compound, or a salt thereof, of any one of the preceding claims, wherein R₁ is hydrogen.

35. The compound, or a salt thereof, of any one of the preceding claims, wherein R₁₄-R₁₆ are hydrogen.

36. The compound, or a salt thereof, of any one of the preceding claims, wherein R₂₂-R₂₄ are hydrogen.

37. The compound, or a salt thereof, of any one of the preceding claims, wherein R₁, R₁₄-R₁₆, and R₂₂-R₂₄ are hydrogen.

38. The compound, or a salt thereof, of any one the preceding claims, wherein at least one of R₁-R₃₅ independently has deuterium enrichment of no less than about 10%.

39. The compound, or a salt thereof, of any one of the preceding claims, wherein at least one of R₁-R₃₅ independently has deuterium enrichment of no less than about 50%.

40. The compound, or a salt thereof, of any one of the preceding claims, wherein at least one of R₁-R₃₅ independently has deuterium enrichment of no less than about 90%.

41. The compound, or a salt thereof, of any one of the preceding claims, wherein at least one of R₁-R₃₅ independently has deuterium enrichment of no less than about 98%.

42. The compound of claim 1 which is: 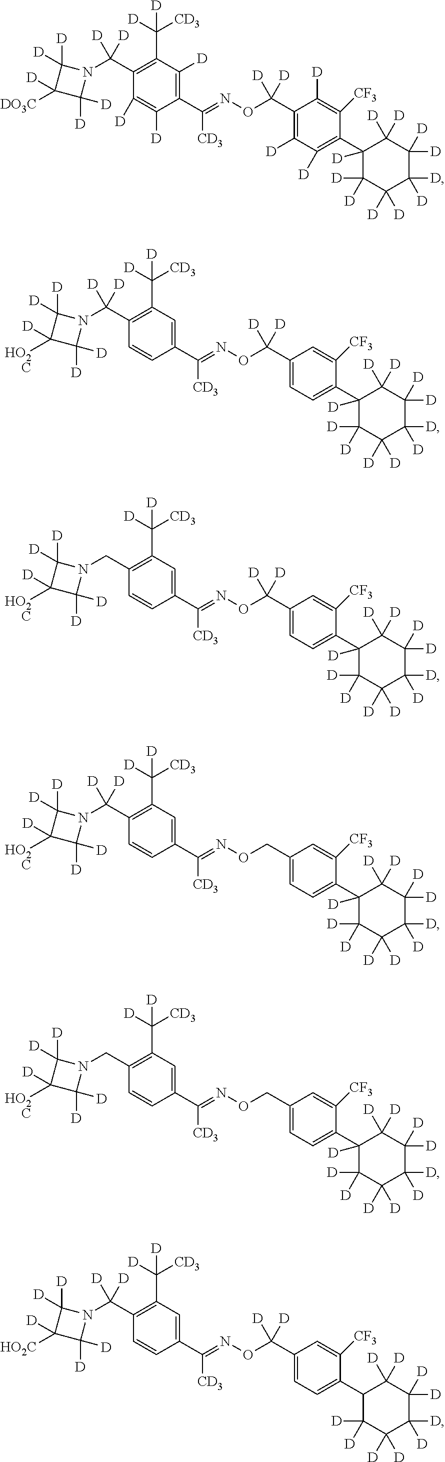 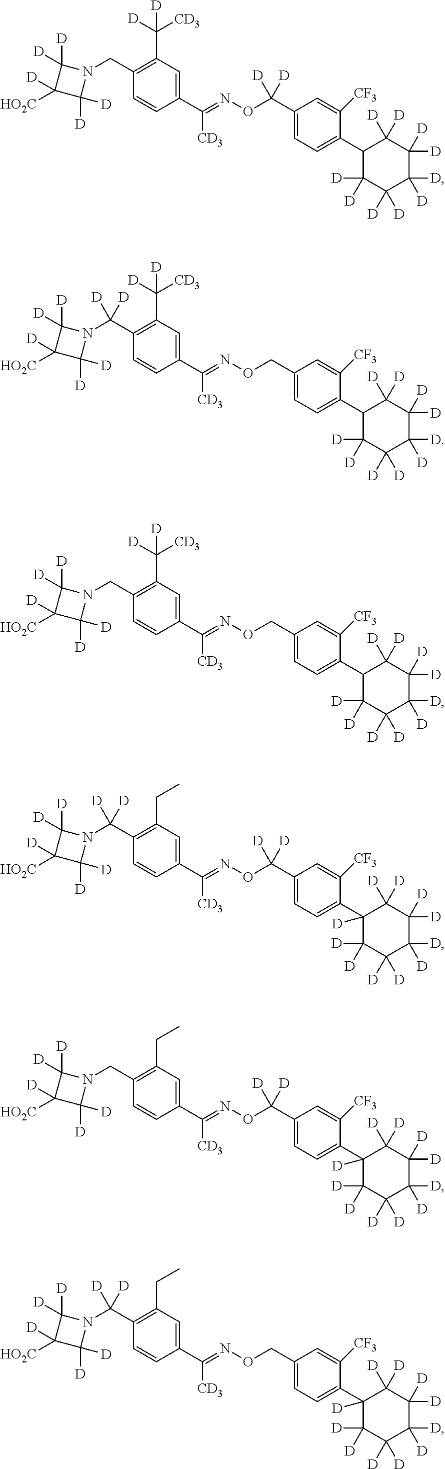 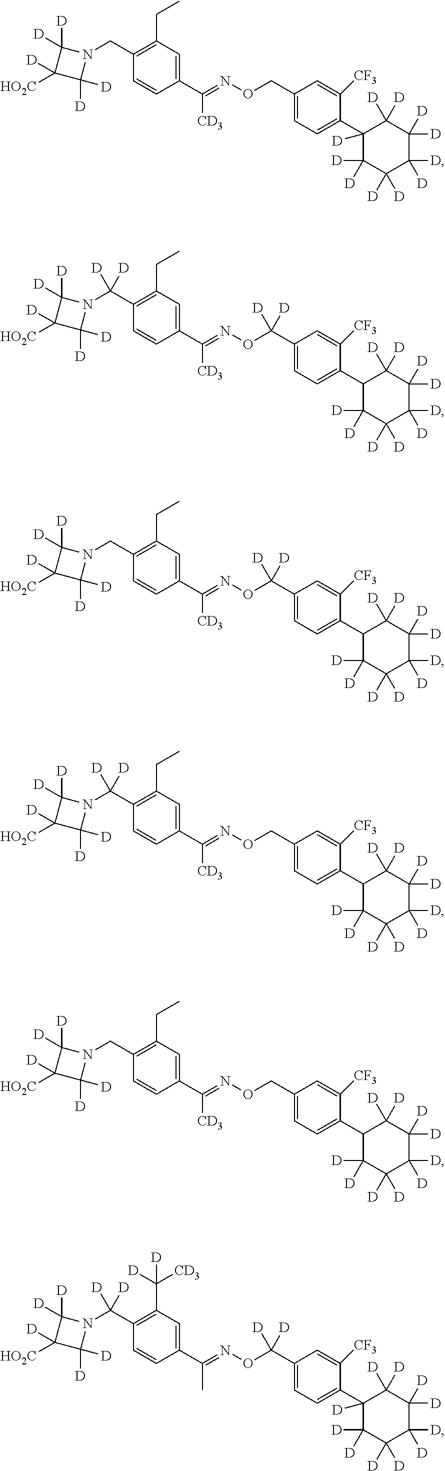 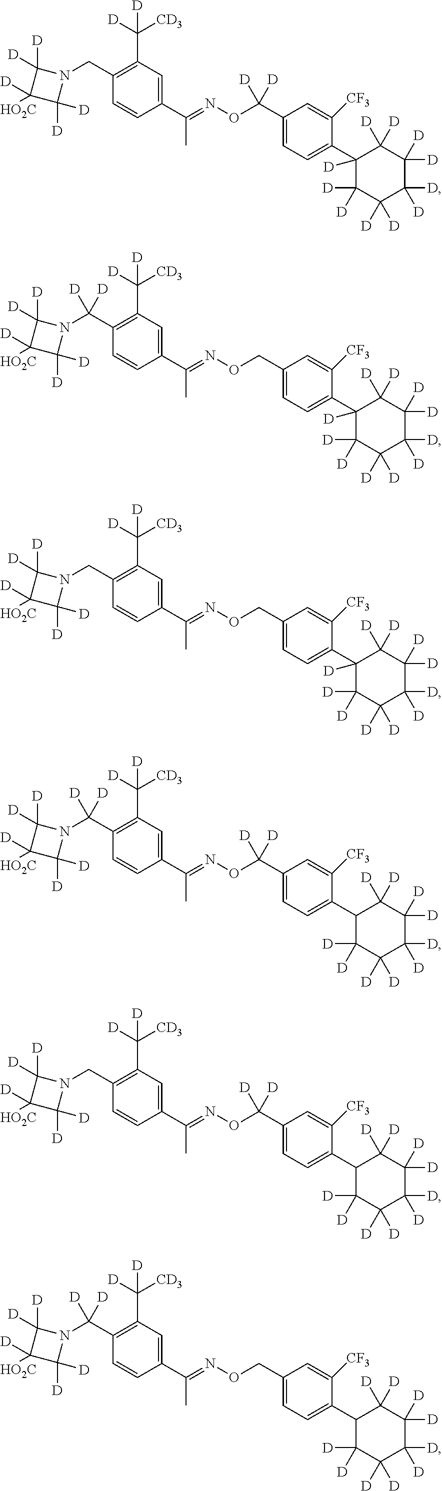 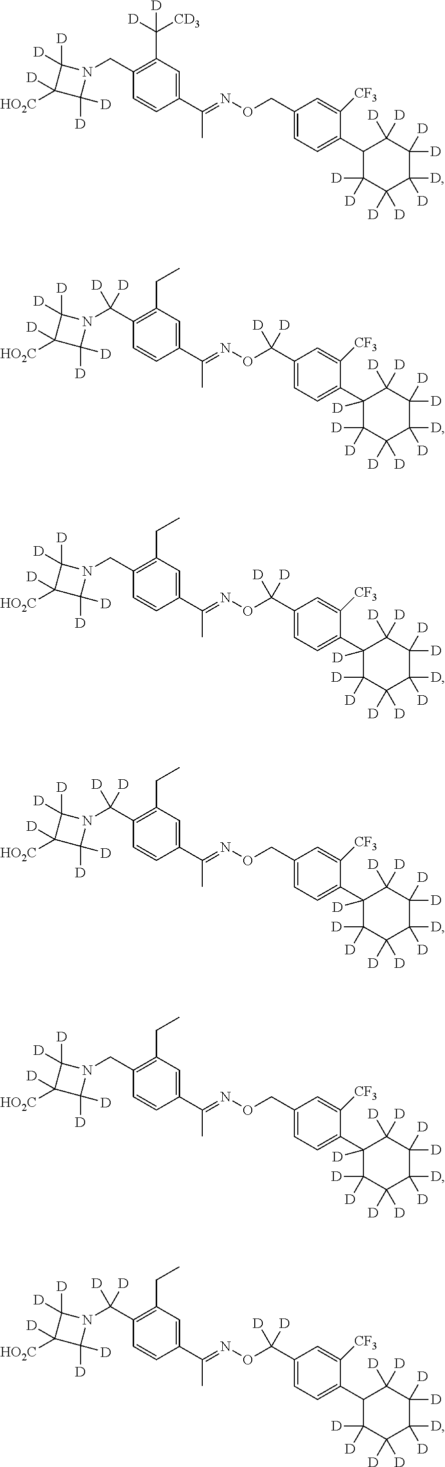 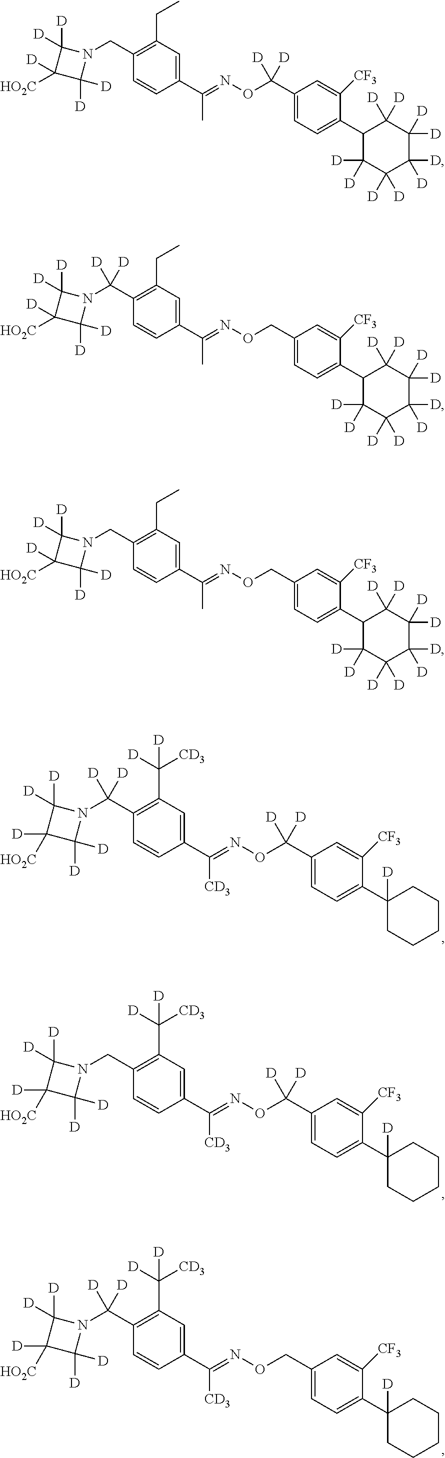 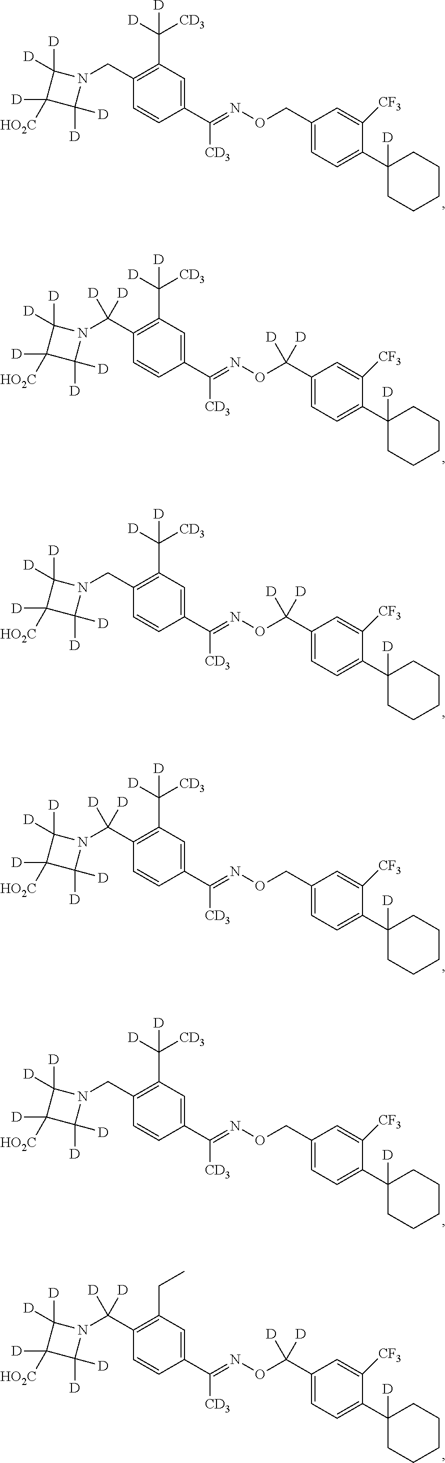 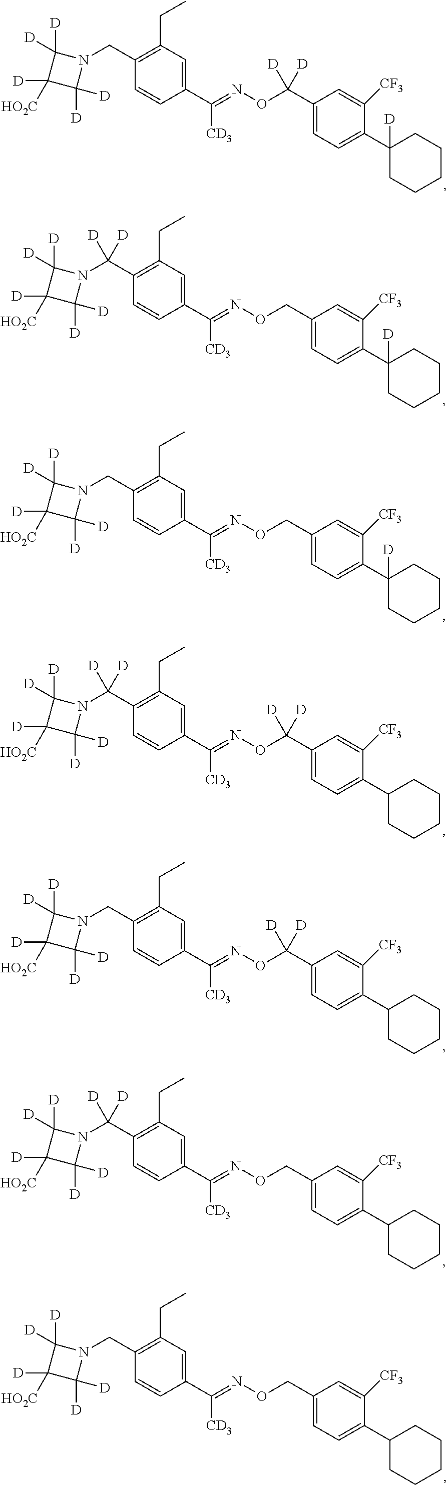 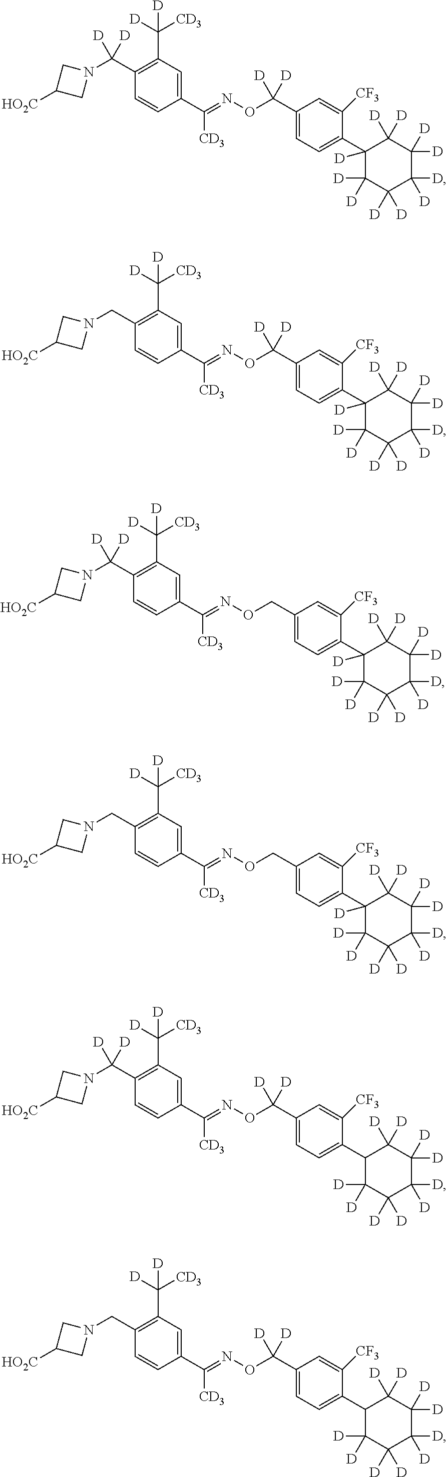 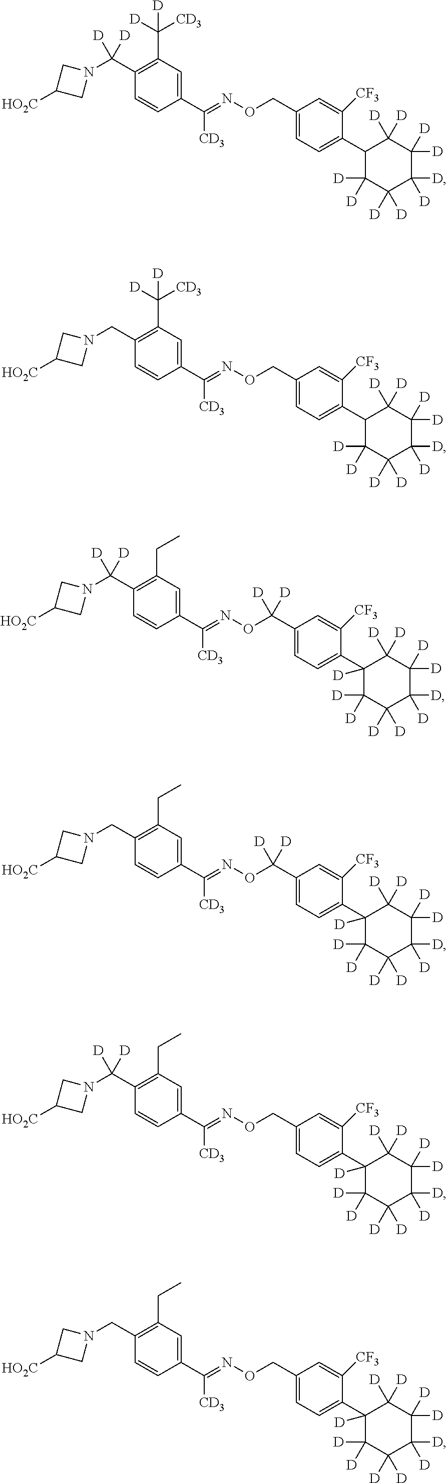 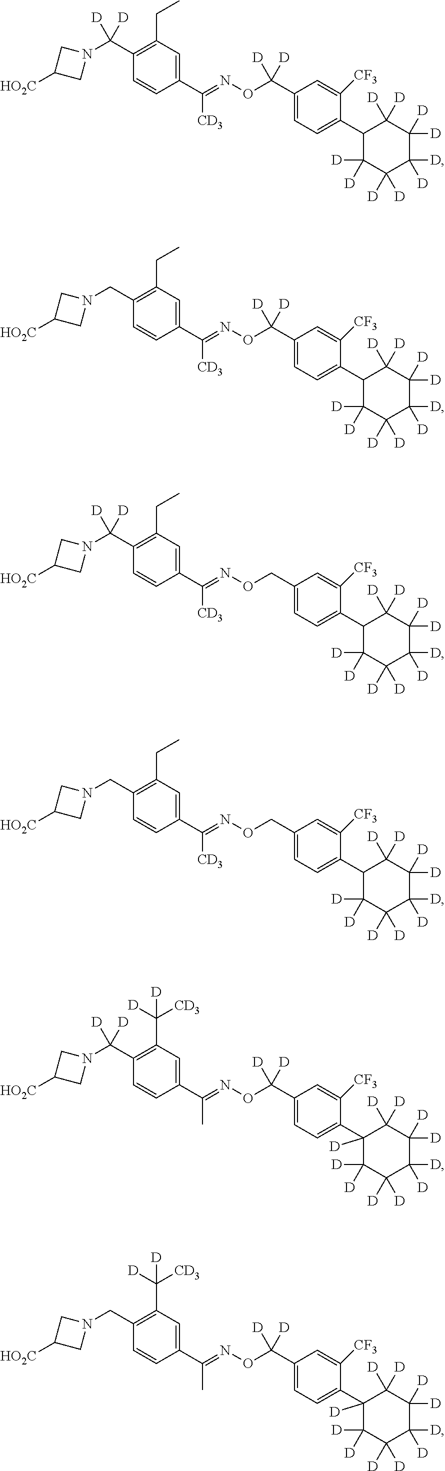 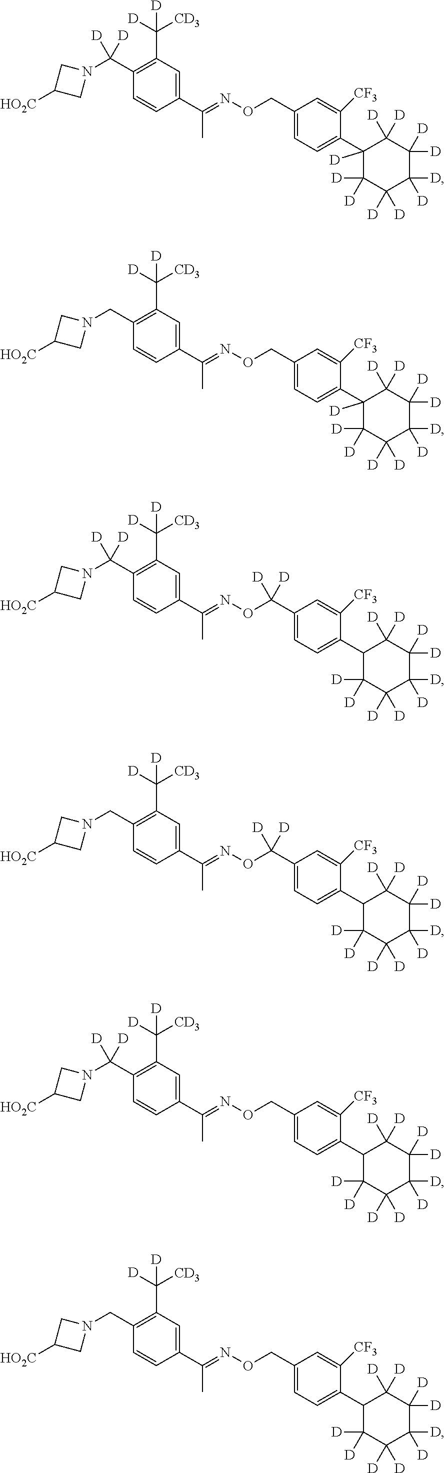 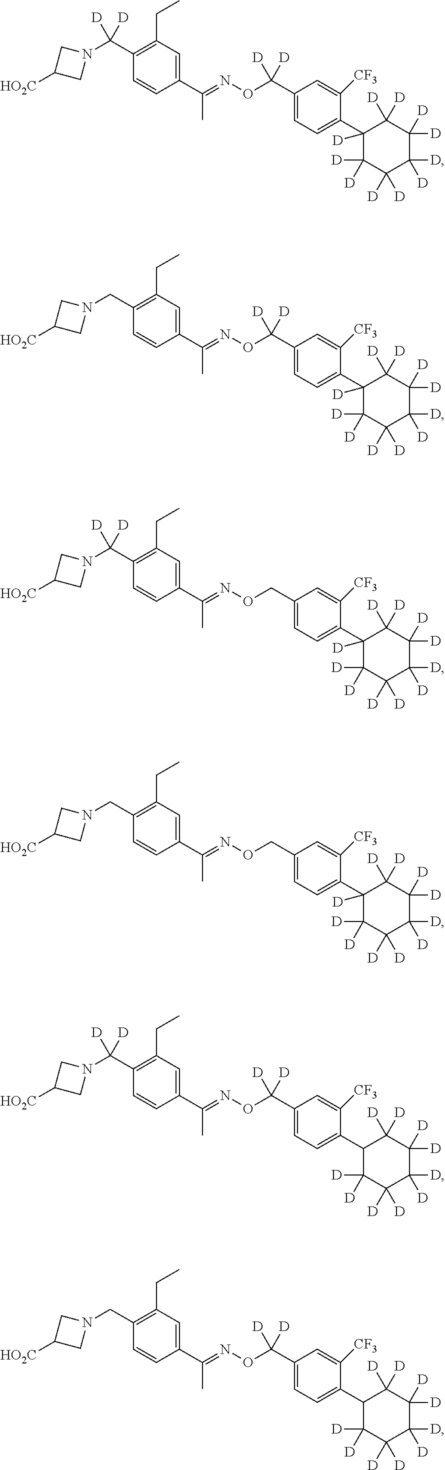 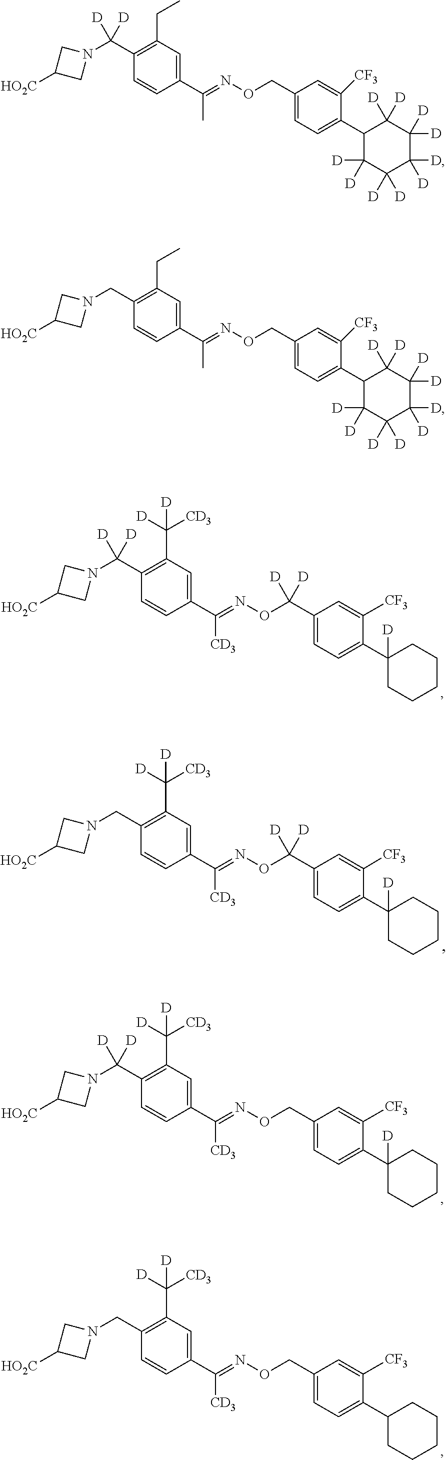 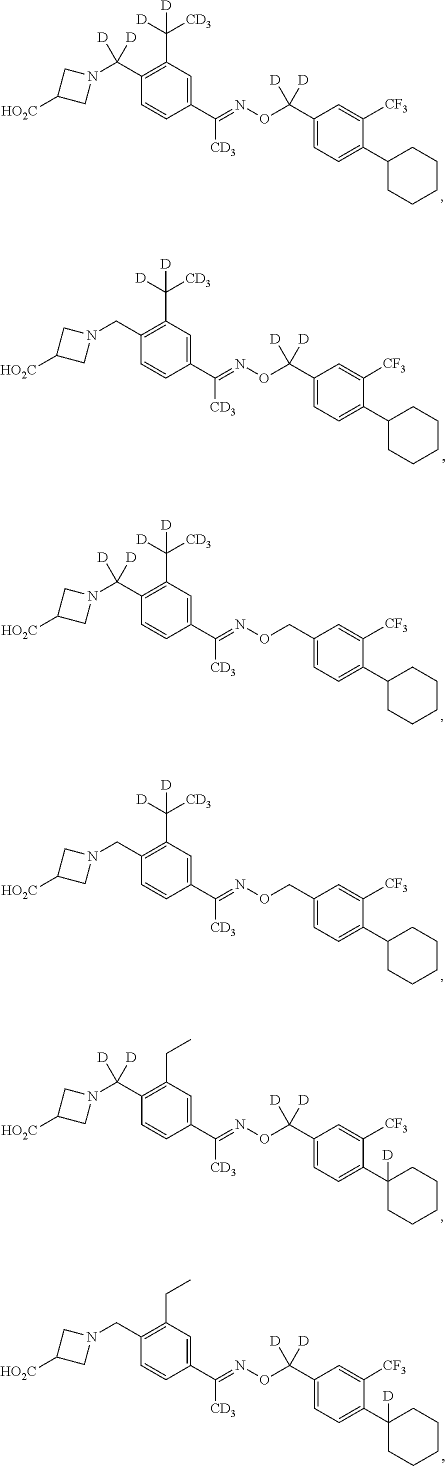 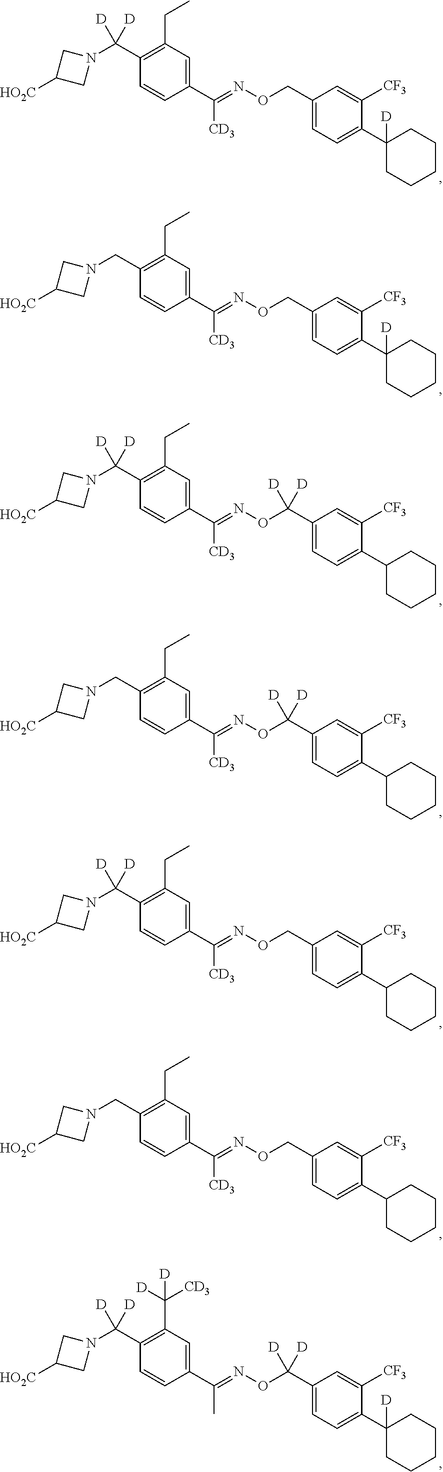 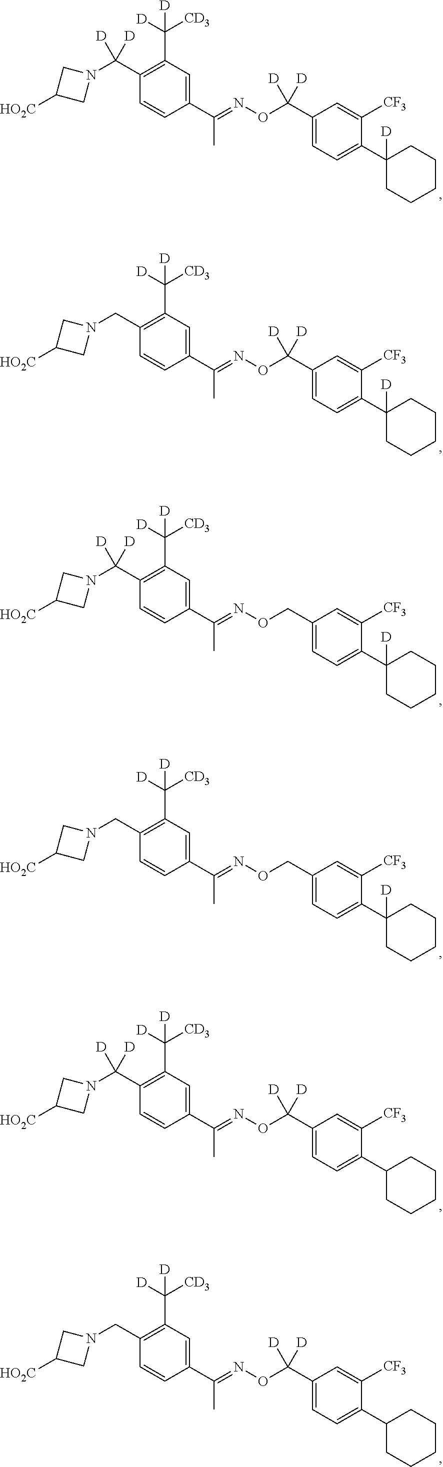 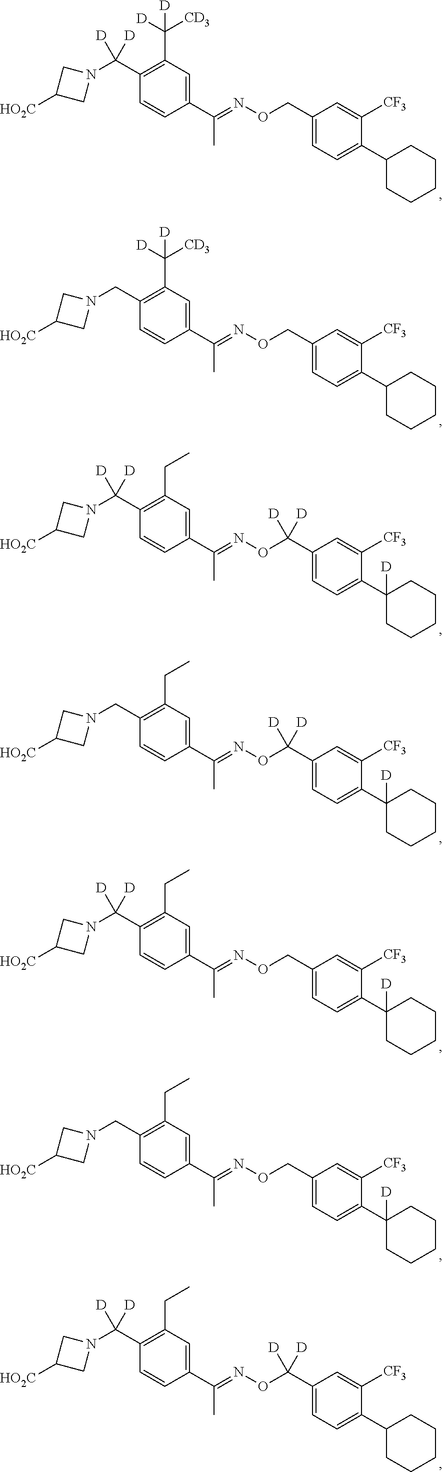 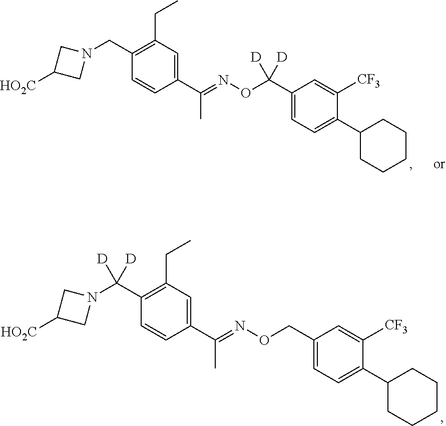 [SMILES: [2H]C([2H])(O/N=C(\C)c1ccc(CN2CC(C(=O)O)C2)c(CC)c1)c1ccc(C2CCCCC2)c(C(F)(F)F)c1 ; [2H]C([2H])(c1ccc(/C(C)=N/OCc2ccc(C3CCCCC3)c(C(F)(F)F)c2)cc1CC)N1CC(C(=O)O)C1]
or a salt thereof.

43. The compound of claim 1 which is: 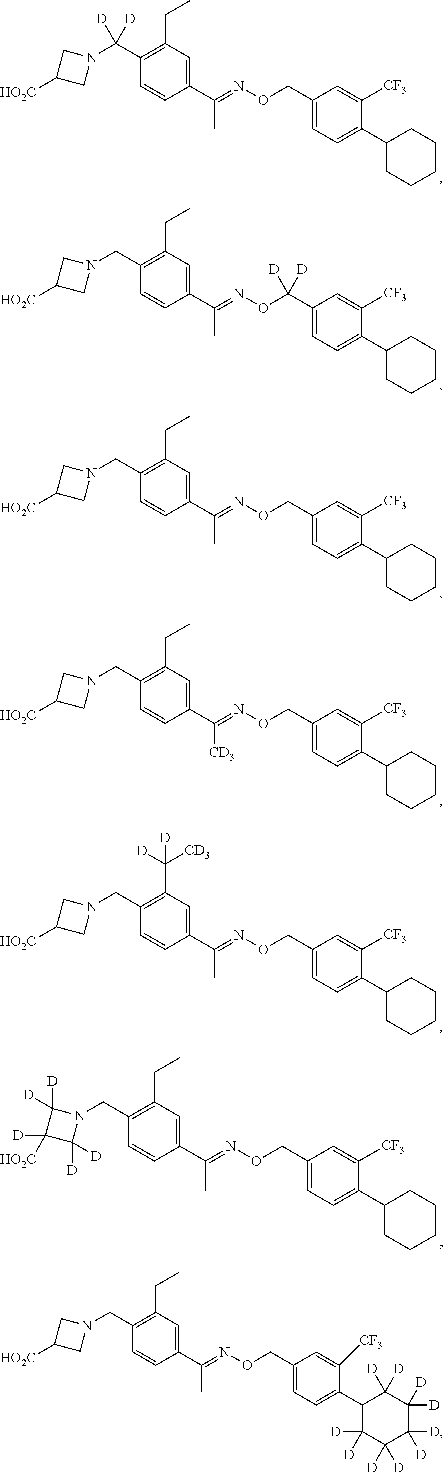 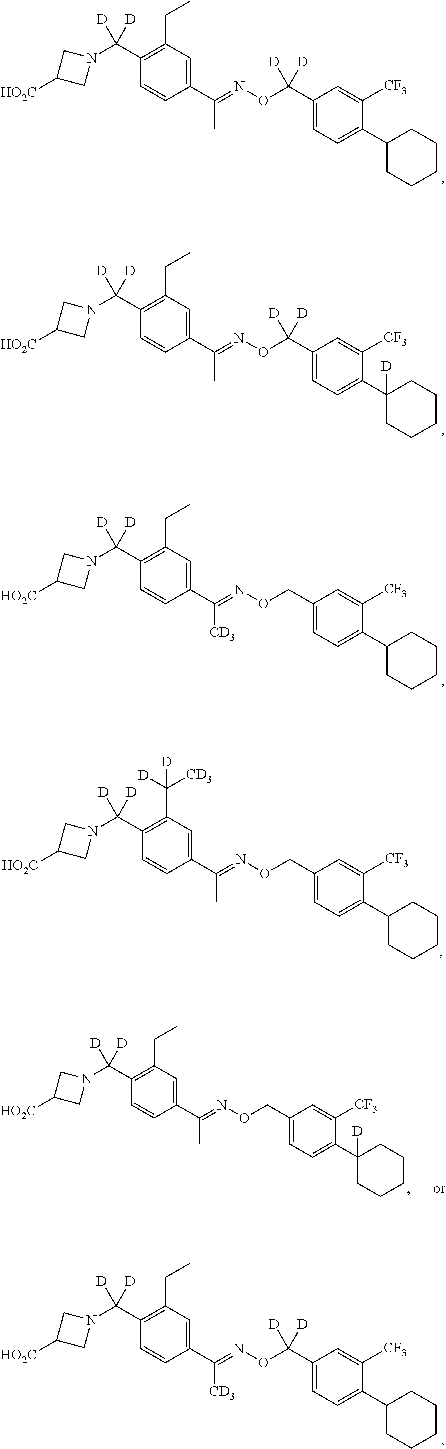
or a salt thereof.

44. The compound of claim 1 which is: 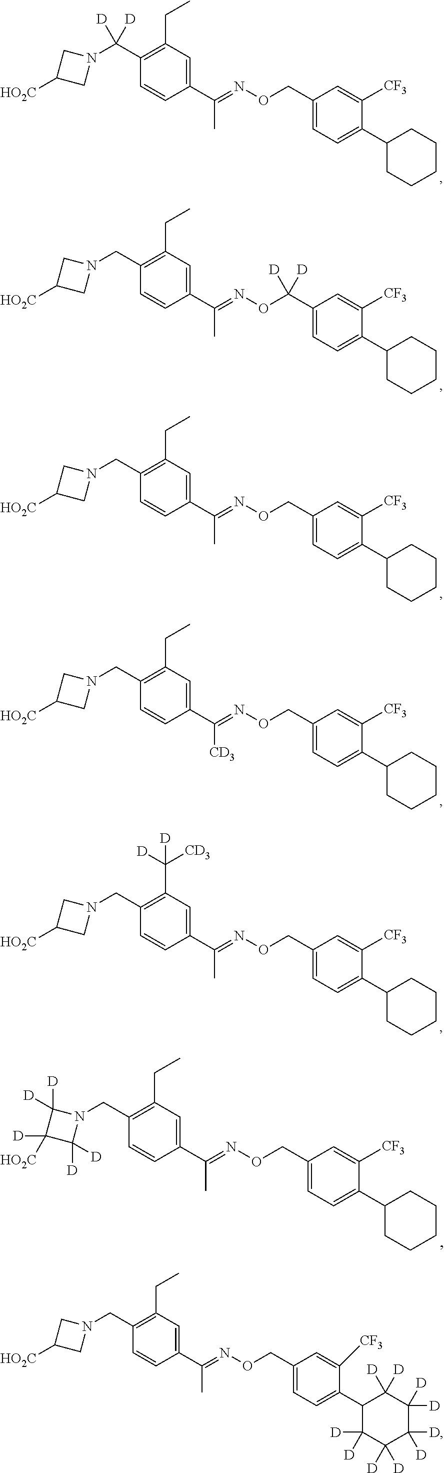 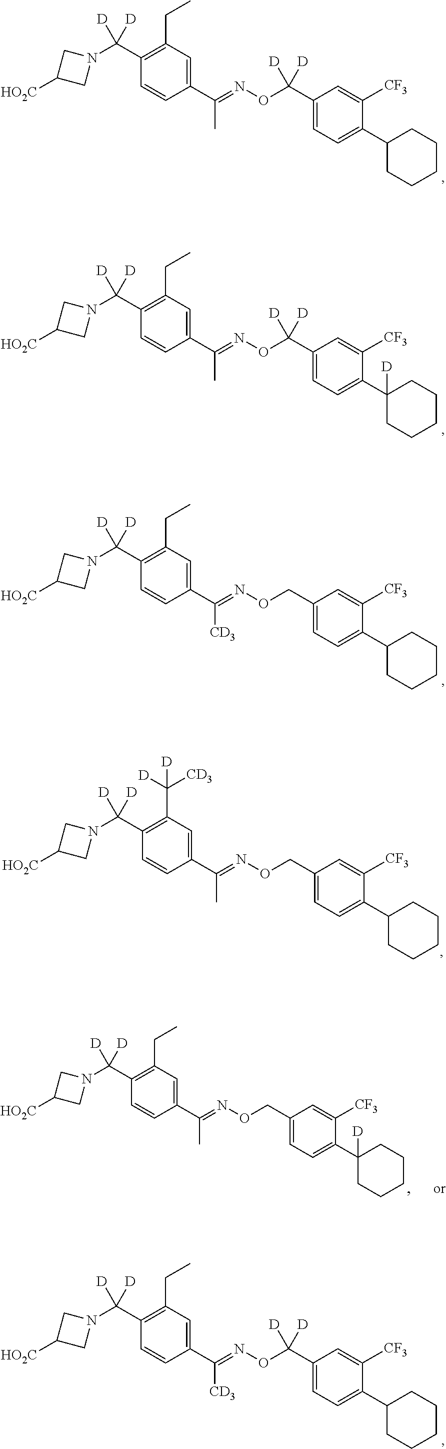
or a salt thereof.

45. The compound of claim 13 which is: 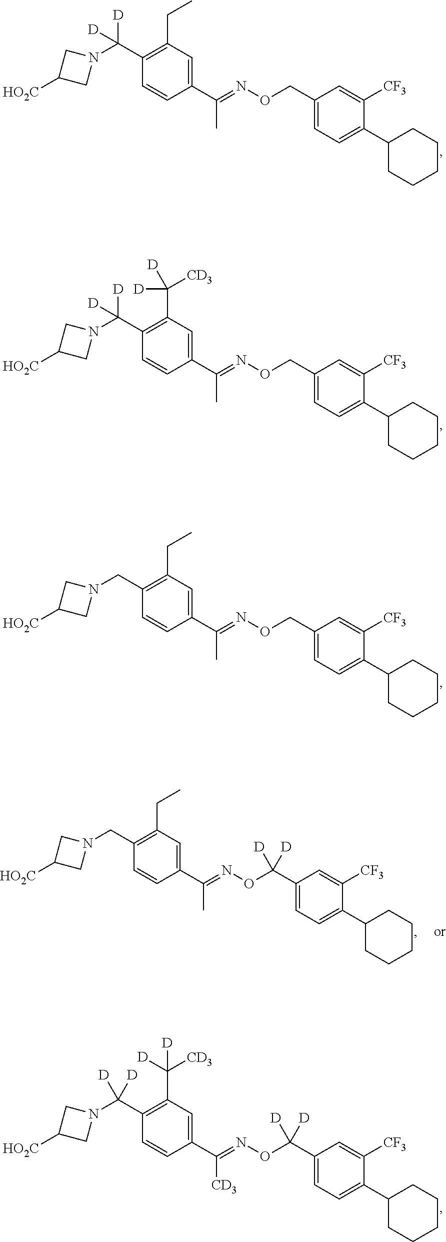 [SMILES: [2H]C([2H])(c1ccc(/C(C)=N/OCc2ccc(C3CCCCC3)c(C(F)(F)F)c2)cc1CC)N1CC(C(=O)O)C1 ; [2H]C([2H])(c1ccc(/C(C)=N/OCc2ccc(C3CCCCC3)c(C(F)(F)F)c2)cc1C([2H])([2H])C([2H])([2H])[2H])N1CC(C(=O)O)C1 ; CCc1cc(/C(C)=N/OCc2ccc(C3CCCCC3)c(C(F)(F)F)c2)ccc1CN1CC(C(=O)O)C1 ; [2H]C([2H])(O/N=C(\C)c1ccc(CN2CC(C(=O)O)C2)c(CC)c1)c1ccc(C2CCCCC2)c(C(F)(F)F)c1 ; [2H]C([2H])([2H])/C(=N\OC([2H])([2H])c1ccc(C2CCCCC2)c(C(F)(F)F)c1)c1ccc(C([2H])([2H])N2CC(C(=O)O)C2)c(C([2H])([2H])C([2H])([2H])[2H])c1]
or a salt thereof.

46. The compound, or a salt thereof, of any one of claims 42-45, wherein each position represented as D has deuterium enrichment of no less than about 1%.

47. The compound, or a salt thereof, of any one of claims, 42-45 wherein each position represented as D has deuterium enrichment of no less than about 10%.

48. The compound, or a salt thereof, of any one of claims, 42-45 wherein each position represented as D has deuterium enrichment of no less than about 50%.

49. The compound, or a salt thereof, of any one of claims, 42-45 wherein each position represented as D has deuterium enrichment of no less than about 90%.

50. The compound, or a salt thereof, of any one of claims, 42-45 wherein each position represented as D has deuterium enrichment of no less than about 98%.

51. A pharmaceutical composition comprising a compound, or a salt thereof, of any one of the preceding claims and a pharmaceutically acceptable carrier.

52. A method of treating a sphingosine 1-phosphate receptor-mediated disorder comprising administering a therapeutically effective amount of a compound, or a salt thereof, of any one of claims 1 to 50 to a patient in need thereof.

53. The method of claim 52 wherein the disorder is multiple sclerosis, secondary progressive multiple sclerosis, acute or chronic rejection of cell, tissue or organ allo- or xenografts, delayed graft function, graft versus host disease, an autoimmune disease, rheumatoid arthritis, systemic lupus erythematosus, Hashimoto's thyroiditis, myasthenia gravis, diabetes type I, diabetes type II, disorders associated with type I or II diabetes, vasculitis, pernicious anemia, Sjogren's syndrome, uveitis, psoriasis, Graves ophthalmopathy, alopecia areata, an allergic disease, allergic asthma, atopic dermatitis, allergic rhinitis, conjunctivitis, allergic contact dermatitis, an inflammatory disease, inflammatory bowel disease, Crohn's disease, ulcerative colitis, intrinsic asthma, inflammatory lung injury, inflammatory liver injury, inflammatory glomerular injury, atherosclerosis, osteoarthritis, irritant contact dermatitis, eczematous dermatitis, seborrheic dermatitis, a cutaneous manifestation of an immunologically-mediated disorder, inflammatory eye disease, keratoconjunctivitis, inflammatory myopathy, myocarditis, hepatitis, ischemia/reperfusion injury, myocardial infarction, stroke, gut ischemia, renal failure, hemorrhage shock, traumatic shock, T cell lymphoma, T cell leukemia, an infectious disease, toxic shock, septic shock, adult respiratory distress syndrome, a viral infection, AIDS, viral hepatitis, chronic bacterial infection, a muscle disease, polymyositis, senile dementia, pancreatic islet transplant, stem cell transplant, bone marrow transplant, corneal tissue transplant, neuronal tissue transplant, heart transplant, lung transplant, combined heart-lung transplant, kidney transplant, liver transplant, bowel transplant, pancreas transplant, trachea transplant, esophagus transplant, cancer chemotherapy, cancer chemotherapy of a solid tumor, breast cancer, peripheral neuropathy, an acute demyelinating neuropathy, a chronic demyelinating neuropathy, Guillain-Barre syndrome, chronic inflammatory demyelinating polyradiculoneuropathy, multifocal motor neuropathy with conduction block, paraproteinaemic demyelinating peripheral neuropathy, acute inflammatory demyelinating polyneuropathy, polymyositis, dermatomyositis, a nerve-muscle disease, muscular dystrophy, or inclusion body myositis.

54. The method of claim 53, wherein the disorder is multiple sclerosis.

55. The method of claim 52, further comprising administering an additional therapeutic agent.

56. The method of claim 55, wherein the additional therapeutic agent is a glucocorticoid or an immunosuppressant.

57. The method of claim 56, wherein the glucocorticoid is beclometasone, budesonide, flunisolide, betamethasone, fluticasone, triamcinolone, mometasone, ciclesonide, hydrocortisone, cortisone acetate, prednisone, prednisolone, methylprednisolone, or dexamethasone.

58. The method of claim 56, wherein the immunosuppressant is CP-690550, fingolimod, cyclosporine A, azathioprine, dexamethasone, tacrolimus, sirolimus, pimecrolimus, mycophenolate salts, everolimus, basiliximab, daclizumab, anti-thymocyte globulin, anti-lymphocyte globulin, or CTLA4IgG.

59. The method of claim 52, further resulting in at least one effect which is:
a. decreased inter-individual variation in plasma levels of the compound or a metabolite thereof as compared to the non-isotopically enriched compound;
b. increased average plasma levels of the compound per dosage unit thereof as compared to the non-isotopically enriched compound;
c. decreased average plasma levels of at least one metabolite of the compound per dosage unit thereof as compared to the non-isotopically enriched compound;
d. increased average plasma levels of at least one metabolite of the compound per dosage unit thereof as compared to the non-isotopically enriched compound; or
e. an improved clinical effect during the treatment in the subject per dosage unit thereof as compared to the non-isotopically enriched compound.

60. The method of claim 52, further resulting in at least two effects which are:
a. decreased inter-individual variation in plasma levels of the compound or a metabolite thereof as compared to the non-isotopically enriched compound;
b. increased average plasma levels of the compound per dosage unit thereof as compared to the non-isotopically enriched compound;
c. decreased average plasma levels of at least one metabolite of the compound per dosage unit thereof as compared to the non-isotopically enriched compound;
d. increased average plasma levels of at least one metabolite of the compound per dosage unit thereof as compared to the non-isotopically enriched compound; or
e. an improved clinical effect during the treatment in the subject per dosage unit thereof as compared to the non-isotopically enriched compound.

61. The method of claim 52, wherein the method effects a decreased metabolism of the compound per dosage unit thereof by at least one polymorphically-expressed cytochrome P₄₅₀ isoform in the subject, as compared to the corresponding non-isotopically enriched compound.

62. The method of claim 61, wherein the cytochrome P₄₅₀ isoform is CYP2C8, CYP2C9, CYP2C19, or CYP2D6.

63. The method of claim 52, wherein the compound is characterized by decreased inhibition of at least one cytochrome P₄₅₀ or monoamine oxidase isoform in the subject per dosage unit thereof as compared to the non-isotopically enriched compound.

64. The method of claim 63, wherein the cytochrome P₄₅₀ or monoamine oxidase isoform is CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, CYP51, MAO_{A}, or MAO_{B}.

65. The method of claim 52, wherein the method reduces a deleterious change in a diagnostic hepatobiliary function endpoint, as compared to the corresponding non-isotopically enriched compound.

66. The method of claim 65, wherein the diagnostic hepatobiliary function endpoint is alanine aminotransferase (“ALT”), serum glutamic-pyruvic transaminase (“SGPT”), aspartate aminotransferase (“AST,” “SGOT”), ALT/AST ratios, serum aldolase, alkaline phosphatase (“ALP”), ammonia levels, bilirubin, gamma-glutamyl transpeptidase (“GGTP,” “γ-GTP,” “GGT”), leucine aminopeptidase (“LAP”), liver biopsy, liver ultrasonography, liver nuclear scan, 5′-nucleotidase, or blood protein.

67. A compound, or a salt thereof, of any one of claims 1-50 for use as a medicament.

68. A compound, or a salt thereof, of any one of claims 1-50 for use in the manufacture of a medicament for preventing or treating a sphingosine 1-phosphate receptor-mediated disorder.
